(19) United States
(12) Patent Application Publication
(10) Pub. No.: US 2025/0127774 A1
(43) Pub. Date: Apr. 24, 2025

(54) COMPOUNDS FOR LABELLING NUCLEIC ACID AND USES THEREOF

(71) Applicants: THE ROYAL INSTITUTION FOR THE ADVANCEMENT OF LEARNING/MCGILL UNIVERSITY, Montréal (CA); TECHNICAL UNIVERSITY OF MUNICH, Munich (DE)

(72) Inventors: Morten O. LOEHR, Krefeld (DE); Nathan William LUEDTKE, Mont Royal (CA); Kaifeng ZHAO, Shenyang (CN); Daria STOMAKHIINE, Saint-Constant (CA); Yizhu LI, Munich (DE)

(21) Appl. No.: 18/836,290

(22) PCT Filed: Feb. 6, 2023
(86) PCT No.: PCT/CA2023/050149

§ 371 Date: Aug. 6, 2024

Related U.S. Application Data
(60) Provisional application No. 63/307,778, filed on Feb. 8, 2022.

(51) Int. Cl. A61K 31/473; A61P 35/00; C07D 401/04; C07D 401/06; C07D 401/12; C07D 401/14; C07D 471/16

(52) U.S. Cl. CPC A61K 31/473; A61P 35/00; C07D 401/04; C07D 401/06; C07D 401/12; C07D 401/14; C07D 471/16

(57) ABSTRACT

Theranostic compounds are provided having formula:

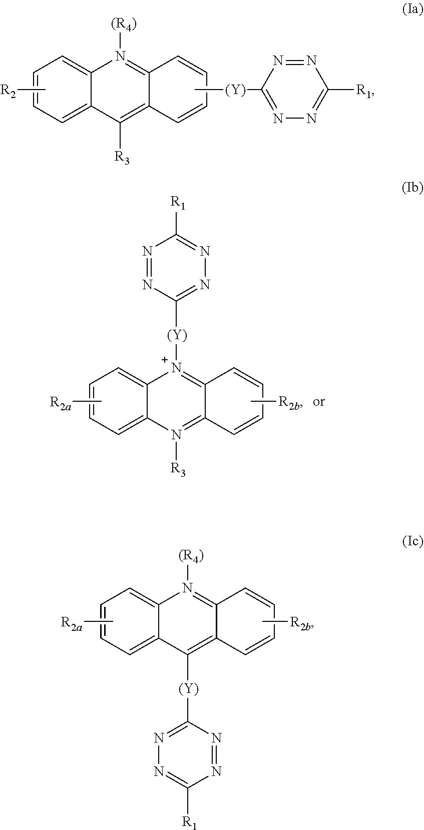

or a salt thereof.

### FIELD

The present disclosure generally relates to theranostic agents and compounds, namely compounds for labelling DNA and therapeutic uses thereof.

### BACKGROUND

Metabolic labeling of nucleic acids is a powerful technique to decipher the timing and location of DNA synthesis in vivo. The technique has been a cornerstone for cell cycle analysis, but also been extended to be used for viral detection and the elucidation of drug resistance mechanisms. In general, a nucleoside of interest is functionalized with a synthetic handle, which is small enough to conserve the original biological function, but also allows for chemoselective detection and visualization of the nucleoside post incorporation.

Since the traditional immunostaining approach of 5-bromo-2′-deoxyuridine (BrdU) suffered from poor signal intensity and tissue permeability of the antibodies, biorthogonal chemical approaches were developed. Notably, these include, labeling of azide-or ethynyl-modified nucleosides by means of copper-catalyzed (CuAAC) or strain-promoted azide-alkyne cycloaddition (SPAAC), or alternatively, reaction of vinyl-nucleosides with tetrazines by an inverse electron-demand Diels-Alder reaction (iEDDA).

However, several nucleoside analogs, such as the widely used 5-ethynyl-2′-deoxyuridine (EdU) can heavily affect nucleic acid metabolism, the cell cycle, or worst be toxic to cells and organisms. In addition, most approaches for metabolic labeling of nucleic acids still require fixation of biological samples or denaturation of DNA, which prevents numerous long-term, in vivo applications. Accordingly, improved compounds and methods for metabolic labeling of nucleic acid are needed.

### SUMMARY

In one aspect, there is provided a compound having a formula:

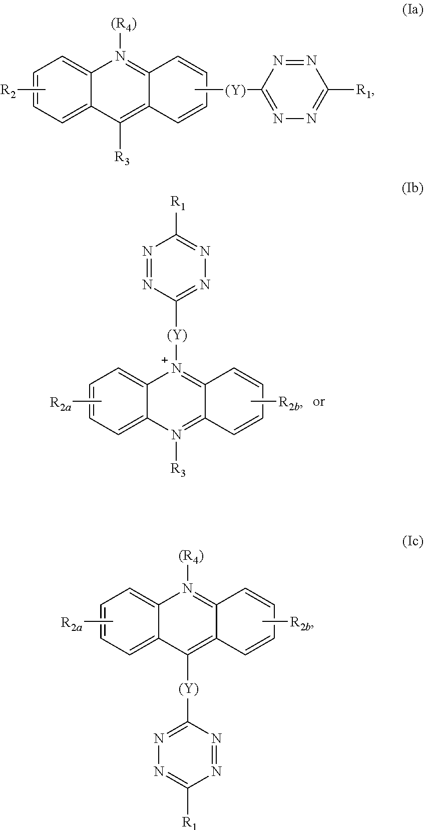

or a salt thereof; wherein: R₁ is H, haloalkyl, alkyl, alkenyl, alkynyl, cycloalkyl, aryl, heteroaryl, heterocyclyl, or an optionally substituted group thereof; R₂, R₂ₐ, R_{2b}, and R₃ are independently H, hydroxy, halogen, haloalkyl, cyano, amino, thiol, alkyl, alkenyl, alkynyl, cycloalkyl, aryl, heteroaryl, heterocyclyl, alkoxy, sulfenyl, acyl, sulfinyl, sulfonyl, O-carboxy, ester, thiocarbonyl, or an optionally substituted group thereof; R₄ is an optional group and is H, haloalkyl, alkyl, alkenyl, alkynyl, cycloalkyl, aryl, heteroaryl, heterocyclyl, or an optionally substituted group thereof; and Y is an optional linker.

In another aspect, there is provided a method of detecting nucleic acids containing a vinyl-nucleoside, the method comprising: a) contacting the cell or tissue with the compound or salt as described herein; and exposing the cell or tissue of a) to light.

In another aspect, there is provided a kit for detection of nucleic acid in a cell or tissue, the kit comprising one or more vinyl-nucleosides; and the compound or salt as described herein.

In another aspect, there is provided a method of treating cancer in a subject that has been administered one or more vinyl-nucleosides, the method comprising administering the compound or salt as described herein to the subject.

In another aspect, there is provided a pharmaceutical composition comprising the compound or salt as described herein and a acceptable pharmaceutical carrier.

In another aspect, there is provided use of the compound or salt as described herein in the manufacture of a medicament for the treatment of cancer. In yet another aspect, there is provided use of the compound or salt as described herein, or the pharmaceutical composition described herein, for the treatment of cancer.

In another aspect, there is provided use of the compound or salt as described herein, or the pharmaceutical composition described herein, in combination with one or more vinyl-nucleosides in the treatment of cancer.

### DESCRIPTION OF THE FIGURES

Embodiments of devices, apparatus, methods, and kits are described throughout reference to the drawings.

FIG. 1 shows a scheme where vinyl-modified, cellular DNA is produced by the addition of VdU to living cells or animals. PINK is then added to the cells and it “scans” the DNA for alkene groups via reversible interaction.

FIG. 2 shows a model of PINK pi-stacked on a VdU-dA base pair.

FIG. 3 shows photophysical and DNA binding properties of PINK. a) Absorbance changes of a 10 μM solution of PINK with increasing amounts of calf thymus DNA (0-160 UM base pairs; light to dark orange) in 50 mM NaOAc buffer pH 5.2 (0.2% DMSO). b) Viscosity changes of a 300 μM solution of calf thymus DNA with increasing amounts of PINK (0-120 μM) in 50 mM NaOAc buffer pH 5.2 (2.4% DMSO). c) Increasing fluorescence of a 25 μM solution of PINK containing 5 mM VdU in MeCN 9:1 DMSO over 46 h (excitation: 480 nm) d) Absorbance and fluorescence spectra (excitation: 500 nm) of a 50 μM solution of PINK-VdU-ox (6) in various solvents and phosphate-buffer saline (PBS).

FIG. 4 shows A) the absorbance changes at 575 nm of a solution of 10 μM PINK with increasing amounts of CT-DNA (0-160 μM base pairs) in 50 mM NaOAc buffer pH 5.2 (0.2% DMSO) yields an effective concentration (EC₅₀)=50±5.1 μM base pairs needed to bind 50% of PINK. B) Fluorescence changes (excitation: 500 nm, emission: 590 nm; 515 nm cutoff filter) of 20 μM PINK in the presence of 1 mM b.p. CT-DNA (50 eq) in PBS pH 7.4 (10% DMSO) followed by addition of 5-norbornene-2-methanol (2 mM, 100 eq).

FIG. 5 shows kinetic analysis of PINK (100 μM) reacting with VdU (0-5 mM, 0-50 eq). A) Fluorescence increase versus time upon mixing PINK with VdU. Control samples (grey) did not contain VdU. B) Normalized increase of fluorescence. C) Plot of the observed rates k′ versus PINK concentrations. The slope yields the rate constant k.

FIG. 6 shows photophysical characterization of PINK. 20 μM PINK in aq. NaOAc buffer pH 5.2 (red) or MeCN (blue); 1% DMSO. A) Full Absorbance spectrum. B) Absorbance (solid) and emission (dashed; excitation 488 nm, 495 nm cutoff filter).

FIG. 7 shows photophysical properties of PINK-VdU-ox (50 μM, 0.5% DMSO) in various solvents. A) Absorbance spectrum. B) Emission spectrum (excitation at 500 nm; 515 nm cutoff filter). [a] 40% PEG200 in PBS pH 7.4.

FIG. 8 shows kinetic measurements of PINK with vinyl-modified ODN. A) Relative fluorescence units (RFU) of PINK upon reaction with vinyl-modified ODN versus time. Control samples (dashed) did not contain DNA. B) Normalized increase of fluorescence. C) Plot of the observed rates k′ against PINK concentration. The slope yields the rate constant k.

FIG. 9 shows DNA metabolic labeling and imaging of living Hela cells. Control (ctrl) samples were treated with PINK but not VdU. Scale bars represent 40 μm.

FIG. 10 shows PINK staining kinetics. Scale bars represent 40 μm.

FIG. 11 shows live-cell metabolic labeling of VdU in U2OS cells.

FIG. 12 shows long term live cell labeling of Hela cells. Scalebars represent 40 μM.

FIG. 13 shows metabolic RNA labeling. Scalebars represent 100 μm (a) or 40 μm (b) respectively.

FIG. 14 shows a) Pulse-Chase Labeling with PINK and 5-ethynyl-2′-deoxyuridine in Hela cells. “No VdU” cells were incubated with 1 μM PINK but not VdU. Scalebars represent 100 μm. b) Live cell time lapse imaging of a full mitotic cyclic after labeling with 100 μM VdU. Scalebars represent 20 μm.

FIG. 15 shows absorbance (dashed) and fluorescence emission (solid) spectra of AO-495-tet-py before (black) and after (red) reaction with 5-norbornene-2 methanol (in PBS).

FIG. 16 shows toxicity evaluation of PINK (0-12.5 μM; 0.1% DMSO) in HeLa and U2OS cells after 24 and 72 h. Cellular respiratory activity was evaluated by measuring the fluorescence emission after addition of resazurin (80 M).

FIG. 17 shows combined Toxicity of VdU (0-100 M) and PINK (0, 5, 10 μM). Experiment performed in triplicates; error bars depict the standard deviation.

FIG. 18 shows combined Toxicity of VdU (0-10, 20 μM) and PINK (0-100 μM). Experiment performed in triplicates; error bars depict the standard deviation.

FIG. 19 shows cell viability based on resazurin reduction by the metabolic activities of MV4-11 and Jurkat cells after 24 h and 72h incubation with variable concentration of VdU, VdA and PINK. Values were normalized relative to untreated (DMSO+media) cells. Three technical replicates were performed, and each value is given as the mean±SD.

FIG. 20 shows cell viability based on resazurin reduction by the metabolic activities of MV4-11, Jurkat cells after 48h incubation with variable concentration of VdU/VdA alone and in combinations with 1 μM of PINK (added 24h post nucleoside addition). Values were normalized relative to untreated (DMSO+media) cells. Three technical replicates were performed, and each value is given as the mean±SD.

FIG. 21 shows cell viability based on resazurin reduction by the metabolic activities of MV4-11, Jurkat cells after 48h incubation with variable concentration of VdU/VdA alone and in combinations with 1 μM of PINK (added on the same day as the nucleosides). Values were normalized relative to untreated (DMSO+media) cells. Three technical replicates were performed, and each value is given as the mean±SD.

FIGS. 22A-F show relative cell viability and cell concentration of Jurkat, MV4-11 and MOLM-13 treated with VdU, VdA and PINK alone and in combinations. Two technical replicates were performed, and each value is given as the mean±SD. A) Relative viability plot of Jurkat. B) Concentration plot of Jurkat. C) Relative viability plot of MV4-11. D) Concentration plot of MV4-11. E) Relative viability plot of MOLM-13. F) Concentration plot of MOLM-13.

FIGS. 23A-C shows Fa-CI plot of MV4-11, Jurkat and MOLM-13 cells treated at different concentrations of VdU and PINK alone and in combination with a constant 5:1 VdU to PINK ratio. Fa=Fraction affected and CI=Combination Index. A) Fa-CI plot of MV4-11 with an average CI=0.157 for 25-35% of cells killed. B) Fa-CI plot for Jurkat with an average CI=0.486 for 50% cells killed. C) Fa-Cl plot of MOLM-13 with an average CI=0.684 for 0.684 for 50% cells killed.

FIG. 24 shows 5-VdU and PINK combinatory impact on cellular survival and DNA damage induction. (A) Treatment schematic. Confluency growth assay with (B) H1299 cells and (C) mouse KPC cells. Each value is given as the mean±SD. Western Blot analysis on DNA damage and cell death markers (D) H1299 cells and (E) mouse KPC cells.

FIG. 25 shows FACS analysis of H1299 cells treated with 5-VdU/PINK.

FIG. 26 shows induction of apoptosis by the 5-VdU/PINK combination. Synergistic lethality of 5-VdU and PINK in H1299 cells. (A) Timeline. (B) Percentage of H1299 cells undergoing apoptosis. Each value is given as the mean±SD. (C) Western Blot analysis results of H1299 treated with 5-VdU and 5 μM PINK at different time points.

FIG. 27 shows 5-VdU incorporation in rapidly proliferating cells in vivo. (A) Bone marrow cells, (B) small intestine, and (C) speen tissue.

FIG. 28 shows 5-VdU/PINK Click Chemistry reaction in mouse xenografts. (A) DMSO injected mouse, (B) 5-VdU injected mouse.

FIG. 29 shows 5-VdU/PINK combinatory effects are independent of cellular p53 mutation status. Each value is given as the mean±SD. (A) Treatment schematic, (B) HCT116 p53+/+ cells, (C) isogenic HCT116 p53−/− cell line, (D) Western Blot analysis.

FIG. 30 shows Apotracker™ Green assay images of apoptotic cells in H1299 cells at 72h.

FIG. 31 shows in vivo treatment schematics. (A), experimental group size n=6, and FIG. 7, experimental group size n=3 (B).

FIG. 32 shows incorporation of 5-VdU in mouse xenograft cells as revealed by incubation of tissue slices with PINK. Mouse injected with 5-VdU (B), and control mouse injected with DMSO (A).

### DETAILED DESCRIPTION

Metabolic labeling of nucleic acids without affecting nucleic acid metabolism, the cell cycle, or toxicity to cells and organisms is challenging. For example, chemical modification of nucleic acids in living cells can be sterically hindered by tight packing of packing of bioorthogonal functional groups in chromatin. Provided herein is a dual enhancement strategy for nucleic acid-templated reactions utilizing a fluorogenic intercalating agent capable of undergoing inverse electron-demand Diels-Alder (IEDDA) reactions with DNA or RNA containing one or more types of vinyl-nucleoside. The intercalating agent has a tetrazine-functionalized structure is based on acridine orange. Intercalation the agent induces a close proximity between the tetrazine moiety and the terminal alkene group of vinyl-nucleosides. In addition, the tetrazine quenches fluorescence via photoinduced electron transfer (PET), making the reaction highly fluorogenic.

### Definitions

Unless defined otherwise, all technical and scientific terms used herein have the same meaning as is commonly understood by one of ordinary skill in the art. In the event that there are a plurality of definitions for a term herein, those in this section prevail unless stated otherwise.

The term “Cₓ” followed by a functional group name (e.g., “C₁₋₆ alkyl”) refers to a functional group comprising x carbon atoms (e.g., the term “C₆ alkyl” refers to an alkyl group comprising 6 carbon atom(s) such as hexyl or 2,3-dimethylbutyl). The term “C_{x-y}” followed by a functional group name (e.g., “C₁₋₆ alkyl”) refers to a functional group comprising from x to y (with all individual integers within the range included, including integers x and y) of carbon atoms (e.g., the term “C₁₋₆ alkyl” refers to an alkyl group comprising 1, 2, 3, 4, 5, or 6 carbon atom(s)). The terms “Cₓ” or “C_{x-y}” followed by a hetero functional group name (e.g., “heterocyclyls”) refers to a functional group comprising x or x to y of atoms, inclusive of the heteroatom, respectively (e.g., the term “C₆ heterocyclyl” refers to a ring system comprising 6 ring-forming atoms, where ring-forming atoms includes both ring-forming carbon atoms and heteroatoms). If no “x” or “y” are designated with regards to a functional group, then the broadest range described in these definitions is to be assumed.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z.

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Likewise, it is understood that, in any compound described, all tautomeric forms are also intended to be included.

As used herein, any “R” group(s) such as, but not limited to, R₁ R₂, R₂ₐ, R_{2b}, and R₃ R₄ represent substituents that can be attached to the indicated atom. An R group may be optionally substituted.

Whenever a group is described as being “optionally substituted” or as “an optionally substituted group thereof”, that group may be unsubstituted or substituted with one or more substituents. Likewise, when a group is described as being “unsubstituted or substituted”, if substituted, the substituent(s) may be selected from one or more of the indicated substituents. If no substituents are indicated, it is meant that the indicated “optionally substituted” or “substituted” group may be substituted with one or more substituent group(s) individually and independently selected unless otherwise indicated. Non-limiting examples of possible substituents include alkyl, halogen, alkenyl, alkynyl, alkylidene, aryl, haloalkyl, cycloalkyl, heteroaryl, heterocyclyl, hydroxyl, halide, cyano, amino, thiol, alkoxy, acyl, sulfenyl, sulfinyl, sulfonyl, O-carboxy, C-carboxy, thiocarbonyl, pyridyl, pyrimidinyl, phenyl, as defined herein.

As used herein, “alkyl”, by itself or as part of another group or substituent, refers to a straight or branched hydrocarbon chain that comprises a fully saturated (no double or triple bonds) hydrocarbon group. Typical alkyl groups include, but are in no way limited to, methyl, ethyl, propyl, isopropyl, butyl, isobutyl, t-butyl, pentyl and hexyl. Unless otherwise indicated, the alkyl group may be optionally substituted.

As used herein, “alkenyl”, by itself or as part of another group or substituent, refers to a straight or branched hydrocarbon chain that comprises one or more double bonds. Unless otherwise indicated, the alkenyl group may be optionally substituted.

As used herein, “alkynyl”, by itself or as part of another group or substituent, refers to a straight or branched hydrocarbon chain that comprises one or more triple bonds. Unless otherwise indicated, the alkynyl group may be optionally substituted.

As used herein, “alkylidene”, by itself or as part of another group or substituent, refers to a straight or branched hydrocarbon chain that comprises a fully saturated (no double or triple bonds) hydrocarbon group except that the carbon at the point of attachment forms a double bond with the base molecule. Unless otherwise indicated, the alkylidene group may be optionally substituted.

As used herein, “aryl”, by itself or as part of another group or substituent, refers to a carbocyclic (all carbon) monocyclic or polycyclic aromatic ring system (including fused ring systems where two carbocyclic rings share a chemical bond) that has a fully delocalized pi-electron system throughout all the rings. A ring-forming carbon atom of a monocyclic ring may not be replaced by a ring-forming heteroatom. A ring-forming carbon atom of a fused ring system may be replaced by a ring-forming heteroatom selected from, for example, N, O and S; however, if a fused ring system contains any ring-forming heteroatoms, the ring-forming heteroatoms are not contained in the ring that contains the ring-forming carbon atom that is the point of attachment to the base molecule. Fused polycyclic ring systems include a fused ring system comprising an aromatic ring fused to: (i) one or more aryl rings; ii) one or more cycloalkyl rings; (iii) one or more heterocycloalkyl rings; (iv) one or more heteroaryl rings; or (v) any combination or subcombination of (i), (ii), (iii), and (iv). The point of attachment to the base molecule on an aryl group is a ring-forming carbon atom. For greater clarity, where an aryl group is a fused ring system, the point of attachment to the base molecule on the fused ring system is a ring-forming carbon atom of an aromatic ring of the fused ring system, wherein the aromatic ring does not contain any ring-forming heteroatoms. Typical examples of aryl groups include, but are not limited to, phenyl, naphthyl, anthracyl, phenanthrenyl, indanyl, indenyl, and tetrahydronaphthyl. Unless otherwise indicated, the aryl group may be optionally substituted.

As used herein, “haloalkyl”, by itself or as part of another group or substituent, refers to a subset of the alkyl group wherein one or more of the hydrogen atoms in the alkyl group are replaced by a halogen (e.g., mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. Unless otherwise indicated, the haloalkyl group may be optionally substituted. Similarly, “haloalkenyl”, by itself or as part of another group or substituent, refers a subset of the alkenyl group wherein one or both of the hydrogen atoms in the alkenyl group are replaced by a halogen (e.g., mono-haloalkenyl, di-haloalkenyl). Similarly, “haloalkynyl”, by itself or as part of another group or substituent, refers a subset of the alkynyl group wherein the hydrogen atom in the alkynyl group is replaced by a halogen.

As used herein, “cycloalkyl”, by itself or as part of another group or substituent, refers to a completely saturated (no double or triple bonds) monocyclic or polycyclic hydrocarbon ring system. When composed of two or more rings, the rings may be joined together in a spiro, bridged, or fused fashion wherein at least one ring of the ring system is a hydrocarbon ring wherein all unfused ring-forming carbon atoms are saturated and contains a ring-forming carbon atom that is the point of attachment to the base molecule. A ring-forming carbon atom of a monocyclic ring may not be replaced by a ring-forming heteroatom. A ring-forming carbon atom of a polycyclic ring system may be replaced by a ring-forming heteroatom selected from, for example, N, O and S; however, if a ring system contains any ring-forming heteroatoms, the ring-forming heteroatoms are not contained in the ring that contains the ring-forming carbon atom that is the point of attachment to the base molecule. Fused bicyclic or polycyclic ring systems include a fused ring system comprising a non-aromatic cycloalkyl ring fused to: (i) one or more cycloalkyl rings; (ii) one or more aryl rings; (iii) one or more heterocycloalkyl rings; (iv) one or more heteroaryl rings; or (v) any combination or subcombination of (i), (ii), (iii), and (iv). The point of attachment to the base molecule on a cycloalkyl group is a ring-forming carbon atom. For greater clarity, where a cycloalkyl group is a ring system, the point of attachment to the base molecule on the ring system is a ring-forming carbon atom of a non-aromatic cycloalkyl ring of the fused ring system, wherein the non-aromatic cycloalkyl ring does not contain any ring-forming heteroatoms. Non-limiting examples of cycloalkyl groups include cyclopropyl, cyclobutyl, cyclopentyl, cyclohexyl, cycloheptyl, cyclooctyl, cyclononyl, and cyclodecanyl. Unless otherwise indicated, the cycloalkyl group may be optionally substituted.

As used herein, “heteroalkyl”, by itself or as part of another group or substituent, refers to an alkyl group that contain(s) one or more heteroatoms, that is, an element other than carbon, including but not limited to, N, O, or S.

As used herein, “heteroaryl”, by itself or as part of another group or substituent, refers to a monocyclic or polycyclic aromatic ring system (at least one ring in the system containing a fully delocalized pi-electron system) that contain(s) one or more heteroatoms, that is, an element other than carbon, including but not limited to, N, O, or S. Additionally, any nitrogens in a heterocyclyl may be quaternized. The term “heteroaryl” includes fused ring systems where two rings, such as at least one aryl ring and at least one heteroaryl ring, or at least two heteroaryl rings, share at least one chemical bond. Fused bicyclic or polycyclic ring systems include a fused ring system comprising a heteroaromatic ring fused to: (i) one or more heteroaryl rings; (ii) one or more aryl rings; (iii) one or more cycloalkyl rings; (iv) one or more heterocyclyl rings; or (v) any combination or subcombination of (i), (ii), (iii), (iv) and (v). The point of attachment to the base molecule on a heteroaryl group is a ring-forming atom. For greater clarity, where a heteroaryl group is a fused ring system, the point of attachment to the base molecule on the fused ring system is a ring-forming atom of a heteroaromatic ring of the fused ring system. Non-limiting examples of heteroaryl groups include pyrrolyl, furanyl, furazanyl, thiophenyl, pyrazolyl, benzopyrazolyl, imidazolyl, benzoisoxazolyl isoxazolyl, oxazolyl, isothiazolyl, thiazolyl, triazolyl. Unless otherwise indicated, the heteroaryl group may be optionally substituted.

As used herein, “heterocyclyl” refers to monocyclic or polycyclic ring systems wherein carbon atoms together with heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, O, S, and N. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocyclyl may be quaternized. Fused polycyclic ring systems include a fused ring system comprising a heterocyclyl ring fused to: (i) one or more heteroaryl rings; (ii) one or more aryl rings; (iii) one or more cycloalkyl rings; (iv) one or more heterocyclyl rings; or (v) any combination or subcombination of (i), (ii), (iii), (iv) and (v). The point of attachment to the base molecule on a heterocyclyl group is a ring-forming atom. For greater clarity, where a heterocyclyl group is a fused ring system, the point of attachment to the base molecule on the fused ring system is a ring-forming atom of a heterocyclyl ring of the fused ring system. Examples of such “heterocyclyl” groups include, but are not limited to, dioxanyl, dioxolanyl, oxathianyl, succinimidyl, dioxopiperazinyl, hydantoinyl, trioxanyl, imidazolinyl, imidazolidinyl, isoxazolinyl, isoxazolidinyl, oxazolinyl, oxazolidinyl, oxazolidinonyl, thiazolinyl, thiazolidinyl, morpholinyl, oxiranyl, piperidiny, piperazinyl, pyrrolidinyl, pyrrolidonyl, pyrrolidionyl, 4-piperidonyl, pyrazolinyl, pyrazolidinyl. Unless otherwise indicated, the heterocyclyl group may be optionally substituted.

As used herein, the terms “hydroxy”, by themselves or as part of another group, refer to an alcohol functional group. The alcohol functional group may be depicted as “—OH”.

As used herein, the terms “halogen atom” or “halogen”, by themselves or as part of another group, refer to any one of the radio-stable atoms of column 7 of the Periodic Table of Elements, such as, fluorine, chlorine, bromine and iodine. Fluorine may also be depicted as “F”, “—F” or “fluoro”. Chlorine may also be depicted as “Cl”, “—Cl” or “chloro”. Bromine may also be depicted as “Br”, “—Br” or “bromo”. Iodine may also be depicted as “I”, “—I” or “iodo”.

As used herein, the terms “cyano”, by itself or as part of another group, refers to a nitrile functional group. The nitrile functional group may be depicted as “—CN”.

As used herein, the term “amino”, by itself or as part of another group, refers to an amine functional group. The amine functional group may be depicted as “—NR₂” wherein the R groups may be independently and individually selected from, for example, a hydrogen, a halogen, an alkyl, an alkenyl, an alkynyl, an aryl, a haloalkyl, a cycloalkyl, a heteroaryl, a heterocyclyl, as defined herein. Unless otherwise indicated, the amino group may be optionally substituted.

As used herein, the term “thiol”, by itself or as part of another group, means a thiol functional group. The thiol functional group may be depicted as “—SH”.

As used herein, “alkoxy”, by itself or as part of another group, refers to a substituent containing a single bond to an oxygen atom and that oxygen atom also serves as the point of attachment to the base molecule. The alkoxy group may contain a straight or branched chain. An alkoxy group may be depicted as “—OR” wherein R is, for example, an alkyl, an alkenyl, an alkynyl, an aryl, a haloalkyl, a cycloalkyl, a heteroaryl, a heterocyclyl, as defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. Unless otherwise indicated, the alkoxy group may be optionally substituted.

As used herein, “acyl”, by itself or as part of another group, refers to a substituent connected to the base molecule through a carbonyl group. The acyl group may contain a straight or branched chain. An acyl may be depicted as “—C(═O) R” or “—C(O) R” wherein R is, for example, a hydrogen, a halogen, an alkyl, an alkenyl, an alkynyl, an aryl, a haloalkyl, a cycloalkyl, a heteroaryl, a heterocyclyl, as defined herein. Examples include formyl, acetyl, propanoyl, benzoyl, and acryl. Unless otherwise indicated, the acyl group may be optionally substituted.

As used herein, “sulfenyl”, by itself or as part of another group, refers to a substituent containing a single bond to a sulfur atom and that sulfur atom also serves as the point of attachment to the base molecule. The sulfenyl group may contain a straight or branched chain. The sulfenyl functional group may be depicted as “—SR” wherein R is, for example, an alkyl, an alkenyl, an alkynyl, an aryl, a haloalkyl, a cycloalkyl, a heteroaryl, a heterocyclyl, as defined herein. Unless otherwise indicated, the sulfenyl group may be optionally substituted.

As used herein, “sulfinyl”, by itself or as part of another group, refers to a substituent connected to the base molecule through a sulfur atom which is also doubly bonded to an oxygen atom. The sulfinyl functional group may be depicted as “—S(═O)—R” or “—S(O) R” wherein R can be the same as defined with respect to sulfenyl. Unless otherwise indicated, the sulfinyl group may be optionally substituted.

As used herein “sulfonyl”, by itself or as part of another group, refers to a substituent connected to the base molecule through a sulfur atom which is also doubly bonded to each of two oxygen atoms. The sulfonyl functional group may be depicted as “—SO₂R” wherein R can be the same as defined with respect to sulfenyl. Unless otherwise indicated, the sulfonyl group may be optionally substituted.

As used herein “O-carboxy”, by itself or as part of another group, refers to a substituent containing a carboalkoxy group wherein the point of attachment is on the oxygen atom. The O-carboxy may be depicted as “RC(═O)O—” or “RC(O)O—” or “RCOO—” wherein R is, for example a hydrogen, a halogen, an alkyl, an alkenyl, an alkynyl, an aryl, a haloalkyl, a cycloalkyl, a heteroaryl, a heterocyclyl, as defined herein. Unless otherwise indicated, the O-carboxy group may be optionally substituted.

As used herein, the terms “ester” and “C-carboxy”, by themselves or as part of another group, refer to a substituent containing a carboalkoxy group wherein the point of attachment is on the carbon atom. These may be depicted as “—C(═O) OR” or “—C(O) OR” or “—COOR” wherein R can be the same as defined with respect to O-carboxy. Unless otherwise indicated, the C-carboxy group may be optionally substituted.

As used herein, the term “thiocarbonyl”, by itself or as part of another group, refers to a functional group similar to the acyl group wherein the oxygen atom is replaced with a sulfur atom. A thiocarbonyl may be depicted as “—C(═S)R” or “—C(S)R”, wherein the R can be the same as defined with respect to the acyl group. Unless otherwise indicated, the thiocarbonyl group may be optionally substituted.

As used herein, the term “pyridyl”, by itself or as part of another group, refers to a substituent that consists of a pyridine molecule wherein one of the carbon atoms serves as the point of attachment (and has one fewer hydrogen atoms). The pyridyl group is considered part of the heteroaryl group. Unless otherwise indicated, the pyridyl group may be optionally substituted.

As used herein, the term “pyrimidinyl”, by itself or as part of another group, refers to a substituent that consists of a pyrimidine molecule wherein one of the carbon atoms serves as the point of attachment (and has one fewer hydrogen atoms). The pyrimidinyl group is a considered part of the heteroaryl group. Unless otherwise indicated, the pyrimidinyl group may be optionally substituted.

As used herein, the term “phenyl”, by itself or as part of another group, refers to a substituent that consists of a benzene molecule wherein one of the carbon atoms serves as the point of attachment (and has one fewer hydrogen atoms). The phenyl group is considered part of the aryl group. Unless otherwise indicated, the phenyl group may be optionally substituted.

As used herein, the abbreviations for any protective groups, amino acids and other compounds, are, unless indicated otherwise, in accord with their common usage, recognized abbreviations, or the IUPAC-IUB Commission on Biochemical Nomenclature. Terms and symbols used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The term “nucleoside” is used herein in its ordinary sense as understood by those skilled in the art, and refers to a compound composed of an optionally substituted pentose moiety or modified pentose moiety attached to an optionally substituted heterocyclic base or tautomer thereof, such as attached via the 9-position of a purine-base or the 1-position of a pyrimidine-base. Examples include, but are not limited to, a ribonucleoside comprising a ribose moiety and a deoxyribonucleoside comprising a deoxyribose moiety. A “nucleoside” is a monomer that can have a substituted base and/or sugar moiety. As used herein, a “vinyl-nucleoside” refers to a nucleoside analogue having a vinyl group conjugated to the base moiety. Example vinyl-nucleosides are disclosed in WO2015197655, the entire content of which is incorporate herein by reference. Additionally, a nucleoside can be incorporated into larger DNA and/or RNA polymers and oligomers.

The term “nucleotide” is used herein in its ordinary sense as understood by those skilled in the art, and refers to a nucleoside having a phosphate ester bound to the pentose moiety, for example, at the 5′-position.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having or at risk of having an adhesion. Subjects may be human, but also include other mammals, particularly those mammals useful as laboratory models for human disease, e.g. mouse, rat, etc.

The term “diagnosis” is used herein to refer to the identification of a pathological state, disease or condition, such as the identification and characterization of cancer or tumour.

“Pharmaceutically acceptable salt” as used herein includes, for example, salts that have the desired pharmacological activity of the parent compound (salts which retain the biological effectiveness and/or properties of the parent compound and which are not biologically and/or otherwise undesirable). Compounds as described herein having one or more functional groups capable of forming a salt may be, for example, formed as a pharmaceutically acceptable salt. Compounds containing one or more basic functional groups may be capable of forming a pharmaceutically acceptable salt with, for example, a pharmaceutically acceptable organic or inorganic acid.

“Pharmaceutically acceptable excipient” or “Pharmaceutically acceptable carrier” means an excipient or carrier that is useful in preparing a pharmaceutical composition that is generally safe, non-toxic, and desirable, and includes excipients or carriers that are acceptable for veterinary use as well as for human pharmaceutical use. Such excipients can be solid, liquid, semisolid, or, in the case of an aerosol composition, gaseous.

The term “pharmaceutically acceptable”, as they refer to compositions, carriers, diluents and reagents, represent that the materials are capable of administration to or upon a human without the production of undesirable physiological effects to a degree that would prohibit administration of the composition.

“In combination with” refer, in certain embodiments, to the concurrent administration to a patient of a first therapeutic and a second therapeutic. When administered in combination, each component can be administered at the same time or sequentially in any order at different points in time. Thus, each component can be administered separately but sufficiently closely in time so as to provide the desired therapeutic effect.

### Intercalating Agent

The intercalating agent provided herein is an acridine-tetrazine conjugate compound. As used herein “acridine moiety” refers to a portion of compound having core chemical structure

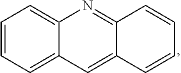 [SMILES: c1ccc2nc3ccccc3cc2c1]

and “tetrazine moiety” refers to a portion of compound having core chemical structure

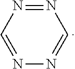

Methods and processes for conjugating an acridine moiety to a tetrazine moiety is disclosed for example in Yang et al. (Metal-Catalyzed One-Pot Synthesis of Tetrazines Directly from Aliphatic Nitriles and Hydrazine, Angewandte Chemie International Edition, vol. 51, issue 21, pp 5222-5225 May 21, 2012), the entire content of which is incorporated herein by reference.

In some embodiments, the compound has one of the following formulas (brackets indicate optional groups):

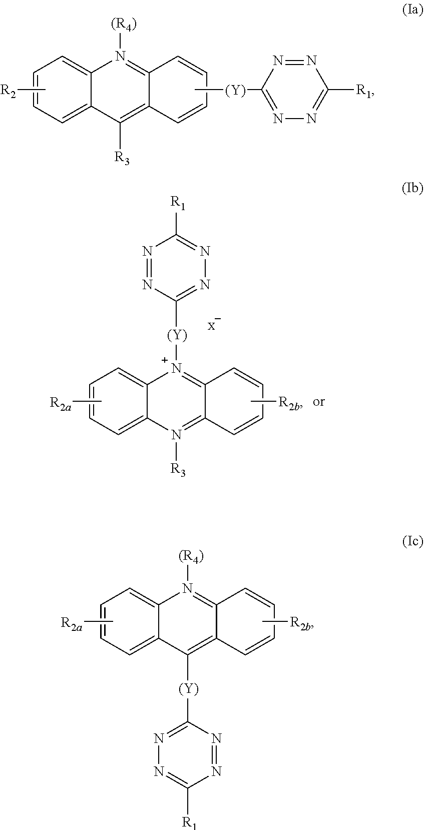

In some embodiments, the compound has formulas Ia or Ic, and R₄ is absent. In other embodiments, the compound has formulas Ia or Ic, and R₄ is present resulting in acridinium salt formation. In some embodiments, the compound has formula Ib and is a salt, where X is the counter ion. Salts of the present compound include, but are not limited to chlorine salts, fluorine salts, bromine salts, iodine salts, or trifluoroacetate (TFA) salts; where X is Cl, F, Br, I, or TFA. In some embodiments, the salts of the present compounds are pharmaceutically acceptable salts.

In some embodiments, R₁ is H, haloalkyl, an optionally substituted haloalkyl, alkyl, an optionally substituted alkyl, alkenyl, an optionally substituted alkenyl, alkynyl, an optionally substituted alkynyl, cycloalkyl, an optionally substituted cycloalkyl, aryl, an optionally substituted aryl, heteroaryl, an optionally substituted heteroaryl, or heterocyclyl, or an optionally substituted heterocyclyl.

In one embodiment, R₁ is H, C₁₋₆ alkyl, or an optionally substituted aryl or heteroaryl. In one embodiment, R₁ is, methyl, ethyl, propyl, isopropyl, a C₄ alkyl, a C₅ alkyl, or a C₆ alkyl; preferably, R₁ is methyl. In one embodiment, R₁ is pyridyl, an optionally substituted pyridyl, pyrimidinyl, an optionally substituted pyrimidinyl, C₆₋₁₀ aryl, or an optionally substituted C₆₋₁₀ aryl. In one embodiment, R₁ is C₆ aryl or phenyl.

In some embodiments, R₂, R₂ₐ, R_{2b}, and R₃ are each independently H, hydroxy, hydroxyl, halogen, haloalkyl, an optionally substituted haloalkyl, cyano, amino, an optionally substituted amino, thiol, an optionally substituted thiol, alkyl, an optionally substituted alkyl, alkenyl, an optionally substituted alkenyl, alkynyl, an optionally substituted alkynyl, cycloalkyl, an optionally substituted cycloalkyl, aryl, an optionally substituted aryl, heteroaryl, an optionally substituted heteroaryl, heterocyclyl, an optionally substituted heterocyclyl, alkoxy, an optionally substituted alkoxy, sulfenyl, an optionally substituted sulfenyl, acyl, an optionally substituted acyl, sulfinyl, an optionally substituted sulfinyl, sulfonyl, an optionally substituted sulfonyl, O-carboxy, an optionally substituted O-carboxy, ester, an optionally substituted ester, thiocarbonyl, or an optionally substituted thiocarbonyl.

In one embodiment, R₂, R₂ₐ, and R_{2b}, are each independently H, alkyl, an optionally substituted alkyl, amino, an optionally substituted amino, C₆₋₁₀ cycloalkyl, an optionally substituted C₆₋₁₀ cycloalkyl, C₆₋₁₀ aryl, an optionally substituted C₆₋₁₀ aryl, heteroaryl, an optionally substituted heteroaryl, heterocyclyl, or an optionally substituted heterocyclyl. In one embodiment, R₂, R₂ₐ, and R_{2b}, are each independently H, C₁₋₆ alkyl, optionally substituted C₁₋₆ alkyl, amino, optionally substituted amino, optionally substituted heterocyclyl, or optionally substituted heterocyclyl that forms a ring with the acridine moiety.

In one embodiment, R₂, R₂ₐ, and/or R_{2b} is a forms a julolidine group with the acridine moiety as shown below, which may be further optionally substituted with one or more groups:

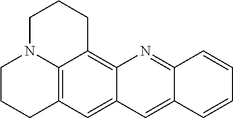 [SMILES: c1ccc2nc3c4c5c(cc3cc2c1)CCCN5CCC4]

In one embodiment, the compound has formula (Ia), and R₂ is amino substituted with C₁₋₆ alkyl, or an optionally substituted heterocyclyl that forms a ring with the acridine moiety. In one embodiment, the compound has formula (Ia), and R₂ is amino substituted with C₁₋₆ alkyl, preferably amino substituted with one or more C₁₋₃ alkyl, more preferably dimethylamine. In one embodiment, the compound has formula (Ia), and R₂ is an optionally substituted heterocyclyl that forms a ring with the acridine moiety, preferably a julolidine group with the acridine moiety.

In one embodiment, the compound has formula (Ib) or (Ic), and R₂ₐ and R_{2b} are independently H, amino substituted with C₁₋₆ alkyl, or optionally substituted heterocyclyl that forms a ring with the acridine moiety. In one embodiment, the compound has formula (Ib) or (Ic), and one of R₂ₐ and R_{2b} is amino substituted with C₁₋₆ alkyl, preferably amino substituted with one or more C₁₋₃ alkyl, more preferably dimethylamine. In one embodiment, the compound has (Ib) or (Ic), and R₂ₐ and R_{2b} are both amino substituted with C₁₋₆ alkyl, preferably amino substituted with one or more C₁₋₃ alkyl, more preferably dimethylamine. In one embodiment, the compound has formula (Ib) or (Ic), and one of R₂ₐ and R_{2b} is an optionally substituted heterocyclyl that forms a ring with the acridine moiety, preferably a julolidine group with the acridine moiety. In one embodiment, the compound has formula (Ib) or (Ic), and R₂ₐ and R_{2b} are both an optionally substituted heterocyclyl that forms a ring with the acridine moiety, preferably a julolidine group with the acridine moiety.

In one embodiment, R₃ is H, alkyl, an optionally substituted alkyl, heteroalkyl, an optionally substituted heteroalkyl, aryl, an optionally substituted aryl, heteroaryl, or an optionally substituted heteroaryl. In one embodiment, R₃ is H, optionally substituted C₁₋₆ alkyl, C₁₋₆ heteroalkyl, optionally substituted 5 or 6 membered heteroaryl, or optionally substituted C₆₋₁₀ aryl.

In some embodiments, R₄ is present and is H, haloalkyl, an optionally substituted haloalkyl, alkyl, an optionally substituted alkyl, alkenyl, an optionally substituted alkenyl, alkynyl, an optionally substituted alkynyl, cycloalkyl, an optionally substituted cycloalkyl, aryl, an optionally substituted aryl, heteroaryl, an optionally substituted heteroaryl, heterocyclyl, or an optionally substituted heterocyclyl. In one embodiment, R₄ is present and is H or C₁₋₆ alkyl; preferably H or CH₃.

In some embodiments, the compound has an optional linker (Y). In some embodiments, the optional linker is absent and the “acridine moiety” is directly conjugated to “tetrazine moiety”. In some embodiments, the compound has a linker. In one embodiment, Y is alkyl, an optionally substituted alkyl, heteroalkyl, an optionally substituted heteroalkyl, aryl, an optionally substituted aryl, heteroaryl, an optionally substituted heteroaryl, alkenyl, an optionally substituted alkenyl, alkynyl, an optionally substituted alkynyl, alkylthio, or an optionally substituted alkylthio. In one embodiment, Y is optionally substituted C₁₋₆ alkyl, C₁₋₆ optionally substituted heteroalkyl, optionally substituted C₆₋₁₀ aryl, optionally substituted 5 or 6 membered heteroaryl, optionally substituted C₁₋₆ alkenyl, optionally substituted C₁₋₆ alkynyl, optionally substituted C₁₋₆ alkylthio. In one embodiment, Y is C₁₋₃ alkyl, or C₁₋₃ alkylthio.

The compounds or salts disclosed herein exclude:

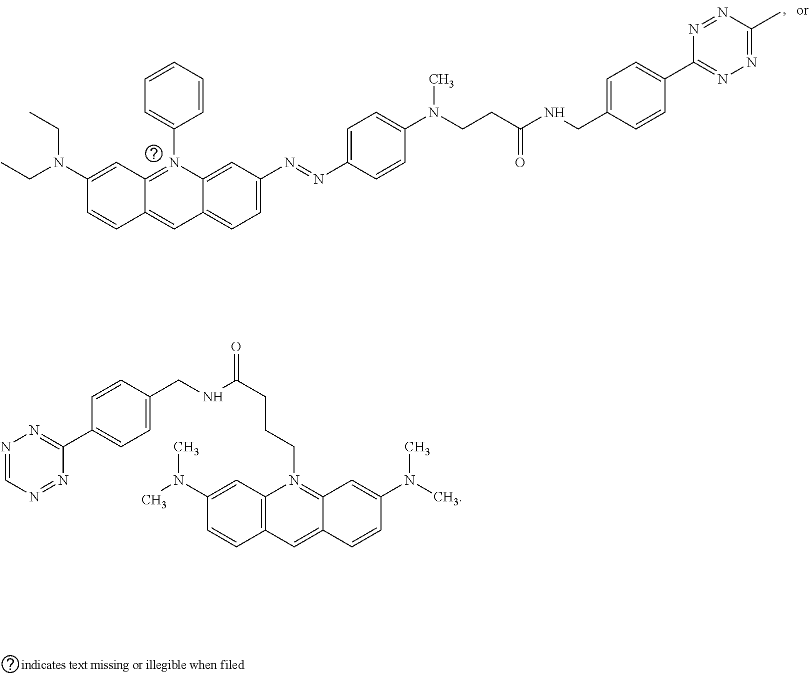

In some embodiments, R₂, R₂ₐ, R_{2b}, and/or Y are at position 3 or 6. In some embodiments, the compound has one of the following formulas (brackets indicate optional groups):

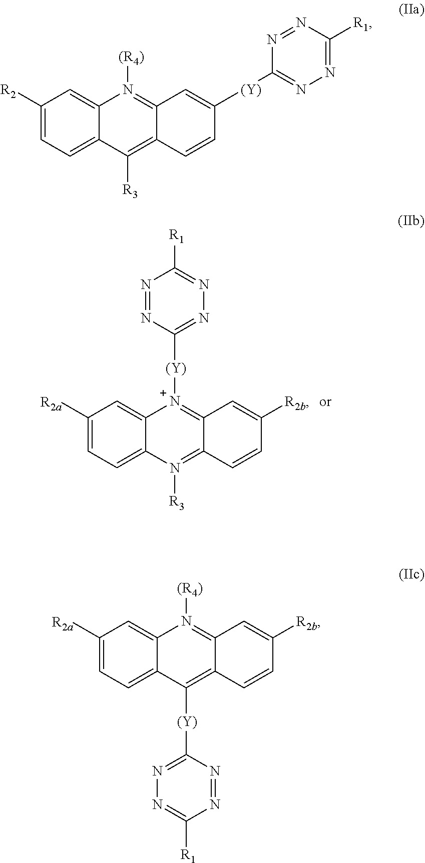

where R₁, R₂, R₂ₐ, R_{2b}, R₃, R₄, and Y are as described above.

In some embodiments, the compound has formula IIIb:

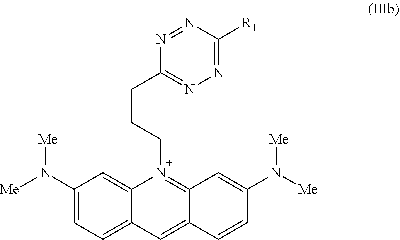

and R₁ is as described above.

Exemplary intercalating agents of the present disclosure include, but are not limited to the below compounds, and their corresponding salts:

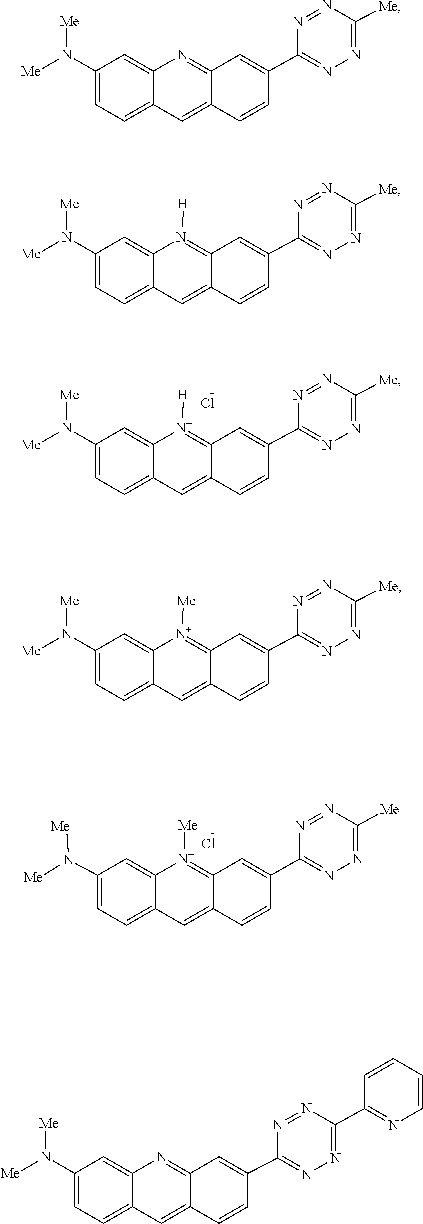

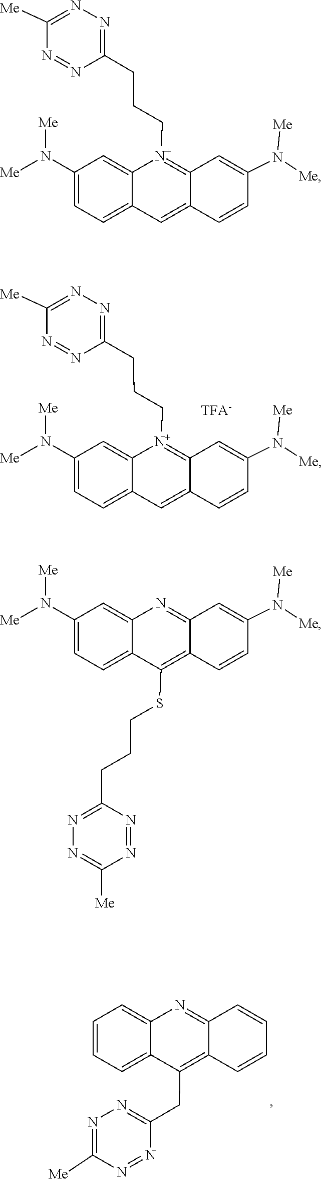 [SMILES: Cc1nnc(CCC[n+]2c3cc(N(C)C)ccc3cc3ccc(N(C)C)cc32)nn1 ; Cc1nnc(CCC[n+]2c3cc(N(C)C)ccc3cc3ccc(N(C)C)cc32)nn1 ; Cc1nnc(CCCSc2c3ccc(N(C)C)cc3nc3cc(N(C)C)ccc23)nn1 ; Cc1nnc(Cc2c3ccccc3nc3ccccc23)nn1]

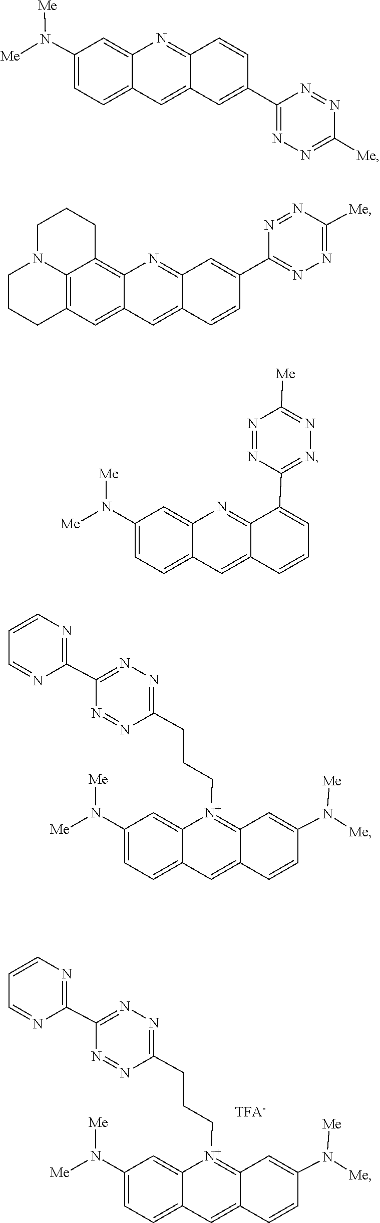 [SMILES: Cc1nnc(-c2ccc3nc4cc(N(C)C)ccc4cc3c2)nn1 ; Cc1nnc(-c2ccc3cc4cc5c6c(c4nc3c2)CCCN6CCC5)nn1 ; Cc1nnc(-c2cccc3cc4ccc(N(C)C)cc4nc23)nn1 ; CN(C)c1ccc2cc3ccc(N(C)C)cc3[n+](CCCc3nnc(-c4ncccn4)nn3)c2c1 ; CN(C)c1ccc2cc3ccc(N(C)C)cc3[n+](CCCc3nnc(-c4ncccn4)nn3)c2c1]

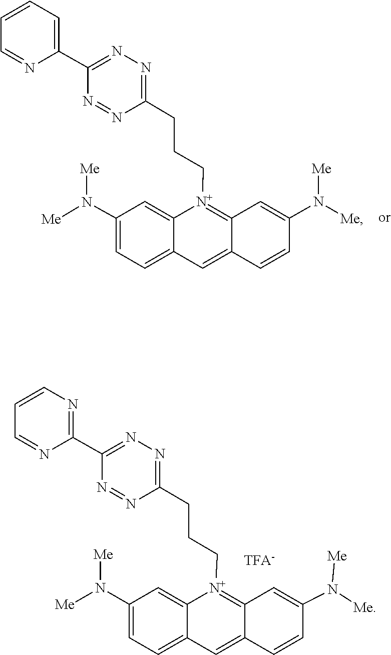 [SMILES: CN(C)c1ccc2cc3ccc(N(C)C)cc3[n+](CCCc3nnc(-c4ccccn4)nn3)c2c1 ; "or" ; CN(C)c1ccc2cc3ccc(N(C)C)cc3[n+](CCCc3nnc(-c4ncccn4)nn3)c2c1]

In some embodiments, the compounds or salts described herein are probes. In one embodiment, the probes are for labelling nucleic acid. In one embodiment, the probes are for labelling a DNA molecule. In one embodiment, the probes are for labelling a RNA molecule. In some embodiments, the compounds or salts described herein are in vivo probes. In some embodiments, the compounds or salts described herein are in vitro probes. In some embodiments, the compounds or salts described herein are for identifying or quantifying cell growth and/or replication.

### Nucleic Acid Labelling

In some embodiments, the acridine-tetrazine conjugate compounds or salts provided herein are for detecting nucleic acid containing a vinyl-nucleoside. In one embodiment, the compounds or salts provided herein are for detecting DNA containing a vinyl-nucleoside. In one embodiment, the compounds or salts provided herein are for detecting RNA containing a vinyl-nucleoside Exemplary vinyl-nucleosides are disclosed in WO2015197655, the entire content of which is incorporated herein by reference. In some embodiments, the compounds or salts provided herein are for detecting nucleic acid containing one or more of 5-vinyl-2′-deoxyuridine (VdU), 5-vinyl-2′-deoxycytidine (VdC), 7-vinyl-7-deaza-2′-deoxyadenosine (VdA), 7-vinyl-7-deaza-2′-deoxyguanosine (VdG). 5-vinyluridine (VU), 5-vinylcytidine (VC), 7-vinyl-7-deazaadenosine (VA), and 7-vinyl-7-deaza-2′-deoxyguanosine (VG). In one embodiment, the vinyl-nucleoside is VdU and/or VdA. In one embodiment, the vinyl-nucleoside is VU.

For nucleic acid labelling, an organism, tissue, or cell is first treated or brought into contact with one or more vinyl-nucleosides, which is incorporated into the DNA and/or RNA molecules of the organism, tissue, or cell. The acridine-tetrazine conjugate compound provided herein is then introduced, which conjugates with the incorporated vinyl-nucleosides to create a fluorescently active moiety. In some embodiments, a method of detecting the nucleic acids containing the vinyl-nucleoside comprise a) contacting the cell or tissue with the compound or salt provided herein, and b) exposing the cell or tissue of a) to light. In one embodiment, the method is conducted in vitro. In another embodiment, the method is conducted in vivo. In some embodiments, a method of detecting the nucleic acids containing the vinyl-nucleoside is conducted in vivo by a) administering an organism with the compound or salt provided herein, and b) exposing the a target area or excised tissue to light.

In some embodiments, a kit for detection of nucleic acid in a cell or tissue is provided for use in vitro or in vivo. The kit comprises one or more vinyl-nucleosides, and the compound or salt as provided herein. In one embodiment, the kit comprises one or more of 5-vinyl-2′-deoxyuridine (VdU), 5-vinyl-2′-deoxycytidine (VdC), 7-vinyl-7-deaza-2′-deoxyadenosine (VdA), 7-vinyl-7-deaza-2′-deoxyguanosine (VdG). 5-vinyluridine (VU), 5-vinylcytidine (VC), 7-vinyl-7-deazaadenosine (VA), and 7-vinyl-7-deaza-2′-deoxyguanosine (VG). In one embodiment, the kit comprises one or more of VdU, VdC, VdA, and VdG. In one embodiment, the kit comprises VdU and/or VdA. In one embodiment, the kit comprises one or more of VU, VC, VA, and VG. In one embodiment, the kit comprises VU.

In some embodiments, nucleic acid labelling using the acridine-tetrazine conjugate compounds or salts provided herein is for diagnosis of cancer. In one embodiment, diagnosis is for tumour growth progression. In some embodiments, nucleic acid labelling using the acridine-tetrazine conjugate compounds or salts provided herein is for cell cycle analysis. In one embodiment, the acridine-tetrazine conjugate compounds or salts together with the one or more vinyl-nucleosides are markers of DNA synthesis in cell cycle analysis. In one embodiment, the acridine-tetrazine conjugate compounds or salts together with the one or more vinyl-nucleosides are S-phase markers in cell cycle analysis.

### Therapeutic Effects

Vinyl-nucleosides are non-toxic nucleoside analogues. However, when an acridine-tetrazine conjugate compound is conjugated to a vinyl-nucleoside, toxicity results.

In some embodiments, a method for treating cancer is provided with an acridine-tetrazine conjugate compound or salt. A subject that has been previously administered with one or more vinyl-nucleosides is then administered with the compound or salt described herein.

In some embodiments, a pharmaceutical composition comprising the acridine-tetrazine conjugate compound or salt and a acceptable pharmaceutical carrier. In some embodiments, the acridine-tetrazine conjugate compound or salt is used in the manufacture of a medicament for the treatment of cancer. In some embodiments, the acridine-tetrazine conjugate compound or salt, or the a pharmaceutical composition comprising the acridine-tetrazine conjugate compound or salt is used in the treatment of cancer. In some embodiments, the acridine-tetrazine conjugate compound or salt, or the a pharmaceutical composition comprising the acridine-tetrazine conjugate compound or salt is used in combination with one or more vinyl-nucleosides in the treatment of cancer. In one embodiment, the acridine-tetrazine conjugate compound or salt is for administration in sequence after administration of one or more vinyl-nucleosides.

In one embodiment, the acridine-tetrazine conjugate compound or salt is for administration in sequence after administration of one or more of 5-vinyl-2′-deoxyuridine (VdU), 5-vinyl-2′-deoxycytidine (VdC), 7-vinyl-7-deaza-2′-deoxyadenosine (VdA), 7-vinyl-7-deaza-2′-deoxyguanosine (VdG). 5-vinyluridine (VU), 5-vinylcytidine (VC), 7-vinyl-7-deazaadenosine (VA), and 7-vinyl-7-deaza-2′-deoxyguanosine (VG). In one embodiment, the acridine-tetrazine conjugate compound or salt is for administration in sequence after administration of one or more of VdU, VdC, VdA, and VdG. In one embodiment, the acridine-tetrazine conjugate compound or salt is for administration in sequence after administration of VdU and/or VdA. In one embodiment, the acridine-tetrazine conjugate compound or salt is for administration in sequence after administration of one or more of VU, VC, VA, and VG. In one embodiment, the the acridine-tetrazine conjugate compound or salt is for administration in sequence after administration of VU. In one embodiment, the acridine-tetrazine conjugate compound or salt is for administration in sequence after administration of one or more vinyl-nucleosides.

In one embodiment, the acridine-tetrazine conjugate compound or salt is for administration in combination with administration of one or more of 5-vinyl-2′-deoxyuridine (VdU), 5-vinyl-2′-deoxycytidine (VdC), 7-vinyl-7-deaza-2′-deoxyadenosine (VdA), 7-vinyl-7-deaza-2′-deoxyguanosine (VdG). 5-vinyluridine (VU), 5-vinylcytidine (VC), 7-vinyl-7-deazaadenosine (VA), and 7-vinyl-7-deaza-2′-deoxyguanosine (VG). In one embodiment, the acridine-tetrazine conjugate compound or salt is for administration in combination with administration of one or more of VdU, VdC, VdA, and VdG. In one embodiment, the acridine-tetrazine conjugate compound or salt is for administration in combination with administration of VdU and/or VdA. In one embodiment, the acridine-tetrazine conjugate compound or salt is for administration in combination with administration of one or more of VU, VC, VA, and VG. In one embodiment, the acridine-tetrazine conjugate compound or salt is for administration in combination with administration of VU.

### EXAMPLES

The following examples illustrate certain embodiments and are not intended to limit the embodiments described elsewhere in this disclosure.

### Example 1-Kinetic and Fluorogenic Enhancement Strategy for Labeling of Nucleic Acids

Chemical modification of nucleic acids in living cells can be sterically hindered by tight packing of bioorthogonal functional groups in chromatin. To address this limitation, a dual enhancement strategy was developed for nucleic acid-templated reactions utilizing a fluorogenic intercalating agent capable of undergoing inverse electron-demand Diels-Alder (IEDDA) reactions with DNA containing 5-vinyl-2′-deoxyuridine (VdU) or RNA containing 5-vinyl-uridine (VU). Reversible high-affinity intercalation of a novel acridine-tetrazine conjugate “PINK” (K_{D}=5±1 μM) increases the reaction rate of tetrazine-alkene IEDDA on duplex DNA by 60,000-fold (590 M⁻¹ s⁻¹) as compared to the non-templated reaction. At the same time, loss of tetrazine-acridine fluorescence quenching renders the reaction highly fluorogenic and detectable under no-wash conditions. This strategy enables live-cell dynamic imaging of acridine-modified nucleic acids in dividing cells.

Chemoselective modification reactions of biomolecules provide indispensable tools for biological and biomedical research.^{[1-4]} In particular, bioorthogonal labeling reactions for chemical biology have been successfully developed for sequence recognition,^{[5-7]} sensitive detection of viral infection^{[8-10]}, protein crosslinking,^{[11]} elucidation of metabolic pathways^{[12]} and probing drug resistance and sensitivity mechanisms.^{[13,14]} Such bioorthogonal methodologies were originally designed for proteins or carbohydrates and subsequently applied to nucleic acids.^{[15-18]} These include copper-catalyzed or strain-promoted azide-alkyne cycloadditions (CuAAC/SPAAC), inverse electron-demand Diels-Alder (IEDDA) reactions between alkenes and tetrazines, and 1,3-dipolar “photoclick” reactions of tetrazoles.^{[19,20]} Despite such progress in vitro, examples of nucleic acids labeling in vivo remain scarce and are either limited to single-stranded RNA^{[21-25]} or rely on membrane permeabilization and/or and phototoxic methods to achieve efficient reactions within the tightly packed double-stranded (ds) DNA of native chromatin.^{[26-28]} In addition, previously reported tetrazine-alkene bioorthogonal reactions for cellular nucleic acids were too slow (0.02-0.42 M⁻¹ s⁻¹) to visualize highly dynamic processes in living cells.^{[24,29-31]}

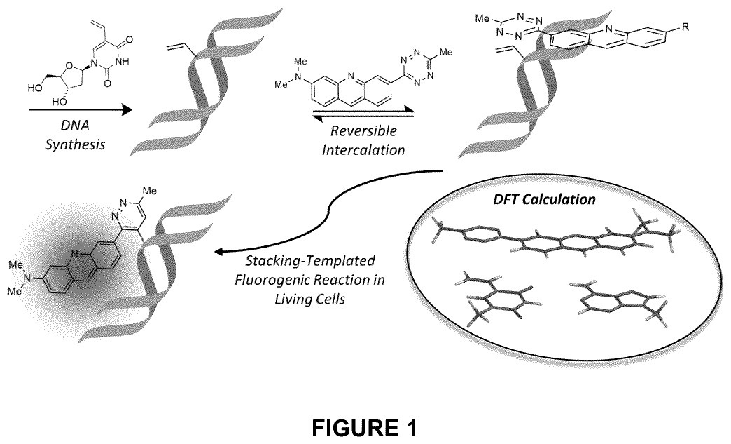

To address these limitations, an intercalating agent was developed based on the Sondheimer diyne that reacts with two azide groups via tandem SPAAC crosslinking reactions.^{[32,33]} This approach is applicable to the modification of dsDNA and RNA in living cells, but the high background fluorescence of the unreacted, fluorescent labeling reagents prevented live-cell imaging. Probe for Imaging Nucleosidic AlKene groups (“PINK”) combines a fluorescent intercalating agent^{[34]} with a tetrazine that serves both as a bioorthogonal functional group and fluorescence quencher.^{[35,36]} PINK is essentially non-fluorescent and exhibits reversible, intercalative “scanning” of the duplex DNA until it encounters a vinyl-modified nucleotide, whereupon it undergoes a highly rapid and fluorogenic IEDDA reaction (FIG. 1). This approach provides the first “mix and measure” fluorogenic assay for DNA replication in living cells.

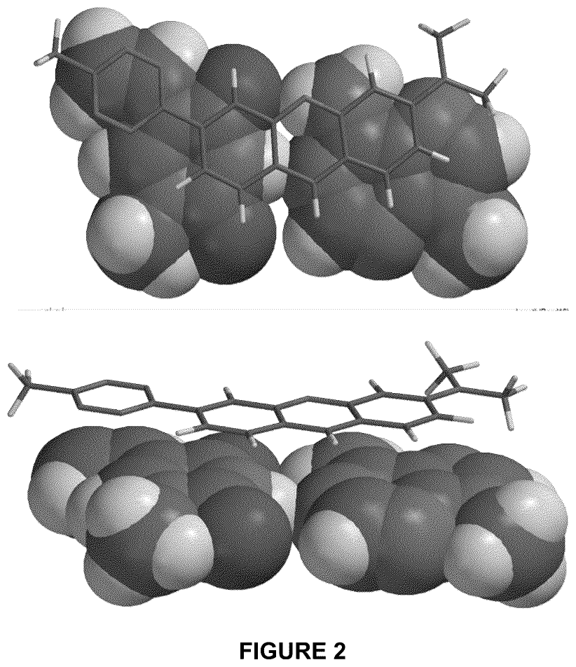

PINK was designed according to crystallographic data and density functional theory (DFT) calculations. A crystal structure of 3,6-bis(dimethylamino) acridine (acridine orange) bound to a CpG dimer revealed co-facial alignment between a dimethylamino substituent and the 5-position of the pyrimidine nucleobase.^{[37]} The 3-position of acridine orange therefore selected for installing a tetrazine moiety while maintaining the dimethylamino group at the 6-position. As previously observed for aryl tetrazines,^{[38,39]} DFT calculations predicted PINK to be a planar molecule (FIG. 1). A model of PINK pi stacked on a VdU-dA base pair was generated by DFT geometry optimization (LSDA/pBP86/DN**) of PINK, flowed by manual docking of PINK and a DFT-geometry-optimized VdU-dA base pair according to the pi-stacking alignment observed in a crustal structure of acridine orange intercalated into a CpG dimer (FIG. 2). Upon intercalation, this co-planarity should result in co-facial alignment of the 5-vinyl and tetrazine groups to facilitate a highly rapid reaction between PINK and VdU residues (FIG. 2).

The synthesis of PINK commenced from aniline 1, which cleanly underwent a Buchwald-Hartwig cross coupling reaction with aryl bromide 2 (Scheme 1).^{[40]} The resulting secondary amine spontaneously cyclized upon contact with mildly Lewis-acidic silica gel to furnish the new acridine nitrile 3 in a 64% isolated yield. Subsequent installation of the asymmetric methyl tetrazine was accomplished by reaction with hydrazine, Zn (II) and acetonitrile followed by oxidation with NaNO₂ to give PINK (4) as a red solid in 18% total yield over four steps.^{[41]}

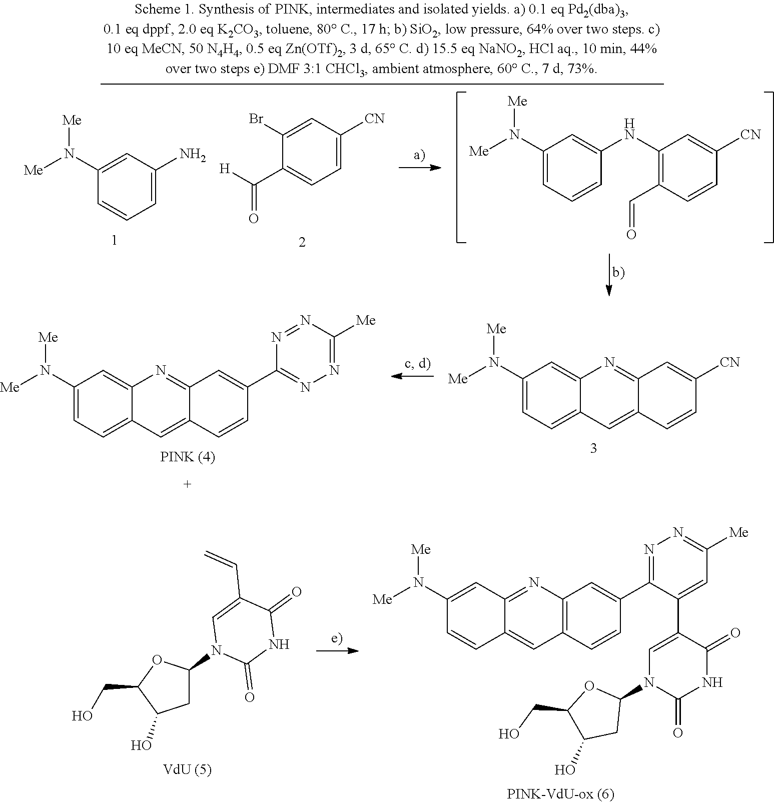

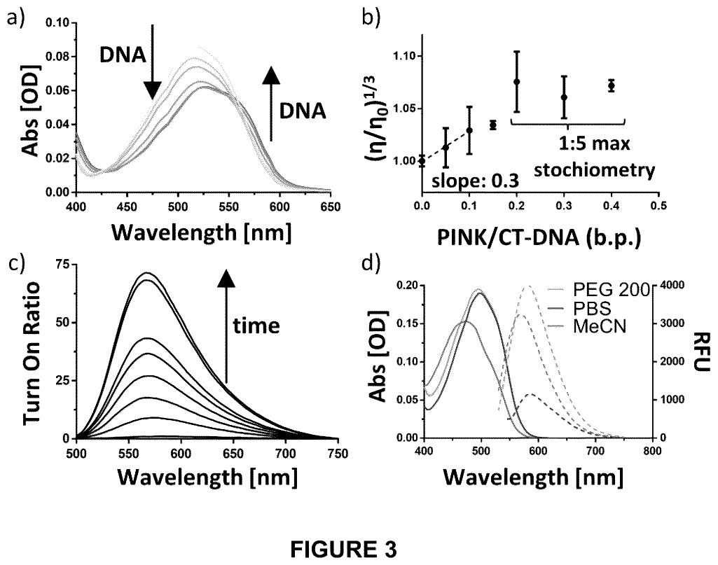

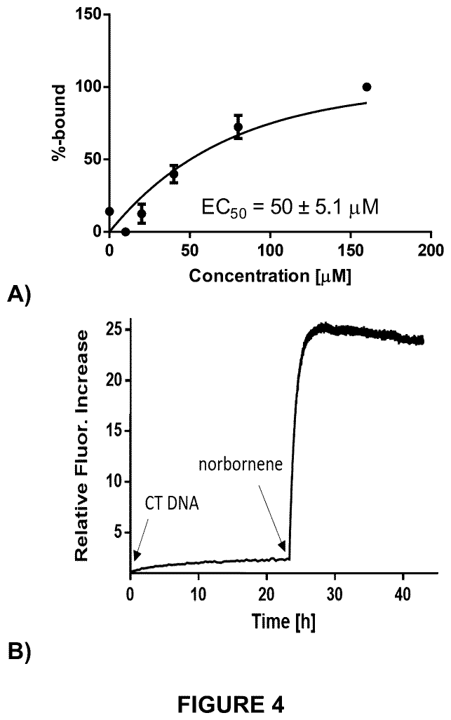

To evaluate the ability of PINK (4) to undergo reversible intercalation into duplex DNA, the absorbance of PINK was monitored upon addition of calf-thymus (CT) DNA. Consistent with intercalation,^{[42-44]} a bathochromic shift in PINK absorbance was observed upon addition of CT DNA (FIG. 3, a). To further evaluate the binding mode, viscosity measurements of concentrated DNA solutions were conducted in presence of increasing amounts of PINK. In accordance with the hypothesis of Lerman for DNA intercalating agents, PINK caused increased viscosity (n) of the solution and exhibited a 1:5 stoichiometry between PINK and DNA base pairs (FIG. 3, b). Using this stoichiometry and titrations conducted using the absorbance changes of PINK with increasing DNA concentrations, an apparent dissociation constant (K_{D})=5.1±1.0 μM (FIG. 3, a; and FIG. 4, A) was estimated. By taking the maximum binding stoichiometry of PINK into account (1 molecule per 5 base pairs; see FIG. 1b), an apparent dissociation constant K_{D} can be calculated according to K_{D}=[(unbound PINK)*(unbound DNA binding sites)]/(PINK-bound binding sites). At the EC₅₀ value and taking stoichiometry into account, this simplifies to K_{D}=[unbound DNA binding sites]=[(EC₅₀)/5]−[unbound PINK]=[(50±5.1 μM)/5]−[5 μM]=5.1±1.0 μM. This DNA affinity is similar to those of other acridine derivatives,^{[46-48]} indicating that the tetrazine moiety of PINK does not interfere with DNA binding. Extended incubation times of PINK with unmodified DNA resulted in little-to-no degradation of PINK (FIG. 4, B), supporting the potential for chemoselective reactions between PINK and VdU-containing DNA.

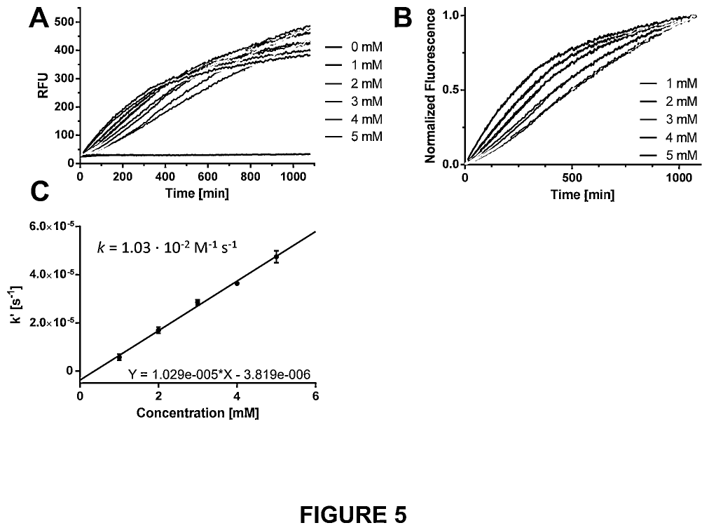

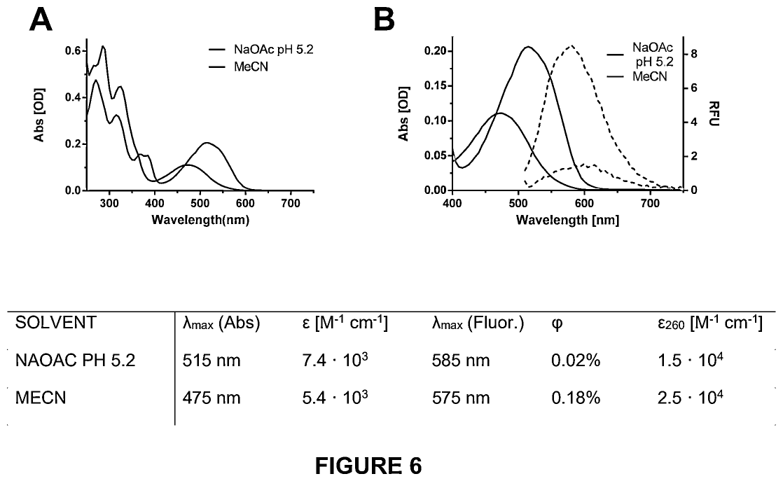

PINK (4) undergoes a highly fluorogenic and regioselective IEDDA reaction with VdU (5) to give “PINK-VdU-ox” (6) as a single isomer in a 76% isolated yield after 7d at 60° C. (Scheme 1). The slow rate of this non-templated reaction (Kapp=0.01 M⁻¹ s⁻¹, FIG. 5) is due to the combination of an unstrained terminal alkene and unactivated tetrazine.^{[49]} Over the course of the reaction, a ˜100-fold increase in fluorescence was observed (FIG. 3, c). With a quantum yield (φ_{F})<0.2%, PINK (4) is essentially non-fluorescent in both aqueous and aprotic solvents (FIG. 6).

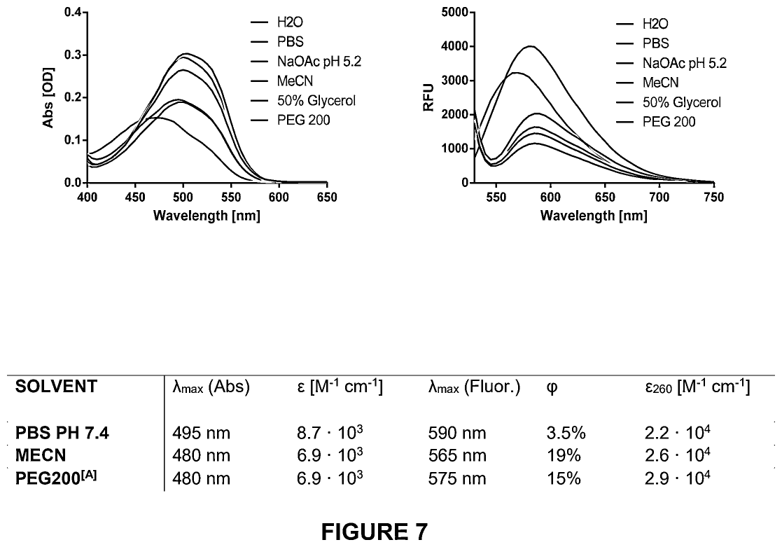

In contrast, the PINK-VdU reaction product (6) exhibits a φ_{F}=3.5-19%, depending on the solvent used (FIG. 3, d; FIG. 7). Highly viscous solvents, including glycerol and polyethylene glycol (PEG 200) induced higher fluorescence of 6, likely by restricting bond rotation and thus suppressing formation of a non-emissive, twisted intramolecular charge transfer (TICT) state.^{[50]}

TABLE 1
Investigated deoxy oligomers. Structures of non-canonical nucleotides
(V = VdU; X = PINK-VdU-ox).

| Name | Sequence | Mol. Weight [g/mol] |
|---|---|---|
| ODN1 | 5′-GCCGCAACGVGCAACGCCG-3′ | 5795.80 |
| compl1 | 5′-CGGCGTTGCACGTTGCGGC-3′ | 5836.80 |
| ODN1_PINK | 5′-GCCGCAACGXGCAACGCCG-3′ | 6082.14 |

V = 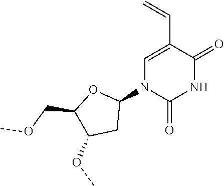
X =

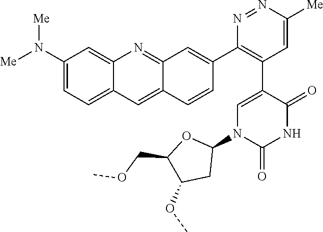

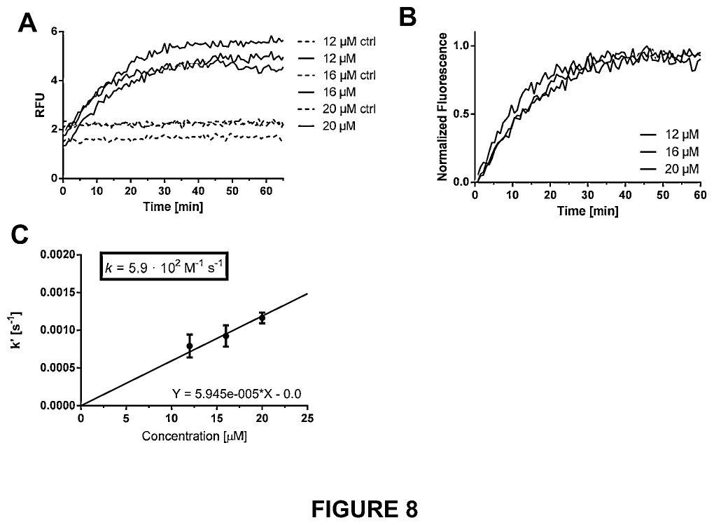

To characterize DNA-templated PINK-VdU reactions, a VdU-containing, palindromic 17-mer DNA oligonucleotide “ODN1” was synthesized (Table 1). The DMT-protected VdU phosphoramidite S4 (Scheme 2)^{[51]} was incorporated into the oligomer utilizing standard solid phase supported DNA synthesis. ODN1 was purified using reverse-phase HPLC and annealed with a complimentary strand to form a duplex. Upon mixing PINK with the VdU-containing duplex a highly rapid, fluorogenic reaction was observed. The oxidized pyridazine, analogous to compound 6 was identified as the main reaction product on the DNA by means of high-resolution mass spectrometry. By tracking the fluorescence changes under pseudo-first order conditions (FIG. 8), an apparent rate constant k=590 M⁻¹ s⁻¹ was determined for the reaction between PINK and the duplex DNA containing VdU. This value is approximately 60,000-fold higher than the non-templated reaction between PINK and VdU and is among the fastest reported bioorthogonal labeling reactions reported to date.^{[49,52]}

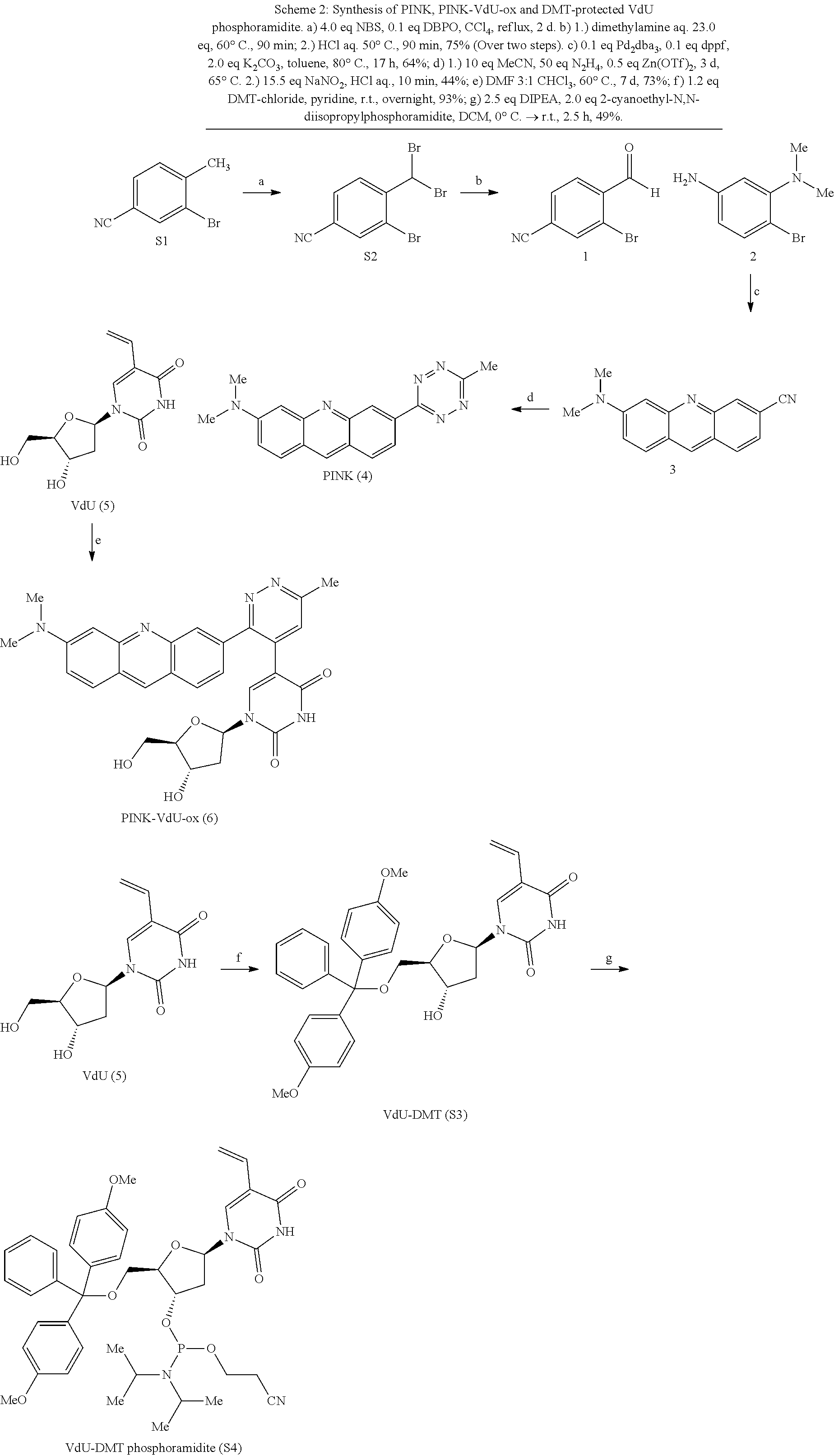

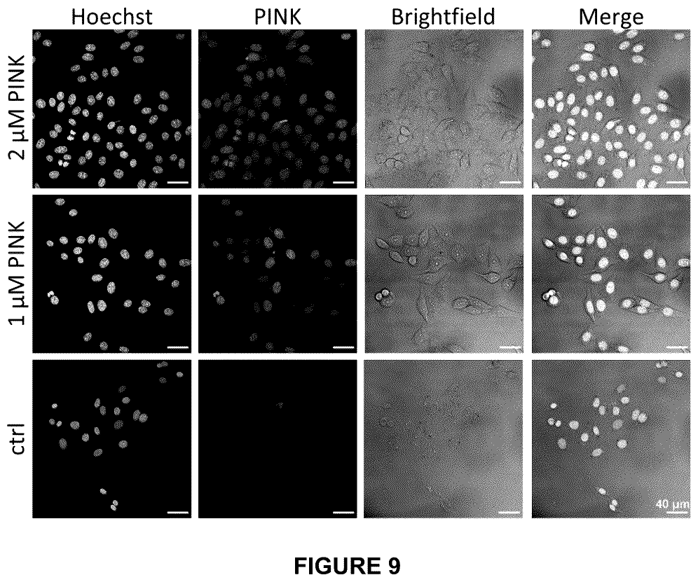

To evaluate the ability of PINK to enter live cells and undergo fluorogenic reactions with vinyl groups in native chromatin, Hela cells were grown in rich media containing 10 μM VdU for 15 h and subsequently stained with 1-2 μM PINK for 4 h. Total cellular DNA was counterstained with Hoechst 3342 (5 μM; 405 nm laser; emission 410-480 nm) for 10 minutes prior to imaging PINK (514 nm laser; emission 570-700 nm). Control (ctrl) samples were treated with PINK but not VdU. All cells were treated and maintained in DMEM media containing 10% FBS and grown in 5% CO₂ at 37° C. Live cell images were acquired using a confocal microscope. Live-cell fluorescence microscopy revealed a pattern consistent with metabolic labeling, where pairs of daughter cells with bright pink fluorescence were only observed in the VdU-treated cells, and excellent co-localization was observed between PINK and the non-covalent stain Hoechst 33342 (FIG. 9). Toxicity studies indicated that low micromolar concentrations of PINK were not toxic to cells.

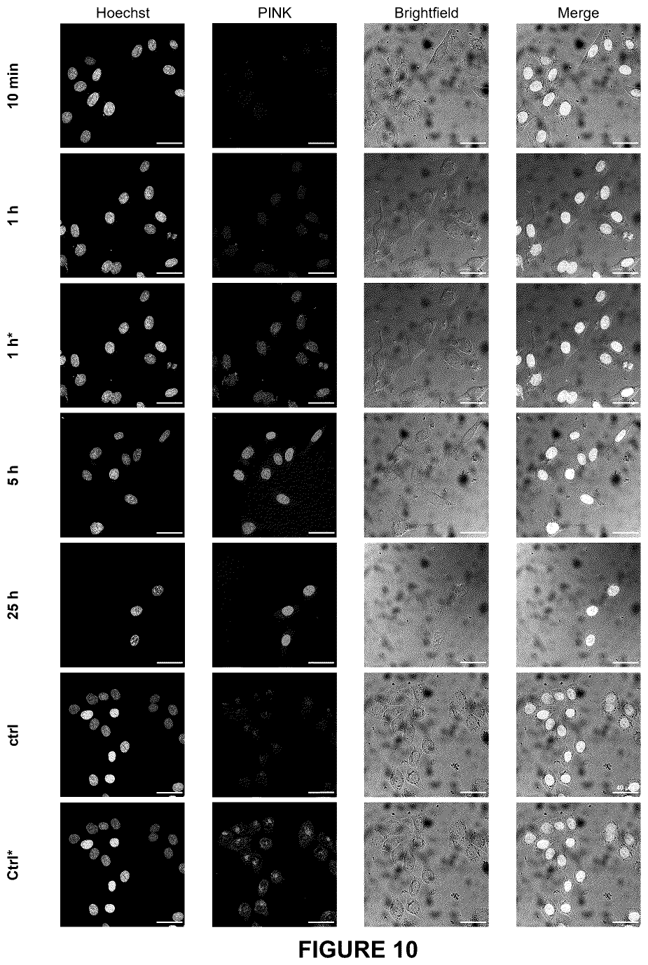

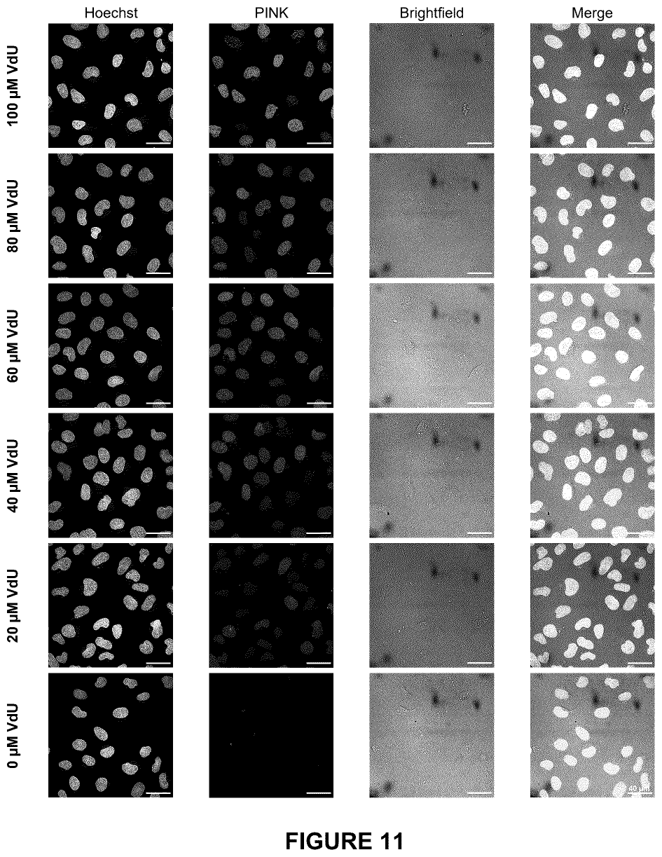

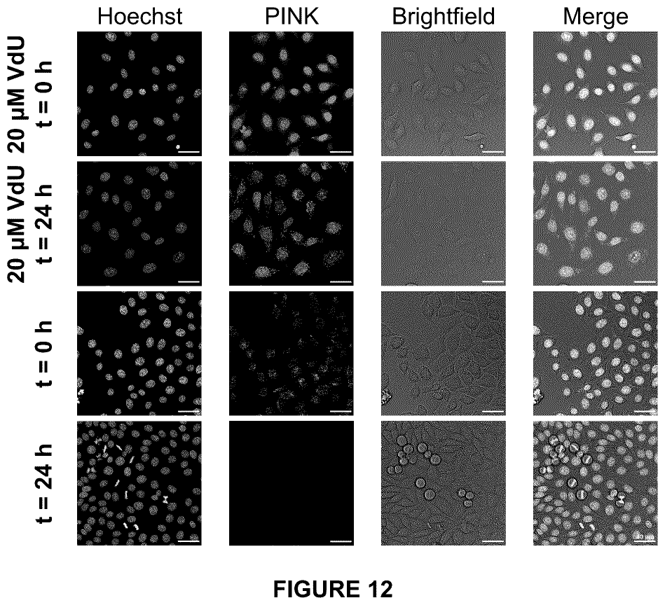

The intracellular reaction kinetics of PINK staining was next investigated. Cells were seeded at a concentration of 2.5×10⁴ cells/mL, allowed to settle overnight, and incubated with 100 μM VdU for 24 h. Cells were stained with 10 μM PINK between 10 min and 25 h, co-stained with Hoechst 33342 (5 μg/mL), and images were acquired on a confocal microscope. Samples were optimized for an ideal signal to noise ratio after staining for 1 h, illustrating a sufficiently bright signal at this stage. Ctrl samples were exposed to PINK for 25 h, thus explaining the relatively high background. It was found that a duration of 5 h to be ideal to achieve complete labeling, but a clear signal above background could already be observed after an incubation time of only 1 h (FIG. 10). Labeling was efficient in multiple cell lines. U2OS cells were seeded in μ-slide 8-well (Ibidi®) (3.0×10⁴ cells in 200 μL; 1.5×10⁵ cells/mL) in DMEM and given 20 h to settle. Cells were aspirated and VdU (0-100 μM in 200 μL DMEM) was added. Cells were incubated for 18 h, aspirated, and PINK (5 μM in 200 μL DMEM, 0.05% DMSO) was added. Cells were incubated for 5 h, aspirated, and Hoechst 33342 (2 μg/mL in 200 μL DMEM) was added for 15 min. Cells were aspirated and the media was exchanged for 200 μL DMEM (-Phenol Red). Images were acquired on a confocal microscope (FIG. 11). PINK's fluorescent signal was retained in living cells 24 h after being removed from the media. Cells (5.0×10⁴ cells/mL) were seeded and allowed to settle overnight. Cells were incubated with 20 μM VdU for 16 h and stained with 2 μM PINK for 5 h, Cells were aspirated and fresh media (w/o Phenol Red) was added. Control Samples received identical treatment, but were not exposed to VdU. Cells were co-stained with Hoechst 33342 (5 μg/mL) for 10 min right before live cell images were acquired on a confocal microscope at t=0, 24 h (FIG. 12).

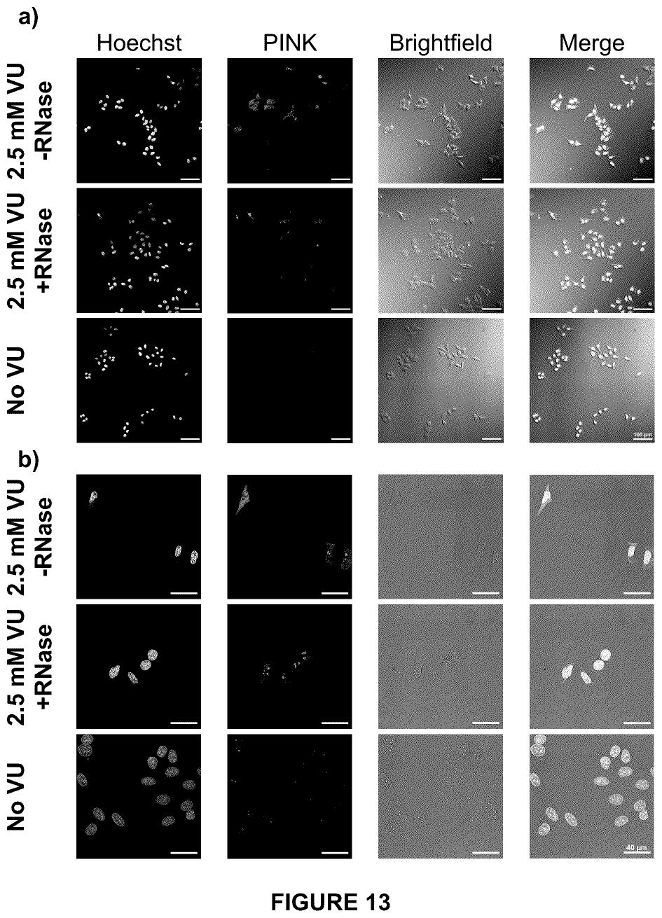

The labeling approach was extended to vinyl-modified RNA. Hela cells were seeded in ibidi® 8-well plates (5.000 cells per well) and allowed to settle overnight. Cells were then incubated with 5-vinyl uridine (VU, 2.5 mM) for 24 h. After 19 h, PINK (100 μM final concentration) was added for 5 h. cells were incubated for additional 5 h. Cells were fixed and optionally treated with RNase A/T1 mix (100 μg/mL RNase A; 250 U/mL RNase T1). Total cellular DNA was counterstained with Hoechst 33342 (5 μg/mL). Images were acquired on confocal microscopes under 20× (a) or 63× (b) magnification (FIG. 13). In accordance with successful live-cell metabolic RNA labeling, confocal microscopy revealed a strong fluorescent signal, which was strongly diminished, if the cells were treated with RNase A and T1 after fixation.

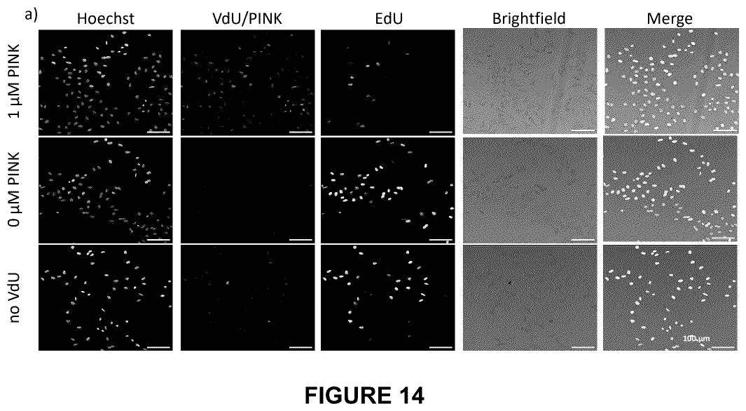

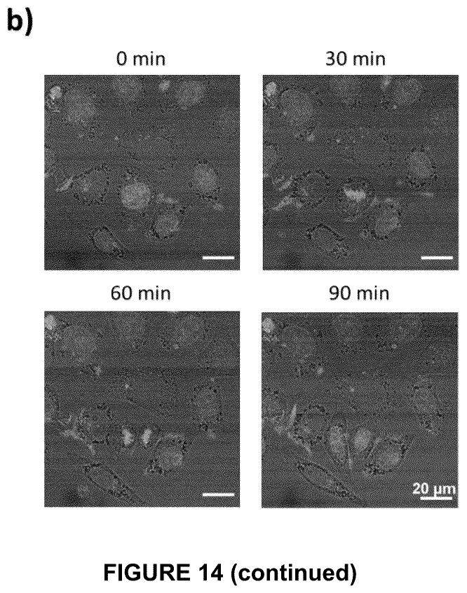

To investigate the potential impact of VdU-PINK labeling on subsequent DNA synthesis, a pulse-chase experiment was performed with 5-ethynyl-2′-deoxyuridine (EdU) (FIG. 14, a). Hela cells were grown in cell media containing 20 μM VdU for 16 h, aspirated, treated with 1 μM PINK for 4 h, aspirated and treated with 10 μM EdU for 4 h. After fixation and DNA denaturation, cells were stained with AF488-azide (488 nm laser; emission 495-540 nm), and total cellular DNA was counterstained with Hoechst 33342 (405 nm laser; emission 410-480 nm). The “No VdU” cells were incubated with 1 μM PINK but not VdU. PINK+ and EdU+ “double positive” cells were observed as having co-localized nuclear staining. These remarkable results indicated that the PINK-VdU reaction product did not cause cell cycle arrest. Therefore, VdU and PINK were used to track DNA synthesis and cellular division using live-cell, time-lapsed imaging. For optimal live-cell visualization, cells were incubated with 100 M VdU for 23 h and subsequently labeled with 1 μM PINK for 4 h prior to imaging, allowing for the recording of full mitotic cycles over 2.5 hours (FIG. 14, b). Live cell images were acquired using a confocal microscope equipped with a stage-top incubator at 37° C. and at 5% CO₂. These results provide examples of a “mix and measure” fluorogenic assay for DNA replication in living cells.

In summary, PINK was developed making use of a dual enhancement strategy where the acridine moiety of PINK serves both as a fluorescent probe as well as a rate-enhancing intercalating agent, at the same time, the tetrazine serves as the fluorescence quenching group and bioorthogonal functional group. PINK is an example of a fluorogenic intercalating agent exhibiting enhanced fluorescence upon covalently reacting with nucleic acids in living cells. Previous studies involving the chemical modification of cellular vinyl-modified DNA by bioorthogonal methods have depended on cell fixation and DNA denaturation to facilitate tetrazine-alkene reactions.^{[20,30,31,54,55]} The new capability of reacting bioorthognal functional groups in native DNA and RNA will open up new opportunities in biological and translational research.

### Example 2—Materials and Methods

All reagents were obtained in the highest available commercial grade from Sigma Aldrich™, Fisher Scientific™ or Fluorochem™, and used without additional purification. Reactions sensitive to water or air were conducted under an inert gas atmosphere (nitrogen, argon) using anhydrous solvents and standard Schlenk techniques. Analytical thin-layer chromatography was performed on pre-coated 250 μm thick silica gel 60 F254 plates and visualized by ultraviolet light. Flash column chromatography was performed using 40-63 μm silica gel and compressed air. NMR were measured with a Bruker AVIII-400 or AVIIIHD 500 (400 MHZ for ¹H, 100 MHz for ¹³C or 500 MHz for ¹H, 125 MHz for ¹³C). ¹³C spectra were recorded broadband proton decoupled. Chemical shifts (δ) are given in parts per million (ppm) and are reported relative to residual solvent peaks: CDCl₃ (δH 7.26, δC 77.0 ppm), methanol-d₄ (δH 3.31, δC 49.0 ppm). Coupling constants (J) are given in Hertz (Hz), and the following abbreviations are used to describe multiplicities: s=singlet, d=doublet, t=triplet, q=quartet, quint=quintet, sext=sextet, m=multiplet, dd=doublet-doublet, ddd=doublet-doublet-doublet, dt=doublet-triplet, dq=doublet-quartet, br=broad. Mass spectra were recorded on an Advion expression CMS, and high-resolution mass spectra were obtained on a Bruker MaXis high-resolution QTOF or a Thermo QExactive high-resolution Orbitrap. Masses are given as m/z.

Photophysical Measurements. Stock solutions (1-10 mM) of investigated compounds were prepared in DMSO and stored at −20° C. DMSO concentrations were less than 1% after dilution into the respective solvent/aqueous buffer. Measurements were collected on a SpectraMax M5 plate reader (Molecular Devices, Sunnyvale, CA, USA) in 1 cm path-length quartz cuvettes. Quantum yields were obtained by excitation at 488 nm using Rhodamine 6G ((=0.94) in EtOH (n=1.3624) as a fluorescent standard.^{[1]} Quantum yields were calculated using the equation I:

$${ϕ}_{X} = {ϕ}_{S T} ( \frac{{Grad}_{X}}{{Grad}_{S T}} ) ( \frac{{η}_{X}^{2}}{{η}_{s τ}^{2}} ) \qquad ( Equation 1 )$$

where φ_{ST} is the quantum yield of the fluorescent standard, Grad_{X} and Grad_{R} are the gradients obtained by plotting integrated emission against their respective optical densities at 488 nm. η_{X} and n_{ST} are the refractive indexes of the sample and reference solvent, respectively.

Fluorescence Turn-on Measurement. 25 μM PINK was reacted with 5 mM VdU (200 eq) in MeCN 9:1 DMSO in a 1 cm quartz cuvette at 25° C. Fluorescence spectra (excitation: 480 nm; emission: 500-750 nm) were acquired on a Horiba Duetta spectrometer over the course of 46 h.

PINK Stability Test. Fluorescence changes (excitation: 500 nm, emission: 590 nm; 515 nm cutoff filter) of 20 μM PINK were monitored in the presence of 1 mM base pairs of calf thymus (CT) DNA (50 eq) in PBS pH 7.4 (10% DMSO) followed by addition of 5-norbornene-2-methanol (2 mM, 100 eq). Measurements were conducted on a SpectraMax M5 plate reader (Molecular Devices, Sunnyvale, CA, USA) in 1 cm path-length quartz cuvettes.

Binding Affinity Measurements of PINK with CT-DNA. Binding of PINK to CT-DNA was assessed by monitoring changes of absorbance collecting measurements on a SpectraMax M5 plate reader (Molecular Devices, Sunnyvale, CA, USA) in 1 cm path-length quartz cuvettes. Small volumes of a concentrated CT-DNA solution (10 mM base pairs, 0-16 μL) were added to 1 mL of 10 μM PINK in 50 mM NaOAc aq. pH 5.2 (0.2% DMSO). Overall volume changes were 1.6% at most. Acidic conditions were required to ensure sufficient solubility of PINK. Binding of PINK to CT-DNA and determination of an apparent binding constant (K_{D}) was calculated by plotting changes of absorbance at 575 nm.

Viscosity Measurements of CT-DNA Solutions. Viscosity of DNA solutions was determined by measuring the flowtime in a Cannon-Manning semi-micro size 75 viscosimeter. Temperature regulation to room temperature was achieved by submerging the viscosimeter in a water-filled glass beaker. Separate solutions containing between 0 and 240 μM PINK with 300 μM base pairs CT-DNA in 50 mM NaOAc aq. pH 5.2 (2.4% DMSO) were freshly prepared and incubated for at least 1 h to ensure full equilibrium. Flow times for each solution were measured in triplicates, and eventual viscosity data were obtained from three independent experiments. Flow times ranged from 195.5 s to 269.1 s. Viscosity is calculated as η=t−t₀, where t describes the flow time of a respective sample, while to is the flow time of the buffer alone (135.7 s).

Oligodeoxynucleotide Synthesis and Purification. Unmodified sequences were obtained from Sigma Aldrich as HPLC-purified products. Canonical DNA phosphoramidites, solid supports and other necessary reagents were purchased from LinkTech. VdU-phosphoramidite was freshly synthesized as described below and dissolved in dry MeCN at 25 mg/ml immediately prior to use. Modified oligodeoxynucleotides were synthesized on a 1.0 μmol scale using a Bioautomation Co. Mermade 4 DNA synthesizer according to the standard-trityl-off procedure. Synthesis was monitored by DMT deprotection. Upon completion, sequences were cleaved from the solid support and deprotected by treatment with 1.0 mL of 33% aqueous ammonium hydroxide at 55° C. overnight in a 1.5 mL screw-cap tube. The resulting solutions were filtered through Whatman FP30/0.2 CA-S 0.2 μm syringe filters, which were then washed with deionized water. Obtained solutions were lyophilized to dryness. Final purification was performed by HPLC column chromatography on an analytical C-18 reverse-phase column (Waters xBridge® C18 3.5 μm 4.6×150 mm column) using a Varian 140 Pro Star HPLC system. The following gradient of MeCN: 0.1 M aqueous triethylammonium acetate pH 7.4 (TEAA) was applied: 5:95 to 23:77 over 30 min with a flow rate of 0.4 mL/min. Elution was monitored by UV absorption at 260 nm. Peaks from several runs were collected and lyophilized to dryness three consecutive times after addition of fresh deionized water in total to fully remove trace amounts of triethylamine and acetic acid.

Oligodeoxynucleotides were analyzed by LC-MS using a Dionex Ultimate 3000 UHPLC coupled to a Bruker Maxis Impact QTOF in negative ESI mode. Samples were run through a Phenomenex Luna C18(2)-HST column (2.5 μM 120A 2.1×100 mm) using a gradient of 90% mobile phase A (100 mM HFIP, 5 mM NEt₃ in H₂O) and 10% mobile phase B (MeOH) to 40% mobile phase A and 60% mobile phase B over 20 minutes. The data was processed, and spectra were deconvoluted using the Bruker DataAnalysis software version 4.2.

Oligodeoxynucleotide stock solutions were prepared in deionized (milli-Q) water and their concentrations were determined by absorbance at 260 nm using the molar extinction coefficient calculated using a nearest-neighbor model.^{[2]} Duplex DNA was prepared by mixing 1 eq of modified DNA (flank_G) with 1.1 eq of the complimentary sequence (compl_G) in 10 mM NaH₂PO₄ pH 7. The solution was heated to 95° C. for 5 min and slowly cooled to r.t. overnight.

Preparation of PINK-Modified Oligodeoxynucleotide “Flank_G_PINK”. Vinyl-modified oligodeoxynucleotide (ODN) duplexes were prepared as described above. 10 μM of vinyl-DNA was reacted with 100 μM PINK in 50 mM NaOAc buffer pH 5.2 (2% DMSO) overnight at 37° C. The modified DNA was purified by using Nap™-5 columns (Cat-#: 17-0853-01) eluting with ddH₂O. DNA containing fractions were combined based on A260 absorbance, evaporated, and taken up in 10 mM NaH₂PO₄ pH 7. Samples were then reannealed as described above. Concentration was quantified according to the A260 value assuming equal molar extinction coefficients for PINK- and vinyl-modified ODN.

Kinetic Measurements with the VdU Nucleoside. Second order reaction rates between PINK and VdU were determined under pseudo first order conditions with a large excess of the dienophile over the tetrazine. The increase of emission at 590 nm (excitation: 500 nm, 515 nm cut-off filter) was used to monitor the progress of the reaction. Measurements were performed on a SpectraMax M5 plate reader (Molecular Devices, Sunnyvale, CA, USA) in 384 well plates (Greiner bio-one, cat-#871906; 50 μL per well; covered with 20 μL spectroscopic grade silicon oil to avoid evaporation). Solutions contained final concentrations of 100 μM PINK with 1-5 mM VdU (10-50 eq) in 50 mM aq. NaOAc buffer pH 5.2 (27% DMSO). The acidic concentrations and high DMSO content were necessary to ensure sufficient solubility of PINK and VdU respectively. Samples were measured over 18 h in intervals of 10 min. Control conditions (0 mM VdU) indicate full stability and negligible background signal of PINK under these conditions. Reactions rates were calculated using pseudo first order approximations fitting the measurements to mono exponential equations. The reported reaction rate is an arithmetic mean determined from three independent experiments.

Kinetic Measurements with ODNs. Second-order rate constants for the reaction between vinyl-modified ODN and PINK were determined under pseudo first order conditions employing an excess of the tetrazine. The increase in emission at 590 nm (excitation 530 nm; 550 nm cut-off filter) was used to monitor the progress of the reaction. Measurements were performed on a SpectraMax M5 plate reader (Molecular Devices, Sunnyvale, CA, USA) in 384 well plates (Greiner bio-one, cat-#871906; 40 μL per well). Solutions contained a final solution of 4 μM vinyl-modified ODN (flank_G) with 12-20 M of PINK (3-5 eq) in 50 mM aq. NaOAc buffer pH 5.2 (0.8% DMSO). The acidic concentrations were necessary to ensure sufficient solubility of PINK. Samples were measured over the course of 1 h in intervals of 45 s. Control Conditions (12-20 μM PINK without vinyl-modified ODN) indicate full stability of PINK and negligible background signal under these conditions. Reactions rates were calculated using pseudo first order approximations fitting the measurements to mono exponential equations. The reported reaction rate is an arithmetic mean determined from three independent experiments.

Eukaryotic Cell Culture. Eukaryotic cells (HeLa, U2OS) were cultivated at 37° C., 5% CO₂ in DMEM (Gibco) containing 4.5 g/l glucose, 10% FBS (Gibco), 50′000 units Penicillin, and 50 mg Streptomycin per L (Sigma Aldrich), and 1% MEM non-essential amino acids (Sigma Aldrich) Cells were grown to confluency and passaged every 2 to 4 days using a Trypsin-EDTA solution (Sigma Aldrich). Cells were counted using an Olympus Automated Cell Counter Model R1 for the determination of seeding density.

Resazurin Assay. HeLa or U2OS cells were seeded in 96-well plates at a density of 7,500 cells per well and incubated overnight. The supernatant was removed and 100 μL fresh media containing PINK (0-12.5 μM; 0.1% DMSO) was added. Cells were grown for 24 or 72 h, at which point 10 μL resazurin (880 μM in PBS; final concentration: 80 μM) was added. After incubation for 3 h, the fluorescence emission at 590 nm (excitation 560 nm; cut-off: 575 nm) was measured using a SpectraMax M5 plate reader (Molecular Devices, Sunnyvale, CA, USA). LC₅₀ values were determined from two independent replicates.

Confocal Laser Scanning Microscopy (CLSM). Confocal Laser Scanning Microscopy (CLSM) was performed on a) a CLSM Leica SP5 Mid UV-VIS (Leica Microsystems) equipped with a HC PL APO Leica 10× air objective (NA 0.4, WD 2.2), HC PL APO Leica 20× multi immersion objective (NA 0.7, WD 0.25), HCX PL APO Leica 40× oil immersion objective (NA 1.25, WD 0.1), HCX PL APO Leica 63× oil immersion objective (NA 1.4, WD 0.1), or HCX PL APO 37° C. Leica 63× glycerol objective (NA 1.3, WD 0.28); b) CLSM Leica SP8 HC PL FLUOTAR 10× air objective, HC PL APO CS2 20× immersion objective, HC PL APO CS2 63× oil objective, or a HC PL APO 37° C. CS2 63× glycerol objective; c) Leica Stellaris 5 LIAchroic (Leica Microsystems) equipped with a HC PL APO 20× multi immersion objective (0.75 IMM CORR CS2), or a HC PL APO 63×/1.40 OIL CS2 (FWD: 0.14 mm); or d) Nikon Ti2 inverted spinning disk (CREST X-Light V2 L-FOV) confocal setup equipped with a Nikon PlanApo 20× 0.75NA air objective. DAPI and Hoechst 33342 were excited at 405 nm, and emission was sampled between 410 and 480 nm; Alexa FL488, was excited at 488 nm and emission was sampled between 495 and 540 nm; PINK was excited at 512, 546, 552 or 561 nm, and emission was recorded between 530 and 700 nm (for 512 nm excitation), or 575 nm and 700 nm (for 546 nm excitation) respectively. HyD detectors were used. Z-stacks were recorded with a step size between 0.1 μm and 10 μm. Image analysis was performed using Leica LAS AF Lite 2.6.3 (Leica Microsystems) and Fiji 1.50i (Wayne Rasband, National Institutes of Health, USA).

Live-Cell Metabolic Labeling. Cells were seeded in μ-slide 8-well chambers (ibidi®), cat-#: 80826) at densities of 2.5×10⁴-1.5×10⁵ cells per mL and allowed to settle overnight. They were aspirated and incubated with variable concentrations of VdU. After incubating for varying times, cells were aspirated, and incubated with variable concentrations of PINK for 4-5 h in phenol red-free media. Right before imaging, total cellular DNA was counterstained with Hoechst 33342 (5 μg/mL) for 10 min. Before addition, all compounds were freshly diluted into DMEM from DMSO-stock solutions, making sure that the final DMSO concentration would not exceed 0.1%.

Metabolic RNA Labeling. Hela cells were seeded in μ-slide 8-well chambers (ibidi®), cat-#: 80826) at a density of 2.0×10⁴ cells per mL (5.000 cells per well) and allowed to settle overnight. Cells were aspirated and 5-vinyl uridine (VU, 2.5 mM, 0.1% DMSO) was added. Cells were incubated for 19 h, at which point PINK (100 μM; 0.2% DMSO final concentrations) was added. After additional 5 h, cells were aspirated, washed with PBS, and fixed with 3.7% PFA in PBS for 15 min. Cells were quenched with 50 mM glycine/NH₄Cl for 5 min, washed with PBS, and permeabilized twice with 0.2% Triton X-100 in PBS for 5 min each followed by washing with PBS, twice 100 mM NaOAc buffer (pH 5.2), 10% DMSO in PBS, and PBS for 5 min each. For RNA digestion, cells were incubated with RNase A/T1 mix (Thermo Scientific™ EN0551; 100 μg/mL RNase A; 250 U/mL RNase T1) for 1 h in RNase buffer (10 mM Tris-HCl, 300 mM NaCl, 5 mM EDTA, pH 7.6).Cells were subsequently washed twice with PBS for 5 min. Total cellular DNA was counterstained using Hoechst 33342 (5 μg/mL) for 15 min, followed by washes with PBS, 0.2% Triton X-100 in PBS, and PBS for 5 min each.

Pulse-Chase Labeling. In each well of a 24-well plate, one 10 mm diameter cover slide was placed. Hela cells were seeded at 1.2×10⁵ cells per mL and allowed to settle overnight (22 h). Pulse: Cells were aspirated and incubated with VdU (20 μM) for 16 h, aspirated again and incubated with PINK (0, 1, 10 μM for 4 h). Chase: Cells were aspirated and treated with EdU (0, 10 μM) in DMEM for 4 h. Cells were fixed with 3.7% paraformaldehyde (PFA) for 15 min, quenched with 50 mM glycine/NH₄Cl for 5 min, and washed with PBS for 5 min. Cells were permeabilized with 0.2% Triton X-100 in PBS for 5 min at r.t., 15 min on ice, and washed three times with PBS. DNA was denatured in 2M HCl aq. for 30 min, followed by washing with PBS for 5 min, neutralization with 0.1M Na₂B₄O₇·10H₂O for 10 min, and washing three times with PBS for 5 min. Click staining was performed for 1 h in the dark by incubating coverslips upside-down on 25 μL solutions with final concentrations of 10 μM AF488 azide (Invitrogen, cat-#: A10266), 1 mM CuSO₄, 2 mM THPTA, and 10 mM sodium L-ascorbate in PBS. Coverslips were washed twice with PBS for 5 min, and the total cellular DNA was counterstained with DAPI (5 μM in PBS) for 15 min, washed twice with PBS and with H₂O for 5 min each. Coverslips were glued upside down onto microscopy slides using Glycergel (11 μL, Dako) and dried overnight. Images were acquired on a confocal microscope. DAPI was excited with a 405 nm laser sampling emission between 410-480 nm, AF488 with a 488 nm laser sampling emission between 495-540 nm, and PINK with a 561 nm laser sampling emission between 600-700 nm.

Live Cell Time Lapse Imaging. Cells were seeded in μ-slide 8-well chambers (ibidi®), cat-#: 80826) at a density of 2.0×10⁵ cells per mL and allowed to settle overnight. Cells were aspirated and incubated with 100 μM VdU for 23 h. Cells were aspirated and incubated with 1 μM PINK for 5 h. Cells were aspirated, and fresh DMEM (-Phenol Red) was added. Images were acquired on a confocal microscope with an interval of 30 min between each frame. PINK was excited with a 514 nm laser and emission was sampled between 525-650 nm.

### Example 3—Synthesis Detail

### 3-Bromo-4-(dibromomethyl)benzonitrile (S2)

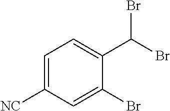 [SMILES: N#Cc1ccc(C(Br)Br)c(Br)c1]

3-Bromo-4-(dibromomethyl)benzonitrile was prepared according to a modified literature procedure.^{[3]} 3-Bromo-4-methylbenzonitrile (S1, 1.00 g, 5.10 mmol, 1.0 eq) was dissolved in CCl₄, and NBS (3.63 g, 20.4 mmol, 4.0 eq) and DBPO (124 mg, 510 μmol, 0.1 eq) was added. The reaction mixture was refluxed for 2 d and filtered. The solid was washed with DCM, and the filtrate was concentrated under reduced pressure to yield the crude product, which was directly taken to the next step. Analytical data in accordance with literature values.^{[4]}

3-Bromo-4-formylbenzonitrile (1)

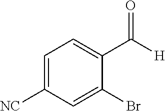 [SMILES: N#Cc1ccc(C=O)c(Br)c1]

3-Bromo-4-formylbenzonitrile was prepared according to a modified literature procedure.^{[5]} Crude 3-bromo-4-(dibromomethyl)benzonitrile (S2, 1.80 g, 5.10 mmol, 1.0 eq) was suspended in dimethylamine (40% aq., 15 mL, 13.2 g, 23.0 eq), and the reaction mixture was stirred at 60° C. for 90 min. The reaction mixture was acidified with HCl (6 M, aq.) and stirred at 50° C. for an additional 90 min. The aqueous phase was extracted with EtOAc (4×8 mL). The combined organic phases were washed with brine (8 mL), dried over MgSO₄, filtered, and concentrated under reduced pressure. The crude product was purified by flash column chromatography (10% EtOAc in hexanes) to yield 3-bromo-4-formylbenzonitrile (806 mg, 3.84 mmol, 75% over two steps) as a yellow solid. Analytical data in accordance with literature values.^{[6]}

3-Cyano-6-(dimethylamino) acridine (3)

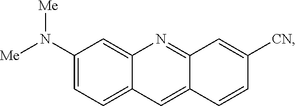 [SMILES: CN(C)c1ccc2cc3ccc(C#N)cc3nc2c1]

A flask containing N,N-Dimethyl, 1,3-phenylenediamine^{[7]} (2, 50.0 mg, 367 μmol, 1.0 eq), Pd₂(dba)₃ (8.40 mg, 9.18 μmol, 0.025 eq), dppf (10.2 mg, 18.4 μmol, 0.05 eq) and K₂CO₃ (102 mg, 794 μmol, 2.0 eq) was evacuated for 5 min and flushed with argon. 1.5 mL toluene and 3-bromo-4-formylbenzonitrile (1, 92.5 mg, 441 μmol, 1.2 eq) were added and the solution was heated to 80° C. for 17 h. Roughly 1.5 mL silica gel (SiO₂, Silicycle) were added, and the reaction mixture was concentrated under reduced pressure. Purification by flash column chromatography (20→50% EtOAc in hexanes) gave 3-cyano-6-(dimethylamino) acridine as a red solid (57.7 mg, 233 μmol, 64%).

¹H NMR (500 MHZ, CDCl₃) δ [ppm]=8.47 (s, 1H), 8.38 (s, 1H), 7.89 (d, J=8.5 Hz, 1H), 7.78 (d, J=9.4 Hz, 1H), 7.40 (d, J=8.5 Hz, 1H), 7.32 (d, J=9.4 Hz, 1H), 7.05 (s, 1H), 3.18 (s, 6H).

¹³C NMR (125 MHZ, CDCl₃) δ [ppm]=152.3, 152.2, 147.9, 135.5, 134.8, 129.9, 129.2, 125.8, 122.9, 122.4, 119.4, 119.3, 113.0, 103.4, 40.5.

ESI-HRMS: calculated for C₁₆H₁₄N₃⁺ [M-H]⁺: 248.1182; found: 248.1176.

PINK (5)

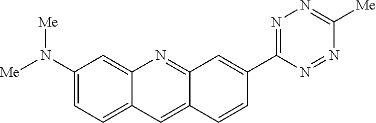 [SMILES: Cc1nnc(-c2ccc3cc4ccc(N(C)C)cc4nc3c2)nn1]

3-Cyano-6-(dimethylamino)-acridine (3, 56.7 mg, 229 μmol, 1.0 eq) was dissolved in hydrazine (anhydrous, 1 M in THF, 11.5 mL, 11.5 mmol, 50 eq), and MeCN (312 μL, 2.29 mmol, 10.0 eq) and Zn(OTf)2 (41.7 mg, 115 μmol, 0.5 eq) were added. The reaction mixture was heated to 65° C. for 3 days. NaNO2 (245 mg, 3.55 mmol, 15.5 eq) was added and the mixture was acidified to pH 3 with HCl (6M aq., 2 mL). (Caution! This step generates highly toxic nitrous gases). 60 mL NaHCO₃(aq. sat.) were added, and the aqueous phase was extracted with DCM (4×15 mL). The combined organic phases were washed with brine (30 mL), dried over MgSO₄ and concentrated under reduced pressure. Purification by flash column chromatography (0→1% MeOH in DCM) yielded PINK (4, 32.0 mg, 101 μmol, 44%) as a dark red solid.

¹H NMR (500 MHZ, CDCl₃) δ [ppm]=9.41 (s, 1H), 8.58 (s, 1H), 8.49 (dd, J=8.8, 1.7 Hz, 1H), 8.08 (d, J=8.7 Hz, 1H), 7.85 (d, J=9.3 Hz, 1H), 7.34 (dd, J=9.3, 2.5 Hz, 1H), 7.18 (d, J=2.4 Hz, 1H), 3.20 (s, 6H), 3.13 (s, 3H).

¹³C NMR (125 MHZ, CDCl₃) δ [ppm]=167.3, 164.6, 152.1, 152.0, 149.3, 135.4, 133.0, 129.7, 129.6, 129.2, 126.5, 122.3, 121.1, 119.0, 104.0, 40.5, 21.3.

ESI-HRMS: calculated for C₁₈H₁₇N₆⁺ [M-H]⁺: 317.1509; found: 317.1508.

VdU: 5-vinyl-2′-deoxyuridine (5)

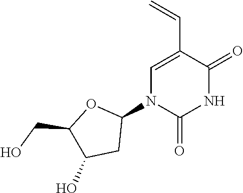 [SMILES: C=Cc1cn([C@H]2C[C@H](O)[C@@H](CO)O2)c(=O)[nH]c1=O]

A solution of 5-iodo-2′-deoxyuridine (IdU, 2.00 g, 5.65 mmol, 1.0 eq), potassium trifluoro vinyl borate (vinyl-BF₃K, 1.13 g, 8.47 mmol, 1.5 eq) and K₂CO₃ (1.56 g, 11.9 mmol, 2.0 eq) in MeOH (75 mL) were sparged with argon for 1 h. Pd(OAc)₂ (127 mg, 565 μmol, 0.1 eq) and triphenylphosphine (PPh₃, 296 mg, 1.13 mmol, 0.2 eq) were added, and the reaction mixture was heated to 70° C. for 21 h. The reaction mixture was concentrated, and VdU was purified by three consecutive turns of flash column chromatography (0-8% MeOH in DCM). It was obtained as a white solid (971 mg, 3.82 mmol, 68%). Analytical data in accordance with literature values.^{[8]}

### PINK-VdU-ox (6):

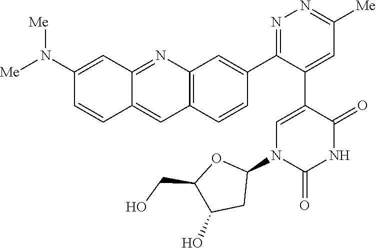 [SMILES: Cc1cc(-c2cn([C@H]3C[C@H](O)[C@@H](CO)O3)c(=O)[nH]c2=O)c(-c2ccc3cc4ccc(N(C)C)cc4nc3c2)nn1]

VdU (5, 12.2 mg, 48.0 μmol, 1.0 eq) and PINK (4, 30.2 mg, 95.5 μmol, 2.0 eq) were dissolved in 2 mL (DMF 3:1 CHCl₃) and the reaction mixture was heated to 60° C. for 7 days. The reaction mixture was concentrated under reduced pressure, and purification by flash column chromatography (0-15% MeOH in DCM) yielded PINK-VdU-ox (18.9 mg, 35.0 μmol, 73%) as a red solid.

¹H NMR (500 MHZ, methanol-d₄) δ [ppm]=8.72 (s, 1H), 8.26 (s, 1H), 8.05 (s, 1H), 8.02 (d, J=8.6 Hz, 1H), 7.86 (d, J=9.5 Hz, 1H), 7.74 (s, 1H), 7.63 (dd, J=8.6, 1.7 Hz, 1H), 7.38 (dd, J=9.5, 2.5 Hz, 1H), 6.80 (s, 1H), 6.19 (t, J=6.4 Hz, 1H), 4.15 (dt, J=6.8, 3.7 Hz, 1H), 3.80 (q, J=3.3 Hz, 1H), 3.59 (dd, J=13.5, 3.1 Hz, 1H), 3.19 (s, 6H), 2.79 (s, 3H), 2.13 (ddd, J=13.6, 6.2, 3.8 Hz, 1H), 1.88 (dt, J=13.3, 6.5 Hz, 1H).

¹³C NMR (125 MHZ, methanol-d₄) δ [ppm]=163.1, 159.6, 154.4, 151.6, 151.3, 148.2, 144.0, 141.2, 138.6, 134.2, 131.2, 130.9, 130.3, 126.8, 125.7, 123.3, 119.7, 111.4, 101.3, 89.0, 86.7, 71.3, 62.3, 41.7, 40.5, 21.1.

ESI-HRMS: calculated for C₂₉H₂₉N₆O₅⁺[M-H]⁺: 541.2194; found: 541.2179.

DMT-VdU (S3): 5′-O-(4,4-dimethoxytrityl)-5-vinyl-2′-deoxyuridine

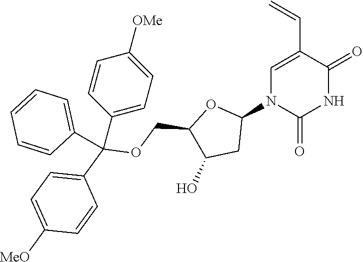 [SMILES: C=Cc1cn([C@H]2C[C@H](O)[C@@H](COC(c3ccccc3)(c3ccc(OC)cc3)c3ccc(OC)cc3)O2)c(=O)[nH]c1=O]

5′-O-(4,4-dimethoxytrityl)-5-vinyl-2′-deoxyuridine was prepared according to a modified literature procedure. [9] 5-Vinyl-2′-deoxyuridine (5, 87.0 mg, 342 μmol, 1.0 eq) was dissolved in pyridine (2 mL), and DMT-chloride (139 mg, 411 μmol, 1.2 eq) was added. The reaction mixture was stirred overnight and concentrated under reduced pressure. Purification by flash column chromatography (30-100% EtOAc in hexanes, 1% NEt₃) afforded 5′-O-(4,4-dimethoxytrityl)-5-vinyl-2′-deoxyuridine (177 mg, 318 μmol, 93%) as an off-white solid. Analytical data in accordance with the literature.^{[9]}

DMT-VdU phosphoramidite (S4): 5′-O-(4,4-dimethoxytrityl)-3′-O-[2-cyanoethxy-(N,N-diisopropylamino)-phosphino]-5-vinyl-2′-deoxyuridine

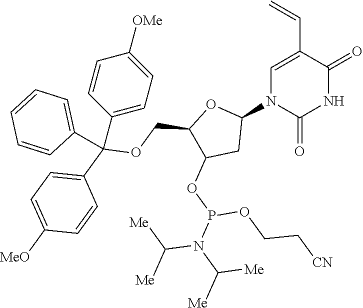 [SMILES: C=Cc1cn([C@H]2CC(OP(OCCC#N)N(C(C)C)C(C)C)[C@@H](COC(c3ccccc3)(c3ccc(OC)cc3)c3ccc(OC)cc3)O2)c(=O)[nH]c1=O]

5′-O-(4,4-dimethoxytrityl)-5-vinyl-2′-deoxyuridine was prepared according to a modified literature procedure.^{[9]} Freshly distilled DIPEA (63 μL, 46.4 mg, 359 μmol, 2.5 eq) was added to a solution of DMT-VdU (S3, 80.0 mg, 144 μmol, 1.0 eq) in DCM (1 mL), and the solution was cooled to 0° C. 2-Cyanoethyl-N,N-diisopropylchlorophosphoramidite (63 μL, 68.0 mg, 287 μmol, 2.0 eq) was added, and the reaction mixture was slowly warmed to r.t. over the course of 2.5 h. The reaction mixture was loaded straight onto a silica column and purified by flash column chromatography (60% EtOAc in hexanes; 0.5% NEt₃) to afford 5′-O-(4,4-dimethoxytrityl)-5-vinyl-2′-deoxyuridine (53.0 mg, 70.0 μmol, 49%) as an off-white solid. Analytical data in accordance with the literature.^{[9]}

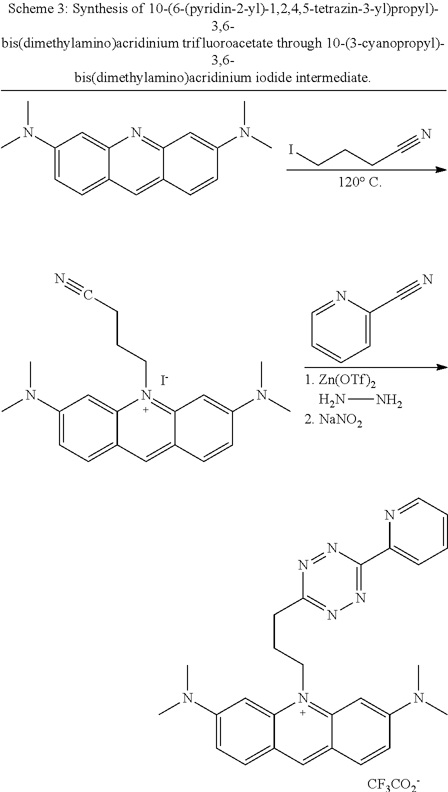

10-(3-cyanopropyl)-3,6-bis(dimethylamino) acridinium iodide (AO-495-nitrile)

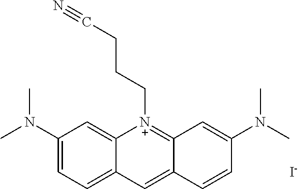

Method 1: A 25 mL sealable tube containing 63.5 mg acridine orange (239 μmol) and a stir bar was evacuated under vacuum and refilled with argon for three times. Afterwards, 3.0 mL toluene and 52 μL 4-iodobutanenitrile (487 μmol) was added to the tube. The suspension was refluxed for 24 h at 120° C. and reaction progress was monitored by crude NMR. After the reaction, the mixture was evaporated under reduced pressure to dryness and washed by 3×8 mL DCM (to the supernatant color no longer change). The precipitate was dried under vacuum to yield the product as orange to red powder (39.9 mg, 86.7 μmol, 36%).

Method 2: A 0.5-2 mL microwave reaction tube containing 59.7 mg acridine orange (225 μmol), 1 mL acetonitrile, 50 μL 4-iodobutanenitrile (469 μmol) and a stir bar was mixed under microwave at 150° C. for 3 h. After reaction, the mixture was evaporated to dryness under reduced pressure and washed by 3×8 mL DCM. The precipitate was dried under vacuum to yield the product as dark red powder (47.6 mg, 103 μmol, 46%)

¹H NMR (500 MHZ, DMSO-d₆): δ [ppm]=8.84 (s, 1H), 7.97 (d, J=9.3 Hz, 2H), 7.31 (dd, J=9.3, 2.1 Hz, 2H), 6.68 (d, J=2.2 Hz, 2H), 4.81 (t, J=8.3 Hz, 2H), 3.30 (s, 12H), 2.89 (t, J=6.8 Hz, 2H), 2.21 (p, J=7.0 Hz, 2H)

¹³C NMR (125 MHZ, DMSO) δ [ppm]=156.1, 143.7, 142.8, 133.7, 120.9, 117.0, 114.9, 92.7, 45.8, 40.9, 21.8, 14.6.

HRMS (ESI): calculated m/z for C₂₁H₂₅N₄⁺[M]⁺: 333.2074 found: 333.2073 calculated m/z for I⁻: 126.9050 found: 126.9037.

(10-(6-(pyridin-2-yl)-1,2,4,5-tetrazin-3-yl)propyl)-3,6-bis(dimethylamino)acridinium trifluoroacetate) (“AO-495-TET-py”)

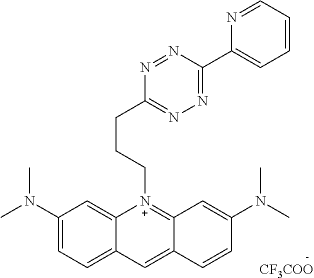

A 5 mL reaction vial containing 20.0 mg AO-495-nitrile (43.4 μmol), 8.1 mg zinc triflate (22 μmol), 46.0 mg 2-pyridinecarbonitrile (442 μmol) and a stir bar was evacuated under vacuum and refilled with argon for three times. Afterwards, 80 μL anhydrous hydrazine (2.6 mmol) was added to the tube with syringe. The mixture was sealed and stirred at 60° C. overnight and totally dried up. Another 80 μL anhydrous hydrazine was added to the tube and stirred at 60° C. for 24 h in total. The resulting mixture was dissolved with 2 mL DMSO and transferred to an Erlenmeyer flask with 15 mL of 0.5 M sodium nitrite. 5 M acetic acid in water was slowly added to the flask while stirring until no more bubbles were produced and the pH=3 (Caution! Toxic nitrogen oxide gases are produced in this step, only perform this step in a well ventilated fumehood). Potassium bicarbonate was added to adjust the pH to >7. The mixture was extracted with 20 mL DCM three times and evaporated under reduced pressure. The resulting solid was dissolved in 20% acetonitrile in water, filtered and purified by HPLC (32-40% MeCN, 0.1% TFA in water). The product was lyophilized from the fractions and obtained as an orange-red solid (2.2 mg, 3.8 μmol, 9%).

¹H NMR (500 MHZ, CD₃CN) δ [ppm]=8.94 (d, J=4.9 Hz, 1H), 8.60-8.50 (m, 2H), 8.11 (td, J=7.8, 1.7 Hz, 1H), 7.85 (d, J=9.3 Hz, 2H), 7.68 (ddd, J=7.6, 4.7, 1.1 Hz, 1H), 7.21 (dd, J=9.3, 2.1 Hz, 2H), 6.81 (d, J=2.2 Hz, 2H), 4.81 (t, J=8.5 Hz, 2H), 3.82 (t, J=6.6 Hz, 2H), 3.34 (s, 11H), 2.75 (p, J=6.6 Hz, 2H).

¹³C NMR (125 MHZ, CD₃CN) δ [ppm]=164.1, 156.0, 150.7, 143.1, 142.9, 137.7, 133.0, 126.5, 124.0, 117.0, 114.4, 92.5, 46.8, 40.3, 31.3, 21.6.

HRMS (ESI) Calculated m/z for C₂₇H₂₉N₈⁺ [M⁺]: 465.2510 found: 465.2501

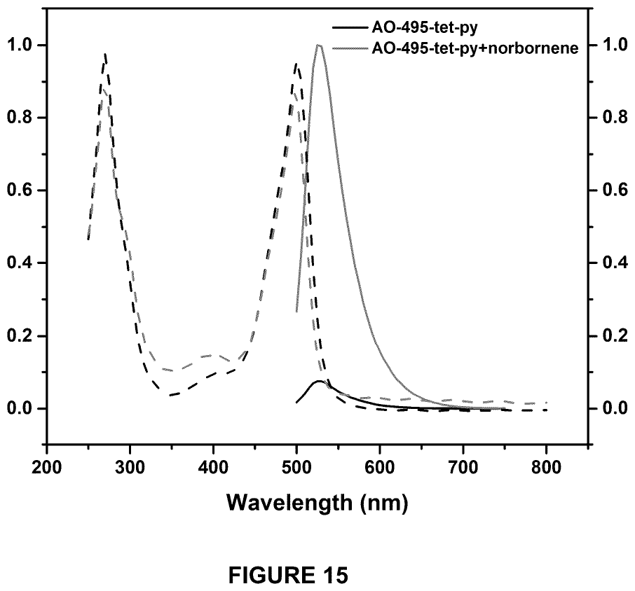

The absorbance and fluorescence intensity (excitation at 488 nm) of AO-495-TET-py in different solvents (water, PBS and acetonitrile) was measured in a quartz cuvette (light path 1 cm) using a SpectraMax M5 plate reader (Molecular Devices, Sunnyvale, CA, USA). For AO-495-tet-py, samples for photophysical characterization were prepared directly from stock solution (10 mM in DMSO) by gradient dilution. For reaction of AO-495-tet-py with 5-norbornene-2 methanol, 20 μL AO-495-tet-py stock solution (in DMSO) was mixed with 2 μL 5-norbornene-2 methanol (80× excess) in room temperature for 24 h to ensure full conversion. 3 μL of the reaction mixture was diluted in to the indicated solvent to make a 8 mM solution for following photophysical characterization. Rhodamine 6G in ethanol was used as fluorescence standard (φ=0.94). See Table 2 and FIG. 15.

TABLE 2
Obtained photophysical properties of AO-495-tet-py
before and after reaction with 5-norbornene-2 methanol.

| | Solvent | λₘₐₓ(Abs) | λₘₐₓ(Fluor.) | φ |
|---|---|---|---|---|
| AO-495-tet-py | PBS | 500 nm | 525 nm | 0.0148 |
| | Water | 500 nm | 525 nm | 0.0063 |
| | MeCN | 498 nm | 525 nm | 0.0075 |
| AO-495-tet- | PBS | 498 nm | 525 nm | 0.152 |
| py + 5- | Water | 498 nm | 525 nm | 0.168 |
| Norbornene- | MeCN | 498 nm | 525 nm | 0.240 |
| 2-methanol | | | | |

N,N-dimethyl-6-(6-(pyridine-2-yl)-1,2,4,5-tetrazine-3-yl) acridine-3-amine (Pyridyl-PINK)

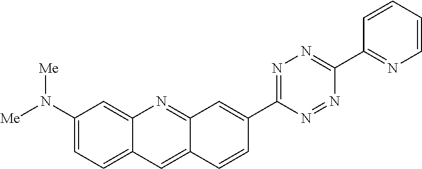 [SMILES: CN(C)c1ccc2cc3ccc(-c4nnc(-c5ccccn5)nn4)cc3nc2c1]

A tube containing 3-Cyano-6-(dimethylamino)-acridine (15.0 mg, 60.7 μmol, 1.0 eq), 2-cyanopyridine (63.2 mg, 58.4 μL, 607 μmol, 10 eq) and zinc (II) triflate (11.0 mg, 30.3 μmol, 0.5 eq) was evacuated for 5 min and refilled with argon. Hydrazine (1M in THF, 3.03 mL, 3.03 mmol, 50 eq) was added, the tube was sealed, and the reaction mixture was heated to 60° C. for 5 days. The reaction mixture was concentrated under reduced pressure and redissolved in 10 mL H₂O 1:1 MeOH. Sodium nitrite (64.9 mg, 940 μmol, 15.5 eq) was added, and the reaction mixture was acidified to pH 3 by addition of HCl (6M aq.). The solution was neutralized to pH 9 by addition of NaHCO₃. The aqueous phase was extracted with DCM (3×20 mL), and the combined organic phases were washed with brine (20 mL), dried over MgSO₄ and concentrated under reduced pressure. Purification by flash column chromatography (0-2% MeOH in DCM) yielded pyridyl-PINK (8.8 mg, 23.2 μmol, 38%) as a dark purple solid.

¹H NMR (500 MHZ, CDCl₃) δ [ppm]=9.53 (s, 1H), 8.99 (d, J=3.8 Hz, 1H), 8.75 (d, J=7.8 Hz, 1H), 8.62-8.56 (m, 2H), 8.11 (d, J=8.7 Hz, 1H), 8.04-7.98 (m, 1H), 7.85 (d, J=9.2 Hz, 1H), 7.57 (ddd, J=7.6, 4.7, 1.2 Hz, 1H), 7.34 (dd, J=9.4, 2.4 Hz, 1H), 7.19 (d, J=2.4 Hz, 1H).

ESI-HRMS: calculated for C₂₂H₁₇N₇⁺ [M-H]⁺: 380.1618 found: 380.1616.

3-(Dimethylamino)-10-methyl-6-(6-methyl-1,2,4,5-tetrazin-3-yl) acridin-10-ium chloride

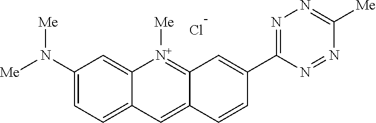 [SMILES: Cc1nnc(-c2ccc3cc4ccc(N(C)C)cc4[n+](C)c3c2)nn1 ; Cl]

PINK (4.50 mg, 14.2 μmol, 1.0 eq) was dissolved in 1 mL toluene and Mel (221 μL, 3.56 mmol, 250 eq) was added. The reaction mixture was heated to reflux in a sealed pressure flask for 19 h. The reaction mixture was centrifuged and the supernatant was decanted. The crude product was loaded onto a DOWEX® 1×4-200 ion exchange resin (700 mg, activated with 1M aq. HCl and equilibrated with H₂O) and eluted with a 1:1 mixture of H₂O/MeOH. Product containing fraction were concentrated by lyophilization. Purification by column chromatography (5→10% MeOH in DCM) yielded XXX (5.00 mg, 13.6 μmol, 96%) as a dark red solid.

¹H NMR (500 MHZ, MeOH-d4): δ [ppm]=9.39 (s, 1H), 9.18 (s, 1H), 8.79 (d, J=8.5 Hz, 1H), 8.46 (d, J=8.5 Hz, 1H), 8.16 (d, J=9.5 Hz, 1H), 7.68 (dd, J=9.6, 2.2 Hz, 1H), 6.88 (s, 1H), 4.51 (s, 3H), 3.50 (s, 6H), 3.14 (s, 3H).

¹³C NMR (125 MHZ, methanol-d₄) δ [ppm]=168.3, 163.3, 157.9, 145.3, 143.8, 140.6, 138.4, 133.4, 132.2, 125.1, 123.9, 122.8, 119.1, 115.4, 91.9, 40.2, 35.7, 19.9.

ESI-HRMS: calculated for C₁₉H₁₉N₆⁺ [M]⁺: 331.1666; found: 331.1651.

3-(Dimethylamino)-6-(6-methyl-1,2,4,5-tetrazin-3-yl)acridin-10-ium chloride (PINK-hydrochloride)

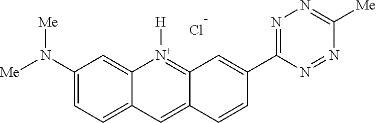 [SMILES: Cc1nnc(-c2ccc3cc4ccc(N(C)C)cc4[nH+]c3c2)nn1 ; Cl]

PINK (5.00 mg, 15.8 μmol, 1.0 eq) was suspended in 600 μL MeCN and HCl (100 mM aq., 600 L, 60 μmol, 3.8 eq) was added, upon which the solution turned purple and a precipitate formed. The supernatant was removed and residual solvent was removed by lyophilization to give PINK-hydrochloride as a dark red solid. No yield was recorded.

¹H NMR (400 MHZ, DMSO-d6): δ [ppm]=15.29 (s, 1H), 9.36 (s, 1H), 9.11 (s, 1H), 8.60 (d, J=8.6 Hz, 1H), 8.47 (d, J=8.6 Hz, 1H), 8.19 (d, J=9.6 Hz, 1H), 7.68 (dd, J=9.6, 2.4 Hz, 1H), 6.91 (s, 1H), 3.34 (s, 6H), 3.08 (s, 3H).

### Example 4—Therapeutic Use

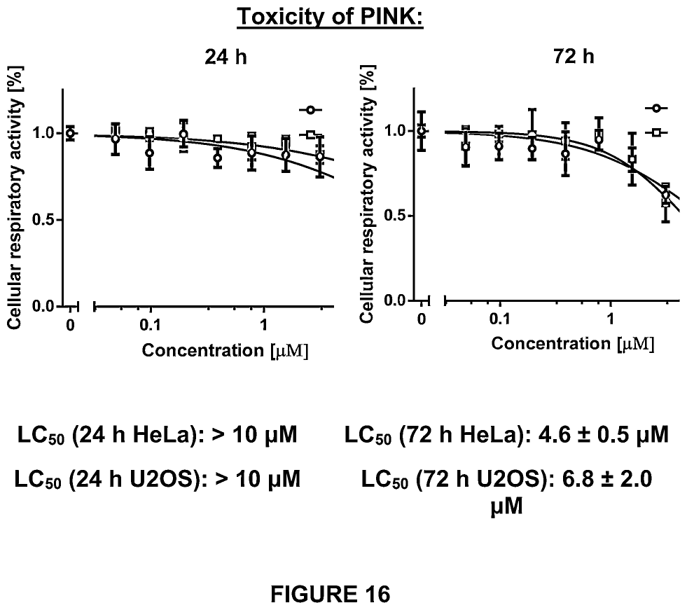

Toxicity studies using resazurin to measure the cellular respiratory activity indicated that low micromolar concentrations of PINK were not toxic to cells even at longer incubation times (FIG. 16). Vinyl nucleosides, such as VdU or VdA, are also rather non-toxic to cells. If cells are exposed to both compounds however, the product of the click reaction introduces a bulky, covalent modification to the DNA. Similar to classical, chemotherapeutic DNA-alkylating agents, such a modification induces genomic instability and can eventually lead to cell death.

A binary chemotherapy approach to form DNA mono adducts using vinyl nucleosides, such as VdU and VdA, and a tetrazine substituted bioorthogonal intercalating reagent, PINK (structures provided below) were investigated. This approach was tested for the treatment of acute myeloid leukemia (AML).

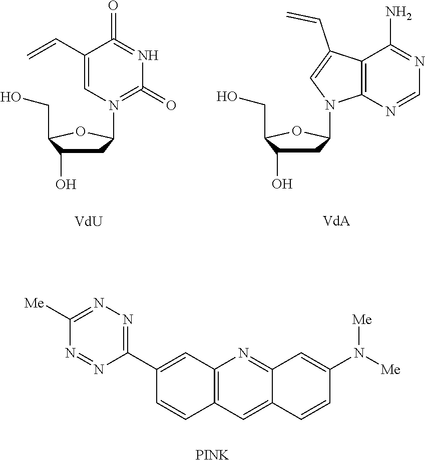

AML is a type of cancer of the blood and bone marrow tissues; it is characterized by the uncontrollable differentiation and proliferation of immature myeloid lineage cells.^{13,14} It is the most common form of acute leukemia in adults.¹⁵ It is a highly heterogeneous cancer, making the diagnosis and treatment of AML challenging.¹³ As a result, AML is classified using the French-American British system (FAB) based on which level the different DNA abnormalities occur in the differentiation level of the myeloid progenitor cell.^{13,16} With MO being at the less differentiated level, which is at the myeloblast and M7 being at the most differentiated level, which is at the megakaryoblast level.^{13,16} Currently, the first line of treatment for AML is the “7+3” days regiment, which consists of 7 days of continuous cytarabine (AraC) infusion followed by 3 days of continuous infusion with an anthracycline, such as daunorubicin or idarubicin.¹³ Unfortunately, this treatment is mostly administered to patients with favorable/intermediate risk prognosis and younger patients since they tend to have lower risk of treatment related mortality.^{13,17} Elderly AML patients who are over 65 years old are usually less tolerant to this type of intensive chemotherapy treatment due to them usually having worse prognosis due to them having worse cytogenetic risk profiles, e.g. FLT3 mutations, and they tend to be more susceptible to treatment-related toxicities. As a result, elderly patients are usually left with no optimal treatment for AML.¹³ By exploring this binary chemotherapy approach to forming DNA mono adducts, using vinyl-nucleosides (VdU/VdA) in combination with PINK for treating AML, the hope is to take advantage of the selectivity of the bioorthogonal reaction to effectively kill the tumors at lower drug concentrations to limit treatment-related toxicities and to provide, in the future, an alternative treatment for patients who are not suitable for the standard “7+3” days regiment of cytarabine and anthracyclines. This binary approach has a lot of potential, since previous research in the Luedtke group have showed VdU to have relatively little toxicity compared to other modified uracil nucleoside, across several cancer cell lines.¹⁸ Furthermore, currently unpublished research in the Luedtke group have showed PINK to be very effective at intercalating into DNA and to have high reaction kinetics with incorporated vinyl-modified nucleosides, such as VdU.¹⁹

Three leukemia cell lines were selected to test this new binary chemotherapy approach for treating AML: MV4-11, MOLM-13 and Jurkat cells. MV4-11 is an AML cell line with the FLT3 mutation, which promotes the excessive growth of abnormal myeloid cells.^{20,21} The FLT3 mutation is present in approximately 20% of AML cases and is associated with unfavorable prognosis.^{20,21} MOLM-13 is another AML cell line derived from AML-M5a, which is known as acute monoblastic leukemia (AMOL).^{22,23} AML-M5a accounts for 5-8% of AML cases.²³ The advantage of using MV4-11 and MOLM-13 is that they are also used as human xenograft models to study AML in mice.²⁴ Jurkat is a T-acute lymphoblastic leukemia (T-ALL) cell line, and it was used to evaluate the specificity of the binary approach in treating AML.²⁵

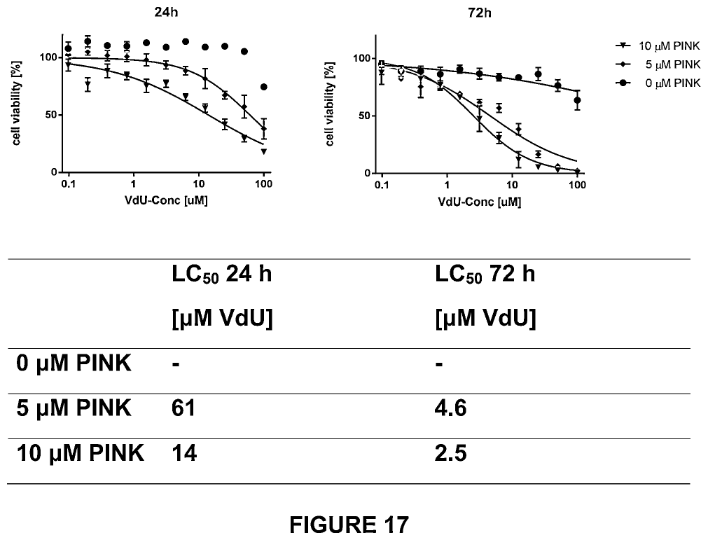

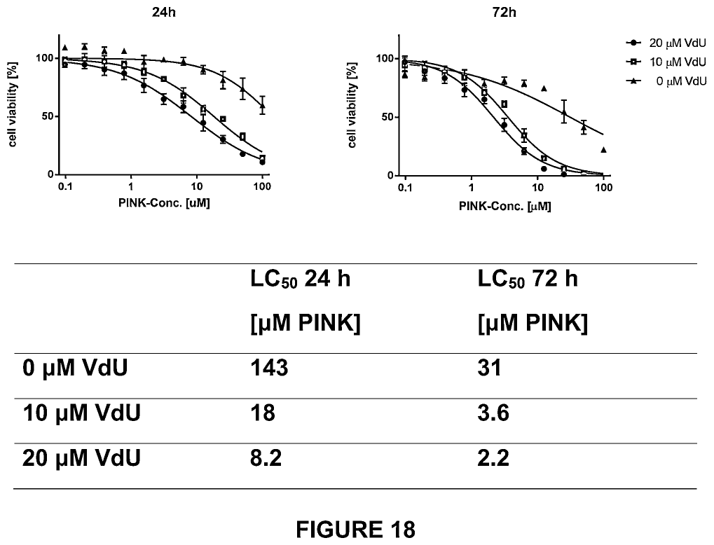

Preliminary experiments were conducted in Hela cells. Cells were treated with increasing concentration of VdU (0-100 μM) for 16 h and subsequently with fixed concentrations of PINK (0, 5, 10 μM) for 5 h. Toxicity was evaluated using an Alamar Blue Assay after 24 and 72 h respectively (FIG. 17). Cells were treated with fixed concentration of VdU (0-10, 20 μM) for 16 h and subsequently with increasing concentrations of PINK (0-100 μM) for 5 h. Toxicity was evaluated using an Alamar Blue Assay after 24 and 72 h respectively (FIG. 18).

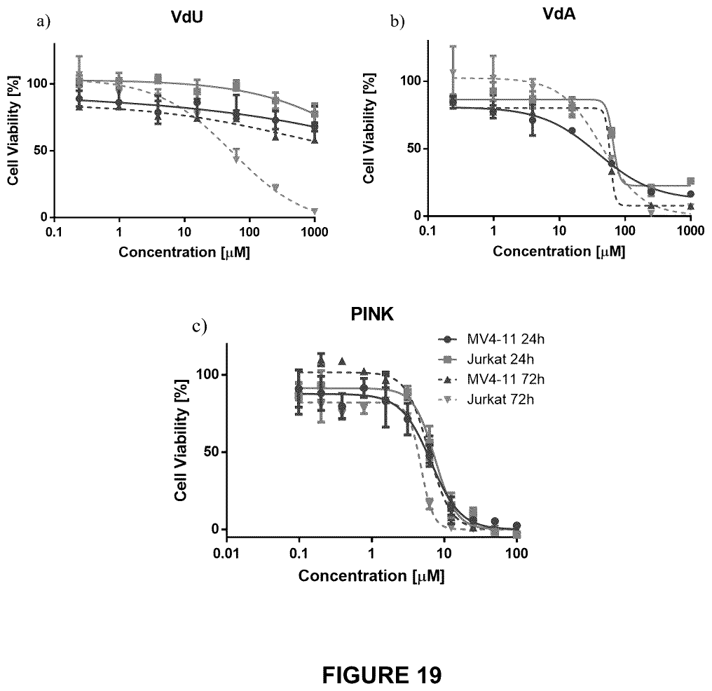

Resazurin assays were carried out to test the effectiveness of VdU, VdA and PINK alone in MV4-11 and Jurkat. The resazurin reduction assay is a colorimetric assay which allows to determine a cell's viability based on its metabolic activities.²⁶ When the cells are alive, the resazurin gets reduced by NADH into its resorufin form by cellular respiration, which is pink and highly fluorescent.²⁶ When the cells are dead or their metabolisms are impaired, the resazurin remains in its oxidized form, which is blue and has low fluorescence.²⁶ The cells can then be imaged using a spectrometer to determine how viable they are after each treatment. Inhibitory dose response curves were generated as shown in FIG. 19. From these dose response curves was found the half maximal inhibitory concentration (IC₅₀) of each compound in the two leukemia cell lines after 24 h and 72h as shown in Table 3.

TABLE 3
IC₅₀ values for VdU, VdA, and PINK after 24 h and 72 h incubation
in MV4-11 and Jurkat cells. The values were calculated
with GraphPad Prism 6 Software using a non-linear regression
(four parameters inhibitory dose response curve) and interpolating
the value at 50% cell viability.

| | IC₅₀ value [μM] (24 h) | | | IC₅₀ value [μM] (72 h) | | |
|---|---|---|---|---|---|---|
| Cell type | VdU | VdA | PINK | VdU | VdA | PINK |
| MV4-11 | >1000 | 32 | 5.7 | >1000 | 58 | 6.2 |
| Jurkat | >1000 | 68 | 7.0 | 52 | 46 | 4.4 |

According to FIG. 19 and Table 3, PINK showed acute toxicity in both MV4-11 and Jurkat, with an averaging IC₅₀ of 5 μM after 24 h and 72h. For the vinyl-nucleosides, VdA showed to have slight acute toxicity after 24 h and 72h with an IC₅₀ in the range of roughly 50 μM. VdU showed to be relatively non-toxic in both cell lines with IC₅₀ above 1000 μM, except for Jurkat where it showed some slight toxicity after 72h (IC₅₀=52 μM). Overall, VdU presented itself as a good candidate for the combination therapy with PINK due to its low toxicity in the two leukemia cell lines.

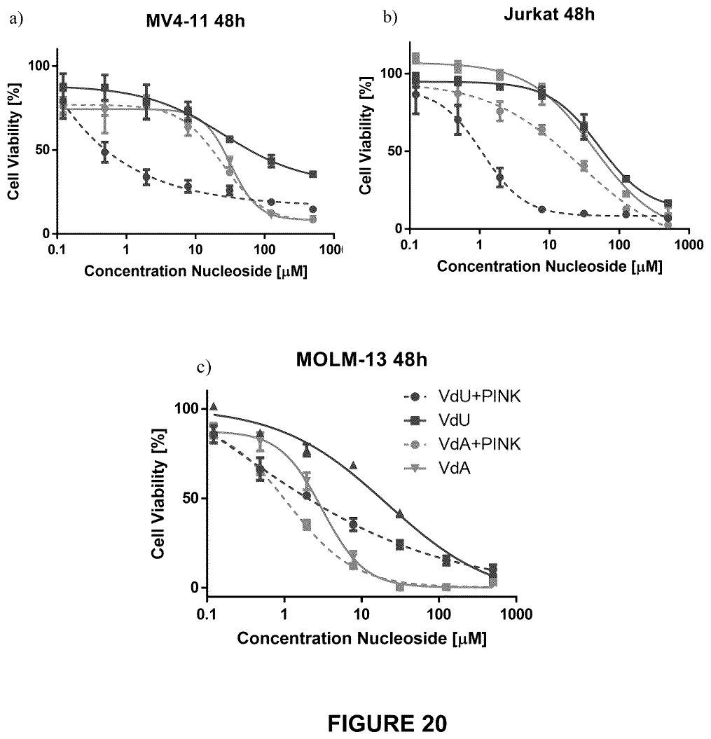

From the single toxicity experiment, it was determined that the optimal subtoxic concentration of PINK to be used for the combination toxicity assays would be 1 μM. To test how effective the vinyl-nucleosides (VdU/VdA) in combination with PINK are at killing the cells compared to the vinyl-nucleosides alone, another resazurin reduction assay was carried out. Inhibitory dose response curves with the corresponding IC₅₀ for both the combinations and the vinyl-nucleosides alone in MV4-11, Jurkat, and MOLM-13 after 48h were generated, as shown in FIG. 20 and Table 4, respectively.

TABLE 4
IC50 values for VdU/VdA alone and in combinations with
1▭M of PINK (added 24 h post nucleoside addition) after
48 h incubation in MV4-11, Jurkat, and MOLM-13 cells. The
values were calculated with GraphPad Prism 6 software
using a non-linear regression (four parameters inhibitory dose
response curve) and interpolating the value at 50% cell viability.

| | IC₅₀ value [μM] (48 h) for sequential addition | | | |
|---|---|---|---|---|
| Cell type | VdU + PINK | VdU | VdA + PINK | VdA |
| MV4-11 | 0.50 | 60 | 18 | 25 |
| Jurkat | 1.1 | 58 | 17 | 46 |
| MOLM-13 | 2.0 | 18 | 0.99 | 2.6 |

According to FIG. 20, a large difference in cell viability between VdU alone and VdU in combination can be seen with as little as 1 μM of PINK in MV4-11 and Jurkat. As illustrated by Table 4, the IC₅₀ values of VdU+PINK in MV4-11 and Jurkat was 0.50 μM and 1.1 μM, respectively, which is significantly less than the IC₅₀ of VdU alone in MV4-11 (60 μM) and Jurkat (58 μM). This shows that the VdU+PINK combination is much more effective at killing these two cell lines than VdU alone. A similar toxicity trend for VdU+PINK was observed in MOLM-13, even though at lesser extent than in MV4-11 and Jurkat cells, where the IC₅₀ for VdU+PINK and VdU alone was 2.0 μM and 18 μM, respectively. This demonstrates that MOLM-13 seems to be more susceptible to VdU on its own than the other two leukemia cell lines. For VdA+PINK, little to no difference was seen in terms of cell viability compared to VdA alone. MOLM-13 also showed to be more susceptible to VdA alone (IC₅₀=3 μM) compared to MV4-11 (IC₅₀=25 μM) and Jurkat (IC₅₀=46 μM). The little to no difference in cell viability between VdA and VdA+PINK indicates that VdA probably doesn't get incorporated into the DNA, as opposed to VdU. As a result, VdA might be inducing cytotoxicity by a different mechanism than VdU, such as by depleting the ATP pool of the cell, as it was previously reported for other modified adenosine nucleosides.²⁷

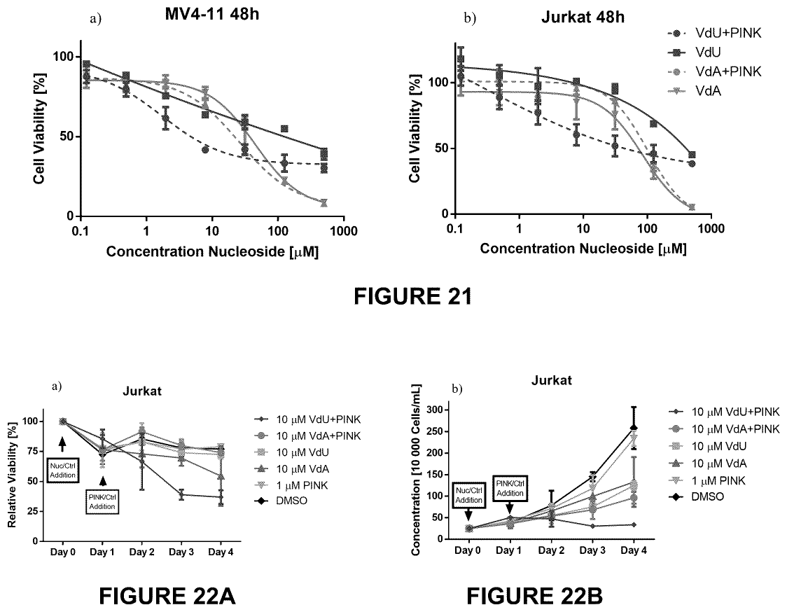

To determine if it is possible to treat the leukemia cells with the vinyl-nucleosides and PINK simultaneously, instead of adding them sequentially, another resazurin reduction assay using the same concentrations of each compound as in the previous combination experiment was carried out, where instead the vinyl-nucleosides and PINK were added on the same day instead of on two consecutive days. Inhibitory dose response curves were generated with the corresponding IC₅₀ values for the vinyl-nucleosides alone or in combination with 1 μM PINK in MV4-11 and Jurkat after 48h of incubation, as shown in FIG. 21 and Table 5, respectively.

TABLE 5
IC₅₀ values for VdU/VdA, alone and in combination with 1 μM
of PINK (nucleoside and PINK added on the same day)
after 48 h incubation in MV4-11, Jurkat cells. The values were
calculated with GraphPad Prism 6 software using a non-linear
regression (four parameters inhibitory dose response
curve) and interpolating the value at 50% cell viability.

| | IC₅₀ value [μM] (48 h) for simultaneous addition | | | |
|---|---|---|---|---|
| Cell type | VdU + PINK | VdU | VdA+PINK | VdA |
| MV4-11 | 4.7 | 124 | 22 | 37 |
| Jurkat | 44 | 404 | 97 | 72 |

From FIG. 21, it was observed that VdU+PINK was less effective at killing MV4-11 and Jurkat cells when VdU and PINK were added simultaneously instead of sequentially (FIG. 20). Furthermore, as demonstrated by Table 5, the IC₅₀ values of VdU+PINK in MV4-11 (4.7 μM) and Jurkat (44 μM) were much larger when VdU and PINK were added simultaneously, than when they were added sequentially in MV4-11 (0.50 μM) and Jurkat (1.1 μM) (Table 4). The simultaneous addition of the two compounds might be less effective at killing the cells due to two reasons: 1) VdU and PINK might react before incorporation of VdU, which would not induce DNA alkylation, and 2) because PINK might get cleared from the cells before it can react with VdU to alkylate DNA. Interestingly, VdA in combination with PINK still had approximately the same level of toxicity as VdA alone in both simultaneous and sequential additions. This further confirms what was observed in FIG. 20 and Table 4 and supports the hypothesis that VdA doesn't get incorporated into the DNA and instead induces cytotoxicity by another mechanism than VdU+PINK; potentially, by the inhibition of enzymes involved in the synthesis of ATP resulting in the depletion of the ATP pool of the cell.

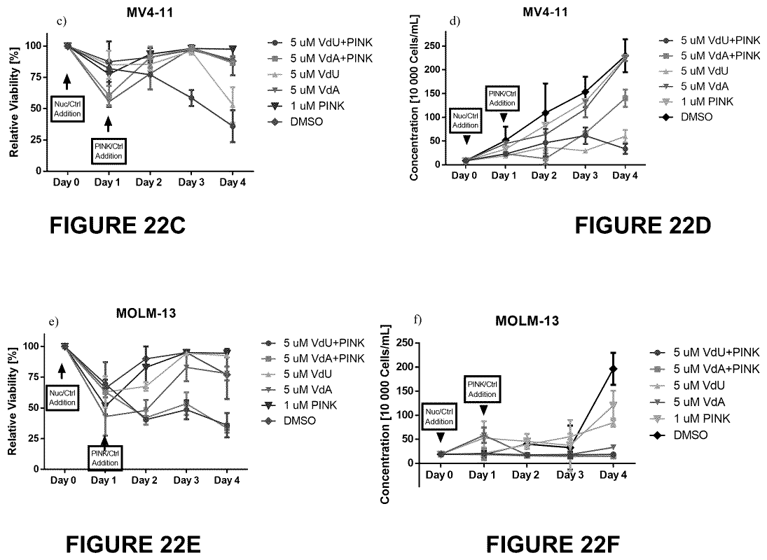

To confirm the cytotoxicity results obtained from the resazurin assays, a cell counting assay was conducted using Trypan Blue. It is an azo dye allows to discriminate between viable cells and dead cells, since dead cells will take in the dye, while viable cells will remain white. 28 From the resazurin assays, and after some optimization based on cell sensitivity to the vinyl-nucleosides, it was determined that 10 M and 5 μM of vinyl-nucleosides alone and in combination with 1 μM of PINK were two interesting concentrations to explore in Jurkat, MV4-11, and MOLM-13 cells. Cells were incubated with 5 μM or 10 μM of each nucleoside overnight (Day 0), and 1 μM of PINK was added for the combinations and PINK alone 24h post nucleoside incubation (Day 1). The cells were counted every 24h. Graphs for relative cell viability and concentration of cells after each consecutive day of incubation with their respective treatments were generated, as seen in FIG. 22A-F.

From FIG. 22A, as expected from the resazurin assay, 10 μM VdU in combination with 1 μM of PINK was more effective at killing the Jurkat cells than VdU alone, as seen after Day 3 (48h combination incubation). In contrast, 10 μM of VdA in combination with 1 μM of PINK had relatively low toxicity in Jurkat cells, similarly to VdA alone, as illustrated by the high relative viability of Jurkat cells. From FIG. 22B, it was observed that 10 μM VdU+PINK was very effective at inhibiting cell growth. This indicates that the combination of VdU and PINK seems to cause cell death in Jurkat cells, as it was expected from the resazurin assay. Interestingly, VdU alone had more impact on cell growth than expected from the resazurin assay, which suggests that VdU by itself might be causing cell cycle arrest, but when combined with PINK it would induce cell death.

In MV4-11 cells, according to FIG. 22C, it was observed that 5 μM of VdU in combination with 1 μM of PINK was much more effective at killing the cells than VdU alone, as seen after Day 3. In contrast, 5 μM of VdA in combination with 1 μM of PINK was as ineffective at killing the cells as VdA alone. From FIG. 22D, it was noted that 5 μM VdU+PINK was very effective at inhibiting cell growth in MV4-11. This suggests that VdU in combination with PINK induces cell death in MV4-11. Surprisingly, VdU significantly inhibited cell growth, which signifies that VdU by itself might be inducing cell cycle arrest in MV4-11 cells. Furthermore, VdA in combination with PINK inhibited cell growth as well, which suggests that VdA+PINK might be inducing cell cycle arrest in MV4-11.

In MOLM-13 cells, according to FIG. 22E, it was observed that 5 μM of VdU in combination with 1 μM of PINK was much more effective at killing the cells than VdU alone, as seen after Day 3. Interestingly, 5 μM of VdA in combination with 1 M of PINK was much more effective at killing the cells than VdA alone. From FIG. 22F, it was noted that the combinations of VdU with PINK and VdA with PINK were both significantly inhibiting cell growth. This indicates that both VdU+PINK and VdA+PINK seem to induce cell death. Interestingly, both VdU and VdA seem to inhibit cell growth, which indicates that VdU and VdA by themselves might be inducing cell cycle arrest in MOLM-13.

Overall, in all three cell lines the combination of VdU with PINK seemed to induce cell death. In contrast, the combination of VdA with PINK seemed to induce cell cycle arrest in Jurkat and MV4-11, but cell death in MOLM-13. Interestingly, in all three leukemia cell lines it was noted that VdU alone seemed to induce cell cycle arrest, but when it was combined with PINK it induced cell death. More cell counting experiments would need to be conducted at smaller/more optimized nucleoside concentrations to confirm these findings. Nevertheless, the dual cytotoxicity mechanism of VdU would be an interesting therapeutic feature to explore in AML cell lines which are resistant to AraC treatment due to an overexpression of cytidine deaminase, since it converts the cytosine base into an uracil base, which renders the drug ineffective at killing the tumors, probably due to it inducing cell cycle arrest.²⁹ Going forward, this resistance mechanism could potentially be utilized for treating AML patients who develop resistance to AraC treatment. This could be done by first administering VdC, which by itself is fairly non-active, but it might get transformed into VdU, by the cytidine deaminases, and then get incorporated into DNA, where it could react with PINK to alkylate DNA and induce cell death.

Lastly to determine how synergistic the combination of VdU with PINK is in the three leukemia cell lines, another resazurin reduction assay was conducted where VdU and PINK were maintained at a constant 5:1 ratio. The data generated from this resazurin assay were analyzed using the synergy model developed by Chou and Talalay 30, which is based on the median effect dose (IC₅₀) and the slope of the drug response curve, as shown in equation II:

$$CI = \frac{{( D )}_{1}}{{( {D}_{x} )}_{1}} + \frac{{( D )}_{2}}{{( {D}_{x} )}_{2}} = \frac{{( D )}_{1}}{{{( {D}_{m} )}_{1} [ {f}_{a} / ( 1 {sf}_{a} ) ]}^{1 / m 1}} + \frac{{( D )}_{2}}{{{( {D}_{m} )}_{2} [ {f}_{a} / ( 1 - {f}_{a} ) ]}^{1 / m 2}} \qquad ( Equation II )$$

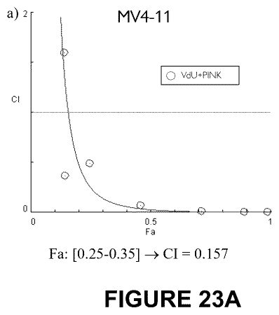

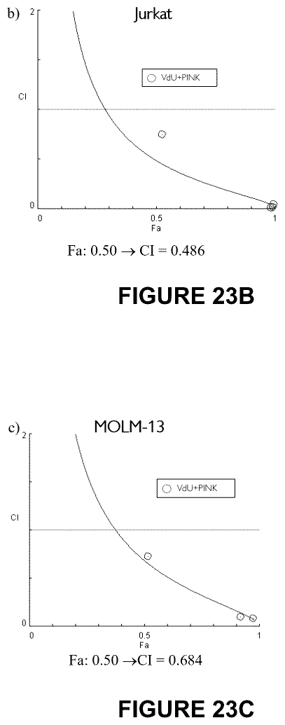

The model allows to quantitatively measure the synergy of drug combinations, by generating a combination index (CI).³⁰ A CI below 1 indicates a synergistic interaction which means that the effect of the drug combination is greater than the effect of both drugs alone combined.³⁰A CI equal to 1 indicates an additive interaction which means that the effect of the drug combination is equal to the effect of both drugs alone combined.³⁰ A CI above 1 indicates an antagonistic interaction which means that the effect of the drug combination is less than the effect of both alone combined. 30 Fa-CI plots were generated for the VdU+PINK combination in MV4-11, Jurkat and MOLM-13, where the CI at specific fraction of affected cells (Fa) was identified for each cell lines, as seen in FIG. 23A-C. Furthermore, a dose reduction index (DRI) was generated for each compound and was identified at specific Fa for each cell lines, as seen in Table 6. Where a DRI above 1 is a favorable dose reduction and a DRI below 1 is an unfavorable dose reduction.³⁰

TABLE 6
Dose Reduction Index (DRI) at specific fraction of affected cells(Fa)
for VdU and PINK in MV4-11, Jurkat and MOLM-13 cells when
treated with VdU + PINK combination at constant 5:1 ratio.

| | | Concentration | Concentration | DRI | DRI |
|---|---|---|---|---|---|
| Cell type | Fa | VdU [μM] | PINK [μM] | VdU | PINK |
| MV4-11 | [0.25-0.35] | 59 | 3.2 | 34-fold | 10-fold |
| Jurkat | 0.50 | 3.7 | 1.1 | 3-fold | 5-fold |
| MOLM-13 | 0.50 | 4.2 | 1.2 | 2-fold | 4-fold |

From FIG. 23A, it was observed that VdU in combination with PINK was highly synergistic in MV4-11, since at a significant Fa range of 0.25 to 0.35, where 25% to 35% of the cells were killed, the CI=0.157. In FIG. 23B, great synergy between VdU and PINK was observed as well in Jurkat cells, but at a lesser extent than in MV4-11. At a significant Fa of 0.50, (50% cells killed) the CI=0.486. According to FIG. 23C, VdU+PINK is the least synergistic in MOLM-13, where at a significant Fa of 0.50, the CI=0.684.

From Table 6, it was noted that to kill 25% to 35% of MV4-11 with VdU alone, a concentration of 59 μM would be required; whereas, with PINK alone it would require 3.2 μM. With the VdU+PINK combination, the concentration of VdU and PINK given can be reduced by a factor of 34 and 10, respectively. To kill 50% of the Jurkat cells with VdU alone, a concentration of 3.7 μM would be required; whereas, with PINK alone it would require 1.1 μM. With the VdU+PINK combination, the concentration of VdU and PINK given can be reduced by a factor of 3 and 5, respectively. Finally, to kill 50% of MOLM-13 cells with VdU alone, a concentration of 4.2 μM would be required; whereas, with PINK alone it would require 1.2 μM. With the VdU+PINK combination, the concentration of VdU and PINK given can be reduced by a factor of 2 and 4, respectively.

Overall, the combination of VdU with PINK was found to have significant synergism across all three cell lines, with the most in MV4-11 and the least in MOLM-13. Compared to other combination treatments, such as AraC with Aplidin, which were tested for the treatment of different types of acute lymphoblastic leukemia (ALL), the synergism of VdU with PINK in MV4-11 is significantly more prominent, and for Jurkat it is equal to the synergism of AraC+Aplidin in the ALL cell lines tested.^{31,32} Furthermore, the highly synergistic interaction between VdU and PINK contributes to great DRI observed across all three leukemia cell lines tested, with MV4-11 having highest DRI and MOLM-13 having the lowest DRI for both VdU and PINK. As a result, the use of VdU in combination with PINK could potentially be beneficial in terms of decreasing treatment-related toxicities by reducing the dose of each compound needed to effectively kill tumors clinically.

From these findings, VdU in combination with PINK presents itself as a good candidate for the binary chemotherapy approach for AML and T-ALL. This is reflected by the great synergism of VdU with PINK in all three leukemia cell lines tested, since VdU alone is relatively non-toxic. As well, the high DRI of the combination allows to reduce the dose of PINK and VdU given, while achieving important cell killing, which might help reduce treatment-related toxicities clinically. Going forward, the combination of VdU with PINK should be tested in MV4-11 and MOLM-13 xenograft models, and in other AML cell lines. Some additional experiments should be done as well to confirm the findings, such as cell counting experiments with more optimized nucleoside concentrations, Western blots to screen for DNA damage markers and cell cycle analysis experiments via flow cytometry. In contrast, VdA in combination with PINK appears to be a less effective candidate for the binary chemotherapy approach for AML and T-ALL. This is reflected by the low level of synergy between VdA and PINK, and by the fact that VdA by itself is significantly more toxic than VdU. Furthermore, the combination of VdA with PINK doesn't seem to be effective at killing the leukemia cells tested, since it likely induced cell cycle arrest in both MV4-11 and Jurkat cells.

### Experimental Section

Cell Culture. MV4-11, MOLM-13 and Jurkat cells were cultured at 37° C., 5% CO₂ in RPMI supplemented with 10% FBS, 1% MEM non-essential amino acid solution and 1% penicillin-streptomycin. Cells were grown to confluency and passaged every 2 days (MV4-11) and 3 days (MOLM-13 and Jurkat) in a 1:10 split. Cells were counted using BIO RAD TC20 cell counter to determine the seeding densities.

Resazurin Single toxicity assay. MV4-11 and Jurkat cells were seeded in 96-well plates at a density of 20 000 cells/well, in triplicates. The cells were treated with each nucleoside (VdU/VdA) alone diluted in fresh media in 1:4 serial dilutions (˜0.01% DMSO) at concentrations of 1000-0 μM. The cells were treated with PINK alone diluted in fresh media in 1:2 serial dilutions (˜1% DMSO) at concentration of 100-0 μM. The cells were then incubated for 24 h and 72h. 10 μL of 870 μM resazurin was added per well, and incubated for 2-5 hours. After incubation, the fluorescence intensity at 590 nm (excitation=560 nm) was measured using a SpectraMax M5 plate reader.

Resazurin Combination toxicity assays. MV4-11, MOLM-13 and Jurkat cells were seeded in 96-well plates at a density of 20 000 cells/well, 25 000 cells/well and 30 000 cells/well, respectively, in triplicates. For the sequential addition, the cells were first treated with the nucleosides (VdU/VdA) diluted in fresh media in 1:4 serial dilutions (˜0.01% DMSO) at concentrations of 500-0 μM, and incubated overnight. 24h post nucleoside addition, the cells were treated with 1 μM PINK diluted in fresh media (˜1% DMSO), and incubated for 24-72h. The cells were treated with resazurin, incubated and measured, as described above. For the simultaneous addition, the cells were treated with nucleosides and PINK on the same day at the same concentrations as describes above, and incubated for 24-72h.

Resazurin Chou-Talalay optimized toxicity assay. MV4-11, MOLM-13 and Jurkat cells were seeded in 96-well plates at a density of 20 000 cells/well, 25 000 cells/well and 30 000 cells/well, respectively, in triplicates. The cells were first treated with VdU diluted in fresh media in 1:4 serial dilutions (˜0.01% DMSO) at concentrations of 500-0 μM alone and in combination, and incubated overnight. 24h post nucleoside addition, the cells were treated with PINK diluted in fresh media in 1:4 serial dilutions (˜1% DMSO) at concentrations of 100-0 μM alone or in combination, and incubated for 72 h. The combinations had a constant 5:1 ratio of VdU to PINK. The cells were treated with resazurin, incubated and measured, as described above. The data was processed and analysed in the CompuSyn program used for running Chou-Talalay synergy analyses.³³

Cell Counting Assay. Jurkat, MV4-11 and MOLM-13 cells were seeded in 12-well plates at a density of 500 000 cells/mL, 175 000 cells/mL and 375 000 cells/mL, respectively, in duplicates. The cells were incubated overnight in fresh media containing 10 μM (Jurkat) or 5 μM (MV4-11 and MOLM-13) of each nucleoside (˜0.01% DMSO). 24h post nucleoside addition, 1 μM of PINK (˜0.01% DMSO) were added to the respective wells and incubated for 24 h. The cells were counted using a cell counter (BIO RAD TC20) to determine the percentage of viable cells and the cell concentration. The cells were counted every 24h using Trypan Blue in a 1:1 ratio.

### Example 5—5-VdU and PINK Combination Studies

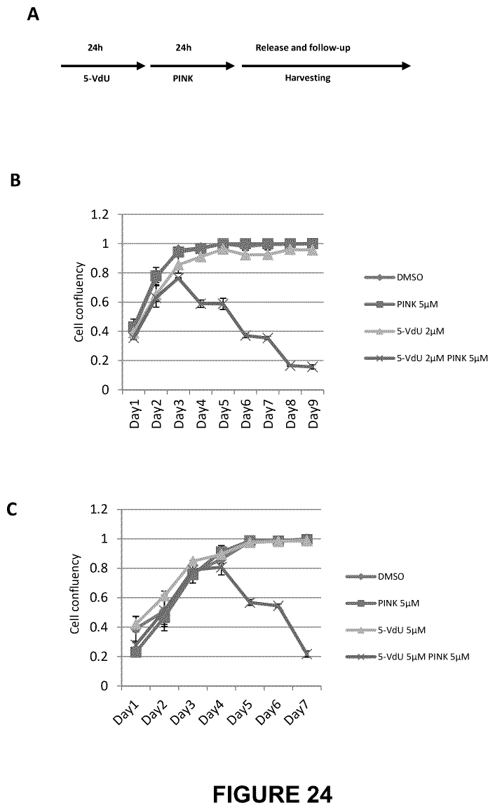

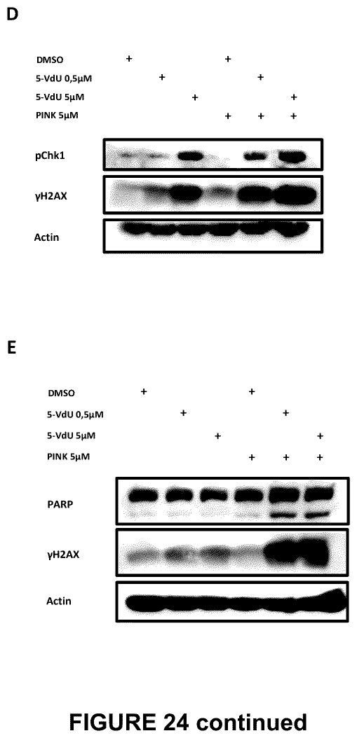

Studies on the 5-VdU (VdU) and PINK combinatory impact on cells were conducted (see FIGS. 24-32). The 5-VdU/PINK combination works in two additional cell lines, H1299 a human lung cancer cell line and KPC a mouse pancreatic cell line. These studies provide that the compounds show functionality in cell lines derived from two additional organs and one additional species.

Synergistic lethality effects of 5-VdU and PINK were shown in a confluency growth assay with H1299 cells and mouse KPC cells, while Western Blot analysis revealed synergistic effects of 5-VdU and PINK on DNA damage and cell death markers in these cells. Experiments were conducted in technical triplicates (see FIG. 24).

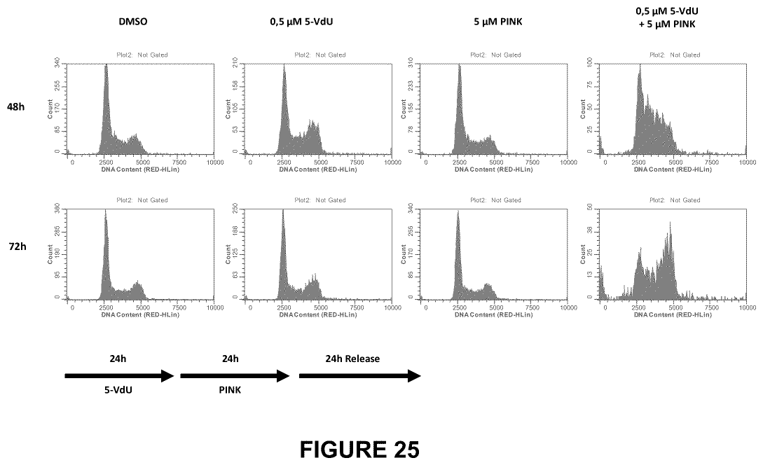

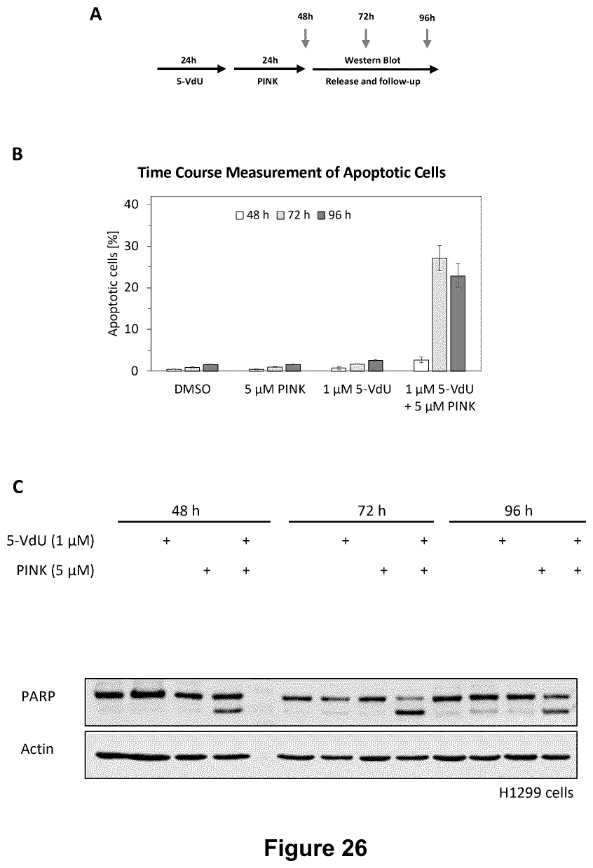

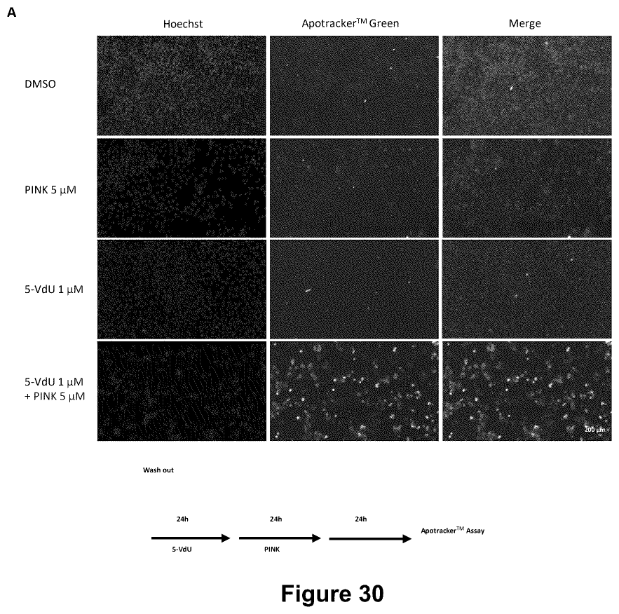

5-VdU and PINK combinatory impact on the cell cycle. FACS analysis of H1299 cells treated with 5-VdU/PINK whos that the cell cycle progression is severely impacted upon combinatory treatment. Time course cell cycle analyses of H1299 cells show mostly unperturbed cell cycle profiles upon treatment with 5-VdU or PINK alone. In the combination of the two drugs, an accumulation of S-phase cells at 48 h and an accumulation of cells at the G2/M boundary at 72 h after the initial treatment were observed (see FIG. 25). Synergistic lethality of 5-VdU and PINK were shown in H1299 cells and the 5-VdU/PINK induces apoptosis with a peak at 72 h post start of treatment. (see FIG. 26). Percentage of H1299 cells undergoing apoptosis was indicated by staining with Apotracker™ Green, induced after treatment with 1 μM 5-VdU and 10 μM PINK for different time points. H1299 cells were seeded at a density of 20% and treated with 5-VdU and PINK in the indicated concentrations. One day after the last treatment, the medium was exchanged. The cells were then stained with the Apotracker™ Green dye and Hoechst. Images were taken 72h after the first treatment. Acquired pictures at 72h are shown in FIG. 30. (C) H1299 cells were treated with 1 μM 5-VdU for 24 h and subsequently with 5 μM PINK for another 24 h.

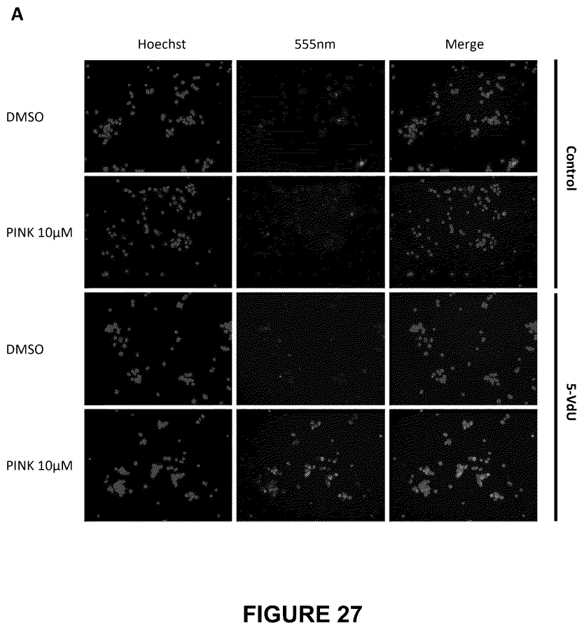

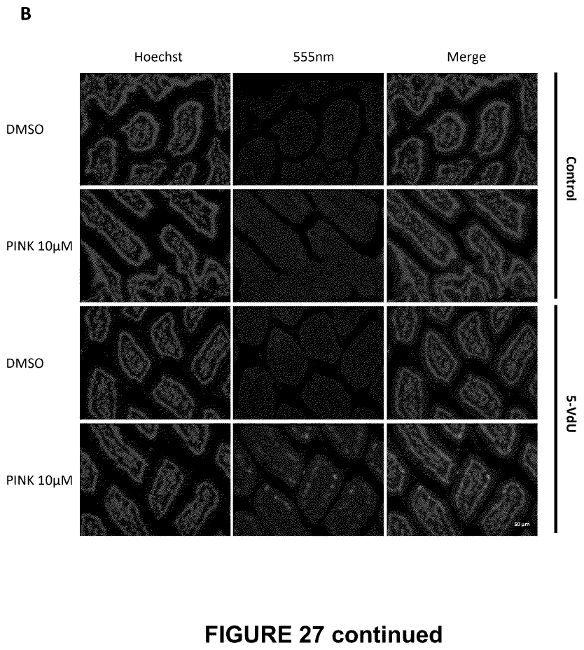

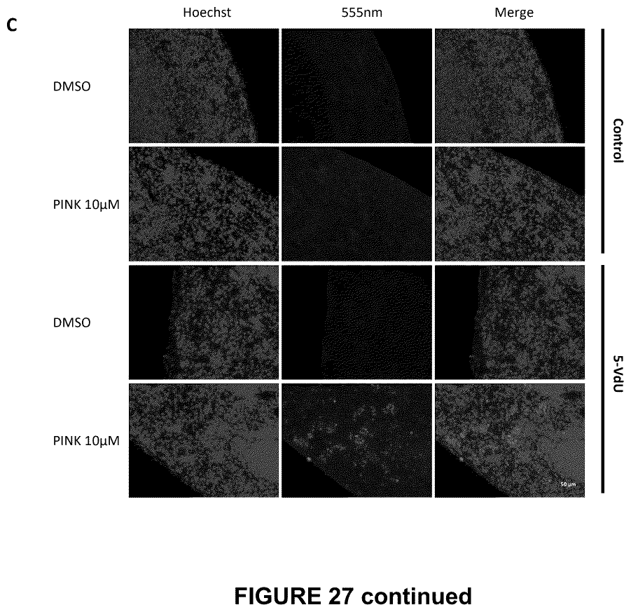

5-VdU incorporation in rapidly proliferating cells in vivo. C57BL/6J mice were either injected with 200 μl 10% DMSO in 1× sterile PBS as a vehicle or 500 mg/kg 5-VdU i.p. on two subsequent days and then sacrificed. Experimental group size was n=6. (A) Bone marrow was explanted, fixed, permeabilized and stained with PINK for 4 h at RT. Nuclei were counterstained with Hoechst33342. A strong nuclear signal can be observed in the PINK treated sample when compared to the controls. (B) Small intestine and (C) spleen tissue was subjected to paraffin embedding and the slides where then stained with PINK for 4 h at RT. Nuclei were counterstained with Hoechst33342. Specific nuclear signals are visible in the PINK treated sample when compared to the controls. (See FIG. 27) 5-VdU is non-toxic to mice at high concentrations and incorporates into rapidly dividing cells in vivo. These cells can be stained with PINK in FFPE sections=in vivo DNA replication/proliferation reporter.

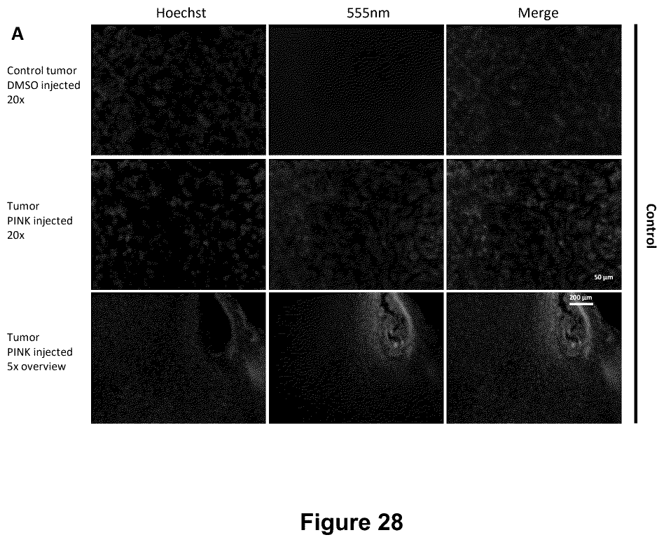

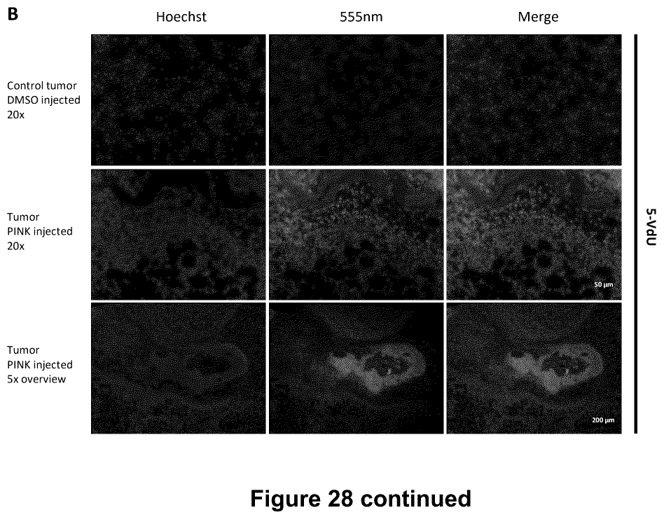

5-VdU/PINK Click Chemistry reaction in mouse xenografts. KPC subcutaneous tumors were generated in both flanks of a syngenic C57BL/6J mouse model. Upon a tumor size of approximately 200 mm3, animals were either injected with 200 μl 10% DMSO in 1× sterile PBS as a vehicle or 500 mg/kg 5-VdU i.p. on two subsequent days and the tumors were then intratumorally injected with either 100 μl 5% DMSO, 30% PEG400 in 1× sterile PBS as a vehicle or 20 mg/kg PINK, each into the left or right tumor of the same animal for an internal control (FIG. 30, B). Experimental group size was n=3. Animals were sacrificed 6h post injection and the explanted tumors were subjected to paraffin embedding. Nuclei were counterstained with Hoechst33342. The PINK reservoir can be identified in the 5× magnification. Specific nuclear signals could be detected only in the 5-VdU injected, but not in the DMSO injected animals. (See FIG. 28) Results show that tumors of 5-VdU treated mice show positive nuclei after PINK intratumoral injection. The reaction takes place in vivo.

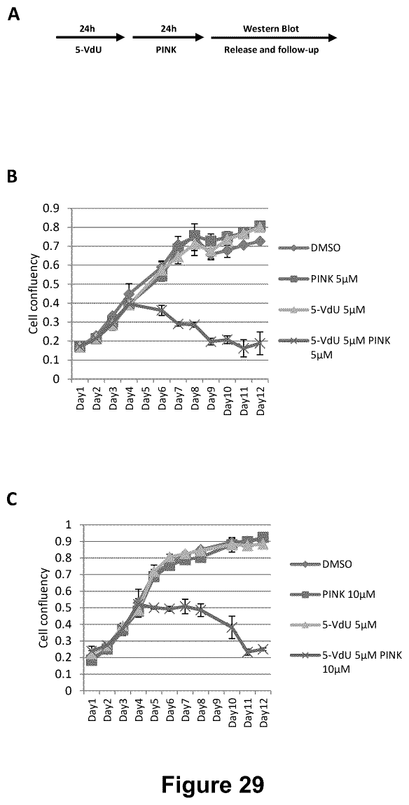

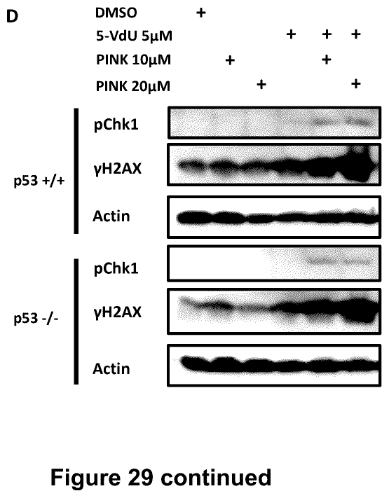

5-VdU/PINK combinatory effects are independent of cellular p53 mutation status. Treatment schematics are shown in FIG. 29. 5-VdU/PINK induced synthetic lethality in HCT116 p53+/+ cells (B) as well as in the isogenic HCT116 p53−/− cell line (C). Experiments were conducted in technical triplicates. (D) Western Blot analysis shows exacerbation of pChk1 and yH2AX when treated with 5-VdU/PINK in both cell lines.

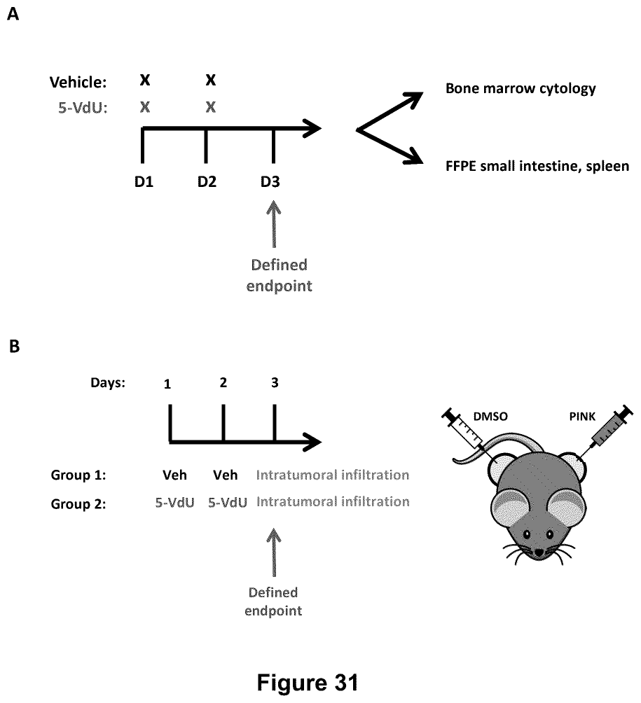

In vivo treatment schematics are shown in FIG. 31.

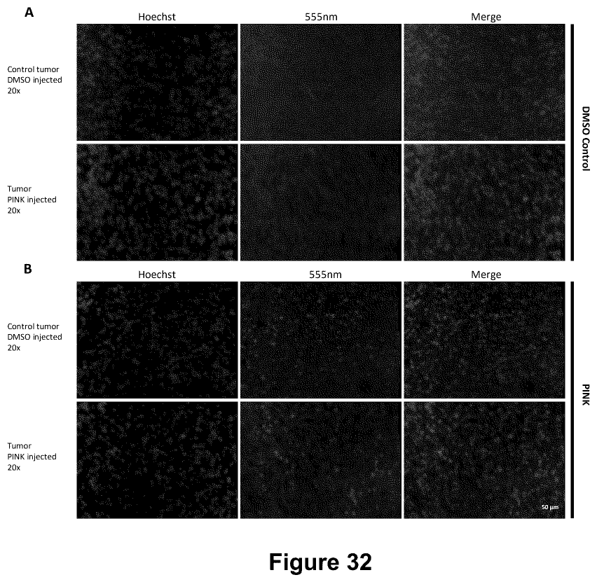

Incorporation of 5-VdU in mouse xenograft cells as revealed by incubation of tissue slices with PINK. KPC subcutaneous tumors were generated in both flanks of a syngenic C57BL/6J mouse model. Upon a tumor size of approximately 200 mm3, animals were either injected with 200 μl 10% DMSO in 1× sterile PBS as a vehicle or 500 mg/kg 5-VdU i.p. on two subsequent days and the tumors were then intratumorally injected with either 100 μl 5% DMSO, 30% PEG400 in 1× sterile PBS as a vehicle or 20 mg/kg PINK, each into the left or right tumor of the same animal for an internal control (FIG. 30, B). Experimental group size was n=3. Animals were sacrificed 6h post injection and the explanted tumors were subjected to paraffin embedding. Slides were stained with DMSO or PINK for 4 h at RT, nuclei were then counterstained with Hoechst33342. Both DMSO and PINK injected tumors showed specific nuclear signal when stained with PINK (B), but not with DMSO (A), suggesting an incorporation of 5-VdU into tumor cell DNA (see FIG. 32). Results support that tumors can be efficiently labeled with 5-VdU, allowing for use of PINK in fluorescence-based tumor localization in cancer surgery.

Although the embodiments have been described in detail, it should be understood that various changes, substitutions and alterations can be made herein. Moreover, the scope of the present application is not intended to be limited to the particular embodiments or examples described in the specification. As can be understood, the examples described above and illustrated are intended to be exemplary only.

For example, the present invention contemplates that any of the features shown in any of the embodiments described herein, may be incorporated with any of the features shown in any of the other embodiments described herein, and still fall within the scope of the present invention.

### References—Example 1

[1] T. Caneque, S. Müller, R. Rodriguez, Nat. Rev. Chem. 2018, 2, 202-215.
[2] E. Kim, H. Koo, Chem. Sci. 2019, 10, 7835-7851.
[3] K. S. Chandrasekaran, A. Rentmeister, Biochemistry 2019, 58, 24-30.
[4] J. Kaur, M. Saxena, N. Rishi, Bioconjug. Chem. 2021, 32, 1455-1471.
[5] H. Wu, B. T. Cisneros, C. M. Cole, N. K. Devaraj, J. Am. Chem. Soc. 2014, 136, 17942-17945.
[6] T. Santner, M. Hartl, K. Bister, R. Micura, Bioconjug. Chem. 2014, 25, 188-195.
[7] T. Lauria, C. Slator, V. McKee, M. Müller, S. Stazzoni, A. L. Crisp, T. Carell, A. Kellett, Chem.-Eur. J. 2020, 26, 16782-16792.
[8] I.-H. Wang, M. Suomalainen, V. Andriasyan, S. Kilcher, J. Mercer, A. Neef, N. W. Luedtke, U. F. Greber, Cell Host Microbe 2013, 14, 468-480.
[9] A. B. Neef, L. Pernot, V. N. Schreier, L. Scapozza, N. W. Luedtke, Angew. Chem. Int. Ed. 2015, 54, 7911-7914.
[10] I. Haralampiev, M. Schade, J. Chamiolo, F. Jolmes, S. Prisner, P. T. Witkowski, M. Behrent, F. Hövelmann, T. Wolff, O. Seitz, A. Herrmann, ChemBioChem 2017, 18, 1589-1592.
[11] D.-L. Leone, M. Hubálek, R. Pohl, V. Sýkorová, M. Hocek, Angew. Chem. Int. Ed. 2021, 60, 17383-17387.
[12] S. Nainar, B. J. Cuthbert, N. M. Lim, W. E. England, K. Ke, K. Sophal, R. Quechol, D. L. Mobley, C. W. Goulding, R. C. Spitale, Nat. Methods 2020, 17, 311-318.
[13] T. Triemer, A. Messikommer, S. M. K. Glasauer, J. Alzeer, M. H. Paulisch, N. W. Luedtke, Proc. Natl. Acad. Sci. 2018, 115, E1366.
[14] A. Messikommer, K. Seipel, S. Byrne, P. J. M. Valk, T. Pabst, N. W. Luedtke, ACS Pharmacol. Transl. Sci. 2020, 3, 1225-1232.
[15] E. M. Sletten, C. R. Bertozzi, Angew. Chem. Int. Ed. 2009, 48, 6974-6998.
[16] C. S. Mckay, M. G. Finn, Chem. Biol. 2014, 21, 1075-1101.
[17] J. T. George, S. G. Srivatsan, Chem. Commun. 2020, 56, 12307-12318.
[18] D. Ganz, D. Harijan, H.-A. Wagenknecht, RSC Chem. Biol. 2020, 1, 86-97.
[19] N. Klöcker, F. P. Weissenboeck, A. Rentmeister, Chem. Soc. Rev. 2020, 49, 8749-8773.
[20] K. Krell, D. Harijan, D. Ganz, L. Doll, H.-A. Wagenknecht, Bioconjug. Chem. 2020, 31, 990-1011.
[21] S. Nainar, S. Beasley, M. Fazio, M. Kubota, N. Dai, I. R. Corrêa Jr., R. C. Spitale, ChemBioChem 2016, 17, 2149-2152.
[22] Y. Zhang, R. E. Kleiner, J. Am. Chem. Soc. 2019, 141, 3347-3351.
[23] J. M. Holstein, L. Anhäuser, A. Rentmeister, Angew. Chem. Int. Ed. 2016, 55, 10899-10903.
[24] H. Liu, T. Ishizuka, M. Kawaguchi, R. Nishii, H. Kataoka, Y. Xu, Bioconjug. Chem. 2019, 30, 2958-2966.
[25] L. Holtzer, I. Oleinich, M. Anzola, E. Lindberg, K. K. Sadhu, M. Gonzalez-Gaitan, N. Winssinger, ACS Cent. Sci. 2016, 2, 394-400.
[26] A. Salic, T. J. Mitchison, Proc. Natl. Acad. Sci. 2008, 105, 2415.
[27] A. B. Neef, N. W. Luedtke, Proc. Natl. Acad. Sci. 2011, 108, 20404.
[28] Y. Wu, G. Guo, J. Zheng, D. Xing, T. Zhang, ACS Sens. 2019, 4, 44-51.
[29] H. Bußkamp, E. Batroff, A. Niederwieser, O. S. Abdel-Rahman, R. F. Winter, V. Wittmann, A. Marx, Chem. Commun. 2014, 50, 10827-10829.
[30] U. Rieder, N. W. Luedtke, Angew. Chem. Int. Ed. 2014, 53, 9168-9172.
[31] N. W. Luedtke, U. Rieder, Methods and Products from the Reaction of Tetrazines with Nucleic Acid Polymers Bearing Ethenyl Aromatic Groups, 2015, WO2015197655.
[32] M. Tera, Z. Harati Taji, N. W. Luedtke, Angew. Chem. Int. Ed. 2018, 57, 15405-15409.
[33] M. Tera, N. W. Luedtke, Bioconjug. Chem. 2019, 30, 2991-2997.
[34] L. S. Lerman, Proc. Natl. Acad. Sci. 1963, 49, 94.
[35] J. C. T. Carlson, L. G. Meimetis, S. A. Hilderbrand, R. Weissleder, Angew. Chem. Int. Ed. 2013, 52, 6917-6920.
[36] A. Wieczorek, P. Werther, J. Euchner, R. Wombacher, Chem. Sci. 2017, 8, 1506-1510.
[37] A. H.-J. Wang, G. J. Quigley, A. Rich, Nucleic Acids Res. 1979, 6, 3879-3890.
[38] Y. Qu, P. Pander, O. Vybornyi, M. Vasylieva, R. Guillot, F. Miomandre, F. B. Dias, P. Skabara, P. Data, G. Clavier, P. Audebert, J. Org. Chem. 2020, 85, 3407-3416.
[39] L. V. Hoff, S. D. Schnell, A. Tomio, A. Linden, K. Gademann, Org. Lett. 2021, 23, 5689-5692.
[40] T.-J. Wang, W.-W. Chen, Y. Li, M.-H. Xu, Org. Biomol. Chem. 2015, 13, 6580-6586.
[41] J. Yang, M. R. Karver, W. Li, S. Sahu, N. K. Devaraj, Angew. Chem. Int. Ed. 2012, 51, 5222-5225.
[42] E. Fredericq, C. Houssier, Biopolymers 1972, 11, 2281-2308.
[43] A. M. Pyle, J. P. Rehmann, R. Meshoyrer, C. V. Kumar, N. J. Turro, J. K. Barton, J. Am. Chem. Soc. 1989, 111, 3051-3058.
[44] P. C. Dedon, Curr. Protoc. Nucleic Acid Chem. 2000, 00, 8.1.1-8.1.13.
[45] L. S. Lerman, J. Mol. Biol. 1961, 3, 18-IN14.
[46] J. Kapuscinski, Z. Darzynkiewicz, J. Biomol. Struct. Dyn. 1987, 5, 127-143.
[47] M. Aslanoglu, Anal. Sci. 2006, 22, 439-443.
[48] S. Nafisi, A. A. Saboury, N. Keramat, J.-F. Neault, H.-A. Tajmir-Riahi, J. Mol. Struct. 2007, 827, 35-43.
[49] B. L. Oliveira, Z. Guo, G. J. L. Bernardes, Chem. Soc. Rev. 2017, 46, 4895-4950.
[50] Z. R. Grabowski, K. Rotkiewicz, W. Rettig, Chem. Rev. 2003, 103, 3899-4032.
[51] A. Naik, J. Alzeer, T. Triemer, A. Bujalska, N. W. Luedtke, Angew Chem Int Ed 2017, 56, 10850-10853.
[52] M. L. W. J. Smeenk, J. Agramunt, K. M. Bonger, Curr Opin Chem Biol 2021, 60, 79-88.
[53] M. Kubota, S. Nainar, S. M. Parker, W. England, F. Furche, R. C. Spitale, ACS Chem. Biol. 2019, 14, 1698-1707.
[54] A. Gubu, L. Li, Y. Ning, X. Zhang, S. Lee, M. Feng, Q. Li, X. Lei, K. Jo, X. Tang, Chem.-Eur. J. 2018, 24, 5895-5900.
[55] G. Linden, L. Zhang, F. Pieck, U. Linne, D. Kosenkov, R. Tonner, O. Vázquez, Angew. Chem. Int. Ed. 2019, 58, 12868-12873.

### References—Example 2

[1] M. Fischer, J. Georges, Chemical Physics Letters 1996, 260, 115-118.
[2] C. R. Cantor, M. M. Warshaw, H. Shapiro, Biopolymers 1970, 9, 1059-1077.
[3] H. S. Radeke, R. R. Cesati, A. Purohit, T. D. Harris, S. P. Robinson, M. Yu, D. S. Casebier, C. H. Hu, M. Broekema, D. C. Onthank, Compositions, Methods and Systems for the Synthesis and Use of Imaging Agents, 2013, WO/2013/036869.
[4] M. A. Schiffler, S. Antonysamy, S. N. Bhattachar, K. M. Campanale, S. Chandrasekhar, B. Condon, P. V. Desai, M. J. Fisher, C. Groshong, A. Harvey, M. J. Hickey, N. E. Hughes, S. A. Jones, E. J. Kim, S. L. Kuklish, J. G. Luz, B. H. Norman, R. E. Rathmell, J. R. Rizzo, T. W. Seng, S. J. Thibodeaux, T. A. Woods, J. S. York, X.-P. Yu, J. Med. Chem. 2016, 59, 194-205.
[5] I. López-Duarte, M. V. Martínez-Díaz, E. Schwartz, M. Koepf, P. H. J. Kouwer, A. E. Rowan, R. J. M. Nolte, T. Torres, ChemPlusChem 2012, 77, 700-706.
[6] E. Dubost, C. Fossey, T. Cailly, S. Rault, F. Fabis, J. Org. Chem. 2011, 76, 6414-6420.
[7] J. V. Jun, E. J. Petersson, D. M. Chenoweth, J. Am. Chem. Soc. 2018, 140, 9486-9493.
[8] U. Rieder, N. W. Luedtke, Angew. Chem. Int. Ed. 2014, 53, 9168-9172.
[9] A. Naik, J. Alzeer, T. Triemer, A. Bujalska, N. W. Luedtke, Angew. Chem. Int. Ed. 2017, 56, 10850-10853.
[10] A. H.-J. Wang, G. J. Quigley, A. Rich, Nucleic Acids Res. 1979, 6, 3879-3890.

### References—Example 3

[1] M. Fischer, J. Georges, Chemical Physics Letters 1996, 260, 115-118.
[2] C. R. Cantor, M. M. Warshaw, H. Shapiro, Biopolymers 1970, 9, 1059-1077.
[3] H. S. Radeke, R. R. Cesati, A. Purohit, T. D. Harris, S. P. Robinson, M. Yu, D. S. Casebier, C. H. Hu, M. Broekema, D. C. Onthank, Compositions, Methods and Systems for the Synthesis and Use of Imaging Agents, 2013, WO/2013/036869.
[4] M. A. Schiffler, S. Antonysamy, S. N. Bhattachar, K. M. Campanale, S. Chandrasekhar, B. Condon, P. V. Desai, M. J. Fisher, C. Groshong, A. Harvey, M. J. Hickey, N. E. Hughes, S. A. Jones, E. J. Kim, S. L. Kuklish, J. G. Luz, B. H. Norman, R. E. Rathmell, J. R. Rizzo, T. W. Seng, S. J. Thibodeaux, T. A. Woods, J. S. York, X.-P. Yu, J. Med. Chem. 2016, 59, 194-205.
[5] I. López-Duarte, M. V. Martínez-Díaz, E. Schwartz, M. Koepf, P. H. J. Kouwer, A. E. Rowan, R. J. M. Nolte, T. Torres, ChemPlusChem 2012, 77, 700-706.
[6] E. Dubost, C. Fossey, T. Cailly, S. Rault, F. Fabis, J. Org. Chem. 2011, 76, 6414-6420.
[7] J. V. Jun, E. J. Petersson, D. M. Chenoweth, J. Am. Chem. Soc. 2018, 140, 9486-9493.
[8] U. Rieder, N. W. Luedtke, Angew. Chem. Int. Ed. 2014, 53, 9168-9172.
[9] A. Naik, J. Alzeer, T. Triemer, A. Bujalska, N. W. Luedtke, Angew. Chem. Int. Ed. 2017, 56, 10850-10853.
[10] A. H.-J. Wang, G. J. Quigley, A. Rich, Nucleic Acids Res. 1979, 6, 3879-3890.

### References—Example 4

(1) Ralhan, R.; Kaur, J. Alkylating Agents and Cancer Therapy. Expert Opin. Ther. Pat. 2007, 17 (9), 1061-1075. https://doi.org/10.1517/13543776.17.9.1061.
(2) Fu, D.; Calvo, J. A.; Samson, L. D. Balancing Repair and Tolerance of DNA Damage Caused by Alkylating Agents. Nat. Rev. Cancer 2012, 12 (2), 104-120. https://doi.org/10.1038/nrc3185.
(3) Hutchinson, M. A. Development of Bifunctional Alkylating Agents for Association and Migration along DNA. undefined 2016.
(4) Deans, A. J.; West, S. C. DNA Interstrand Crosslink Repair and Cancer. Nat. Rev. Cancer 2011, 11 (7), 467-480. https://doi.org/10.1038/nrc3088.
(5) Guainazzi, A.; Scharer, O. Using Synthetic DNA Interstrand Crosslinks to Elucidate Repair Pathways and Identify New Therapeutic Targets for Cancer Chemotherapy. Cell. Mol. Life Sci. 2010. https://doi.org/10.1007/s00018-010-0492-6.
(6) Tera, M.; Harati Taji, Z.; Luedtke, N. W. Intercalation-Enhanced “Click” Crosslinking of DNA. Angew. Chem. Int. Ed. 2018, 57 (47), 15405-15409. https://doi.org/10.1002/anie.201808054.
(7) Melnikov, S. V.; Soll, D.; Steitz, T. A.; Polikanov, Y. S. Insights into RNA Binding by the Anticancer Drug Cisplatin from the Crystal Structure of Cisplatin-Modified Ribosome. Nucleic Acids Res. 2016, 44 (10), 4978-4987. https://doi.org/10.1093/nar/gkw246.
(8) Johnstone, T. C.; Suntharalingam, K.; Lippard, S. J. The Next Generation of Platinum Drugs: Targeted Pt (II) Agents, Nanoparticle Delivery, and Pt (IV) Prodrugs. Chem. Rev. 2016, 116 (5), 3436-3486. https://doi.org/10.1021/acs.chemrev.5b00597.
(9) Branda, R. F.; Hacker, M.; Lafayette, A.; Nigels, E.; Sullivan, L.; Nicklas, J. A.; O'Neill, J. P. Nutritional Folate Deficiency Augments the in Vivo Mutagenic and Lymphocytotoxic Activities of Alkylating Agents. Environ. Mol. Mutagen. 1998, 32 (1), 33-38. https://doi.org/10.1002/(SICI) 1098-2280 (1998) 32: 1<33::AID-EM4>3.0.CO;2-C.
(10) Sletten, E. M.; Bertozzi, C. R. Bioorthogonal Chemistry: Fishing for Selectivity in a Sea of Functionality. Angew. Chem. Int. Ed. 2009, 48 (38), 6974-6998. https://doi.org/10.1002/anie.200900942.
(11) Ganz, D.; Harijan, D.; Wagenknecht, H.-A. Labelling of DNA and RNA in the Cellular Environment by Means of Bioorthogonal Cycloaddition Chemistry. RSC Chem. Biol. 2020, 1 (3), 86-97. https://doi.org/10.1039/D0CB00047G.
(12) Krell, K.; Harijan, D.; Ganz, D.; Doll, L.; Wagenknecht, H.-A. Postsynthetic Modifications of DNA and RNA by Means of Copper-Free Cycloadditions as Bioorthogonal Reactions. Bioconjug. Chem. 2020, 31 (4), 990-1011. https://doi.org/10.1021/acs.bioconjchem.0c00072.
(13) De Kouchkovsky, I.; Abdul-Hay, M. ‘Acute Myeloid Leukemia: A Comprehensive Review and 2016 Update.’ Blood Cancer J. 2016, 6 (7), e441-e441. https://doi.org/10.1038/bcj.2016.50.
(14) Leukemia: Symptoms, Types, Causes & Treatments https://my.clevelandclinic.org/health/diseases/4365-leukemia (accessed 2021 Aug. 31).
(15) Yamamoto, J. F.; Goodman, M. T. Patterns of Leukemia Incidence in the United States by Subtype and Demographic Characteristics, 1997-2002. Cancer Causes Control 2008, 19 (4), 379-390. https://doi.org/10.1007/s10552-007-9097-2.
(16) Brunning, R. D. Classification of Acute Leukemias. Semin. Diagn. Pathol. 2003, 20 (3), (16) 142-153. https://doi.org/10.1016/s0740-2570 (03) 00031-5.
(17) Acute myeloid leukemia: 2014 Update on risk-stratification and management-Estey-2014-American Journal of Hematology-Library Wiley Online https://onlinelibrary.wiley.com/doi/full/10.1002/ajh.23834 (accessed 2021 Aug. 31).
(18) Rieder, U.; Luedtke, N. W. Alkene-Tetrazine Ligation for Imaging Cellular DNA. Angew. Chem. Int. Ed. 2014, 53 (35), 9168-9172. https://doi.org/10.1002/anie.201403580.
(19) Loehr, M. O.; Luedtke, N. W. A Dual Enhancement Strategy for Labeling of Vinyl Nucleosides. 9.
(20) Quentmeier, H.; Reinhardt, J.; Zaborski, M.; Drexler, H. G. FLT3 Mutations in Acute Myeloid Leukemia Cell Lines. Leukemia 2003, 17 (1), 120-124. https://doi.org/10.1038/sj.leu.2402740.
(21) Daver, N.; Schlenk, R. F.; Russell, N. H.; Levis, M. J. Targeting FLT3 Mutations in AML: Review of Current Knowledge and Evidence. Leukemia 2019, 33 (2), 299-312. https://doi.org/10.1038/s41375-018-0357-9.
(22) Matsuo, Y.; MacLeod, R. A.; Uphoff, C. C.; Drexler, H. G.; Nishizaki, C.; Katayama, Y.; Kimura, G.; Fujii, N.; Omoto, E.; Harada, M.; Orita, K. Two Acute Monocytic Leukemia (AML-M5a) Cell Lines (MOLM-13 and MOLM-14) with Interclonal Phenotypic Heterogeneity Showing MLL-AF9 Fusion Resulting from an Occult Chromosome Insertion, Ins (11;9) (Q23;P22p23). Leukemia 1997, 11 (9), 1469-1477. https://doi.org/10.1038/sj.leu.2400768.
(23) AMOL (Acute Monoblastic/Monocytic Leukemia)|(M5) Flow Cytometry https://wiki.clinicalflow.com/amol-acute-monoblasticmonocytic-leukemia-m5 (accessed 2021 Aug. 31).
(24) Labs, A. Altogen Labs Offers Xenograft Services for Drug Development of Anti-Cancer Therapeutics|Altogen Labs.
(25) Sak, K.; Everaus, H. Established Human Cell Lines as Models to Study Anti-Leukemic Effects of Flavonoids. Curr. Genomics 2017, 18 (1), 3-26. https://doi.org/10.2174/1389202917666160803165447.
(26) Resazurin Cell Viability Assay 1 Creative Bioarray https://www.creative-bioarray.com/support/resazurin-cell-viability-assay.htm (accessed 2021 Aug. 31). (27) Eoin, P. M. Investigations into the Responses of Acute Myeloid Leukaemia to Modified Nucleosides. 144.
(28) NanoEntek http://www.nanoentek.com/theme/nanont2_en/shop/02/product01_view.php?it_id=1557725383 (accessed 2021 Aug. 31).
(29) Frances, A.; Cordelier, P. The Emerging Role of Cytidine Deaminase in Human Diseases: A New Mol. Opportunity for Therapy? Ther. 2020, 28 (2), 357-366. https://doi.org/10.1016/j.ymthe.2019.11.026.
(30) Chou, T.-C. Drug Combination Studies and Their Synergy Quantification Using the Chou-Talalay Method. Cancer Res. 2010, 70 (2), 440-446. https://doi.org/10.1158/0008-5472.CAN-09-1947.
(31) Jia, J.; Zhu, F.; Ma, X.; Cao, Z. W.; Li, Y. X.; Chen, Y. Z. Mechanisms of Drug Combinations: Interaction and Network Perspectives. Nat. Rev. Drug Discov. 2009, 8 (2), 111-128. https://doi.org/10.1038/nrd2683.
(32) Humeniuk, R.; Menon, L. G.; Mishra, P. J.; Saydam, G.; Longo-Sorbello, G. S. A.; Elisseyeff, Y.; Lewis, L. D.; Aracil, M.; Jimeno, J.; Bertino, J. R.; Banerjee, D. Aplidin Synergizes with Cytosine Arabinoside: Functional Relevance of Mitochondria in Aplidin-Induced Cytotoxicity. Leukemia 2007, 21 (12), 2399-2405. https://doi.org/10.1038/sj.leu.2404911.
(33) Chou, T. C.; Martin, N. CompuSyn for Drug Combinations: PC Software and User's Guide: A Computer Program for Quantitation of Synergism and Antagonism in Drug Combinations, and the Determination of IC50 and ED50 and LD50 Values. ComboSyn Paramus NJ 2005.

1. A compound having a formula: 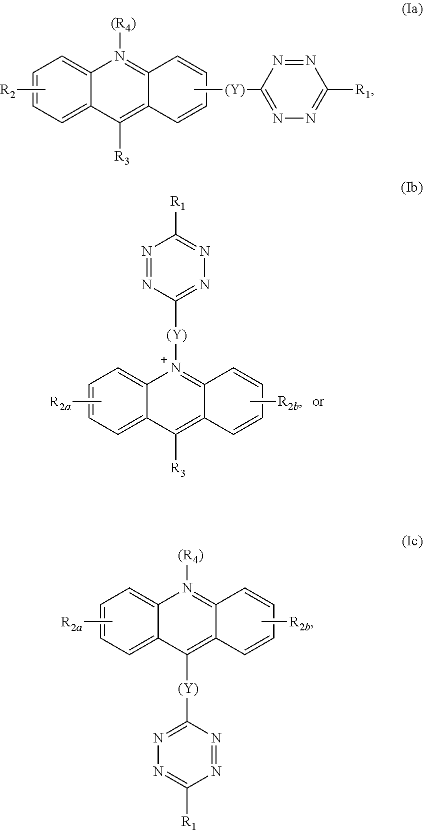
or a salt thereof;
wherein:
R₁ is H, haloalkyl, alkyl, alkenyl, alkynyl, cycloalkyl, aryl, heteroaryl, heterocyclyl, or an optionally substituted group thereof;
R₂, R₂ₐ, R_{2b}, and R₃ are independently H, hydroxy, halogen, haloalkyl, cyano, amino, thiol, alkyl, alkenyl, alkynyl, cycloalkyl, aryl, heteroaryl, heterocyclyl, alkoxy, sulfenyl, acyl, sulfinyl, sulfonyl, O-carboxy, ester, thiocarbonyl, or an optionally substituted group thereof;
R₄ is an optional group and is H, haloalkyl, alkyl, alkenyl, alkynyl, cycloalkyl, aryl, heteroaryl, heterocyclyl, or an optionally substituted group thereof; and
Y is an optional linker;
provided that the compound is not

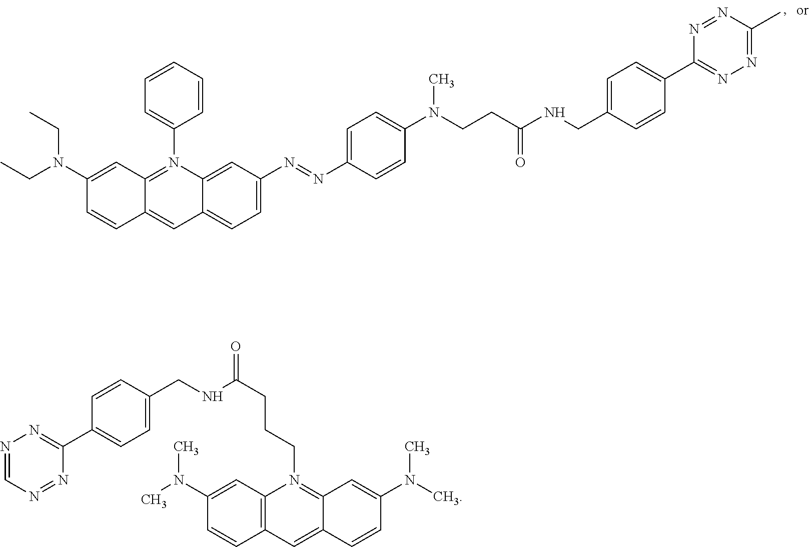

2. The compound or salt of claim 1, wherein R₁ is H, C₁₋₆ alkyl, or optionally substituted aryl or heteroaryl.

3. The compound or salt of claim 2, wherein R₁ is C₁₋₃ alkyl, optionally substituted pyridyl, optionally substituted pyrimidinyl, or optionally substituted C₆₋₁₀ aryl.

4. The compound or salt of claim 3, wherein R₁ is CH₃, pyridyl, pyrimidinyl, or phenyl.

5. The compound or salt of claim 4, wherein R₁ is CH₃.

6. The compound or salt of claim 1, wherein R₂, R₂ₐ, and R_{2b}, are independently H, alkyl, amino, C₆₋₁₀ cycloalkyl, C₆₋₁₀ aryl, heteroaryl, heterocyclyl, or an optionally substituted group thereof.

7. The compound or salt of claim 6, wherein R₂, R₂ₐ, and R_{2b}, are independently H, C₁₋₆ alkyl, optionally substituted C₁₋₆ alkyl, amino, optionally substituted amino, optionally substituted heterocyclyl, or optionally substituted heterocyclyl that forms a ring with the acridine moiety.

8. The compound or salt of claim 7, wherein the compound has formula (Ia), and R₂ is amino substituted with C₁₋₆ alkyl, or optionally substituted heterocyclyl that forms a ring with the acridine moiety.

9. The compound or salt of claim 8, wherein R₂ is dimethylamine.

10. The compound or salt of claim 8, wherein R₂ forms a julolidine group with the acridine moiety.

11. The compound or salt of claim 7, wherein the compound has formula (Ib) or (Ic), and R₂ₐ and R_{2b} are independently H, amino substituted with C₁₋₆ alkyl, or optionally substituted heterocyclyl that forms a ring with the acridine moiety.

12. The compound or salt of claim 11, wherein one of or both of R₂ₐ and R_{2b} are dimethylamine.

13. The compound or salt of claim 11, wherein one of or both of R₂ₐ and R_{2b} forms a ring with the acridine moiety.

14. The compound or salt of claim 13, wherein one of or both of R₂ₐ and R_{2b} forms a julolidine group with the acridine moiety.

15. The compound or salt of claim 1, wherein R₃ is H, alkyl, heteroalkyl, aryl, heteroaryl, or an optionally substituted group thereof.

16. The compound or salt of claim 15, wherein R₃ is H, optionally substituted C₁₋₆ alkyl, C₁₋₆ heteroalkyl, optionally substituted 5 or 6 membered heteroaryl, or optionally substituted C₆₋₁₀ aryl.

17. The compound or salt of claim 1, wherein R₄ is absent.

18. The compound or salt of claim 1, wherein R₄ is H or C₁₋₆ alkyl.

19. The compound or salt of claim 18, wherein R₄ is H or CH₃.

20. The compound or salt of claim 1, wherein Y is alkyl, heteroalkyl, aryl, heteroaryl, alkenyl, alkynyl, alkylthio, or an optionally substituted group thereof.

21. The compound or salt of claim 20, wherein Y is optionally substituted C₁₋₆ alkyl, optionally substituted C₁₋₆ heteroalkyl, optionally substituted C₆₋₁₀ aryl, optionally substituted 5 or 6 membered heteroaryl, optionally substituted C₁₋₆ alkenyl, optionally substituted C₁₋₆ alkynyl, or optionally substituted C₁₋₆ alkylthio.

22. The compound or salt of claim 21, wherein Y is C₁₋₃ alkyl, or C₁₋₃ alkylthio.

23. The compound or salt of claim 1, wherein Y is absent.

24. The compound or salt of claim 1, having formula:

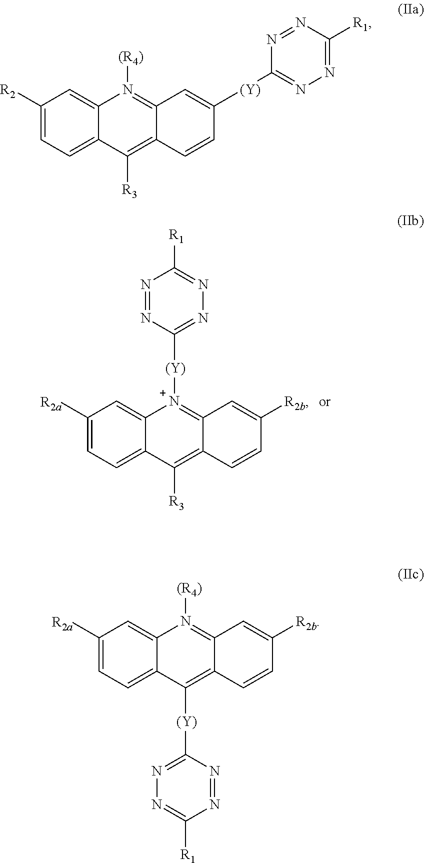

25. The compound or salt of claim 1 having formula (IIIb)

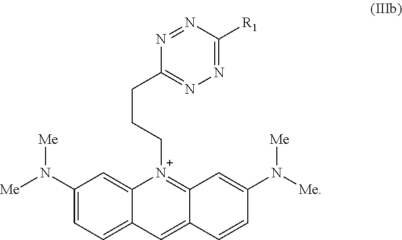

26. The compound or salt of claim 1, wherein the compound is:

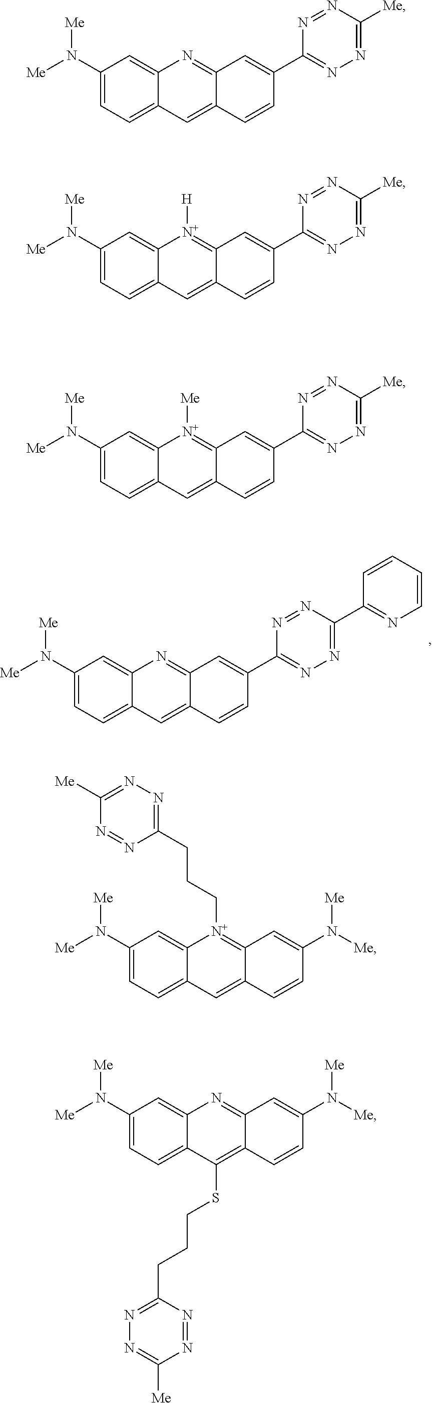 [SMILES: Cc1nnc(-c2ccc3cc4ccc(N(C)C)cc4nc3c2)nn1 ; Cc1nnc(-c2ccc3cc4ccc(N(C)C)cc4[nH+]c3c2)nn1 ; Cc1nnc(-c2ccc3cc4ccc(N(C)C)cc4[n+](C)c3c2)nn1 ; CN(C)c1ccc2cc3ccc(-c4nnc(-c5ccccn5)nn4)cc3nc2c1 ; Cc1nnc(CCC[n+]2c3cc(N(C)C)ccc3cc3ccc(N(C)C)cc32)nn1 ; Cc1nnc(CCCSc2c3ccc(N(C)C)cc3nc3cc(N(C)C)ccc23)nn1]

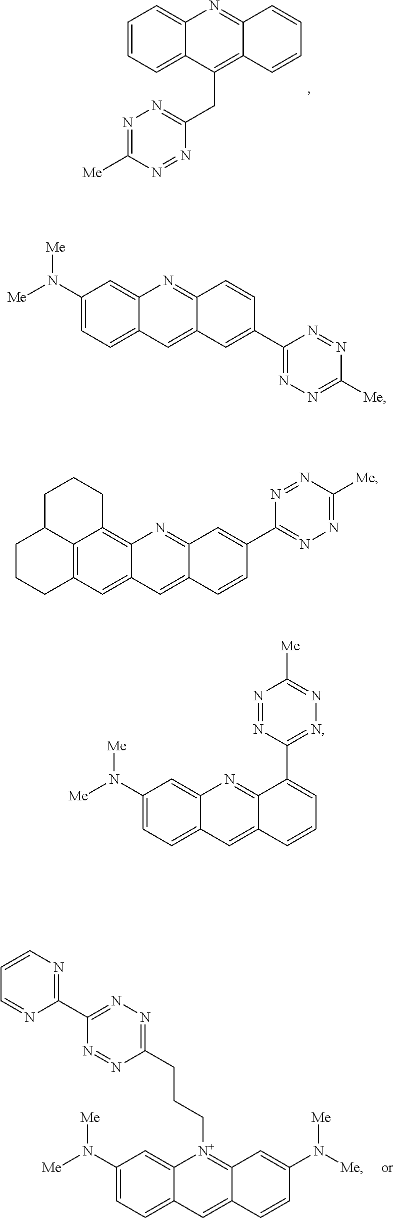 [SMILES: Cc1nnc(Cc2c3ccccc3nc3ccccc23)nn1 ; Cc1nnc(-c2ccc3nc4cc(N(C)C)ccc4cc3c2)nn1 ; Cc1nnc(-c2ccc3cc4cc5c6c(c4nc3c2)CCCC6CCC5)nn1 ; Cc1nnc(-c2cccc3cc4ccc(N(C)C)cc4nc23)nn1 ; CN(C)c1ccc2cc3ccc(N(C)C)cc3[n+](CCCc3nnc(-c4ncccn4)nn3)c2c1]

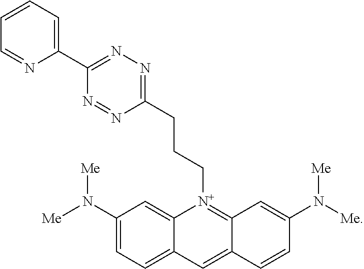 [SMILES: CN(C)c1ccc2cc3ccc(N(C)C)cc3[n+](CCCc3nnc(-c4ccccn4)nn3)c2c1]

27. The compound or salt of claim 1, being a probe.

28. A method of detecting nucleic acids containing a vinyl-nucleoside, the method comprising:
a) contacting the cell or tissue with the compound or salt of claim 1; and
b) exposing the cell or tissue of a) to light.

29. The method of claim 28, wherein the vinyl-nucleoside is:
5-vinyl-2′-deoxyuridine (VdU),
5-vinyl-2′-deoxycytidine (VdC),
7-vinyl-7-deaza-2′-deoxyadenosine (VdA),
7-vinyl-7-deaza-2′-deoxyguanosine (VdG),
5-vinyluridine (VU),
5-vinylcytidine (VC),
7-vinyl-7-deazaadenosine (VA), or
7-vinyl-7-deaza-2′-deoxyguanosine (VG).

30. The method of claim 29, wherein the vinyl-nucleoside is VdU and/or VdA.

31. The method of claim 29, wherein the vinyl-nucleoside is VU.

32. The method of claim 28, performed in vitro or in vivo.

33. (canceled)

34. A kit for detection of nucleic acid in a cell or tissue, the kit comprising:
one or more vinyl-nucleosides; and
the compound or salt of claim 1.

35. The kit of claim 34, wherein the one or more vinyl-nucleosides comprise one or more of:
5-vinyl-2′-deoxyuridine (VdU),
5-vinyl-2′-deoxycytidine (VdC),
7-vinyl-7-deaza-2′-deoxyadenosine (VdA),
7-vinyl-7-deaza-2′-deoxyguanosine (VdG). 5-vinyluridine (VU),
5-vinylcytidine (VC),
7-vinyl-7-deazaadenosine (VA), and
7-vinyl-7-deaza-2′-deoxyguanosine (VG).

36. A method of treating cancer in a subject that has been administered one or more vinyl-nucleosides, the method comprising administering the compound or salt of claim 1 to the subject.

37. The method of claim 36, wherein the one or more vinyl-nucleosides comprise one or more of:
5-vinyl-2′-deoxyuridine (VdU),
5-vinyl-2′-deoxycytidine (VdC),
7-vinyl-7-deaza-2′-deoxyadenosine (VdA),
7-vinyl-7-deaza-2′-deoxyguanosine (VdG),
5-vinyluridine (VU),
5-vinylcytidine (VC),
7-vinyl-7-deazaadenosine (VA), and
7-vinyl-7-deaza-2′-deoxyguanosine (VG).

38. A pharmaceutical composition comprising the compound or salt of claim 1 and a acceptable pharmaceutical carrier.

39. (canceled)

40. (canceled)

41. (canceled)

42. (canceled)
